(19) United States
(12) Patent Application Publication
(10) Pub. No.: US 2023/0381159 A1
(43) Pub. Date: Nov. 30, 2023

(54) SMALL MOLECULE INHIBITORS OF CORONAVIRUS ATTACHMENT AND ENTRY, METHODS AND USES THEREOF

(71) Applicant: UNIVERSITY OF MIAMI, Miami, FL (US)

(72) Inventor: Peter Buchwald, Miami, FL (US)

(21) Appl. No.: 18/029,025

(22) PCT Filed: Sep. 29, 2021
(86) PCT No.: PCT/US21/52520

§ 371 Date: Mar. 28, 2023

Related U.S. Application Data
(60) Provisional application No. 63/085,008, filed on Sep. 29, 2020.

(51) Int. Cl. A61K 31/44; A61K 31/166; A61P 31/14

(52) U.S. Cl. CPC A61K 31/44; A61K 31/166; A61P 31/14

(57) ABSTRACT

Provided herein are small molecule inhibitors of coronavirus attachment and entry, related pharmaceutical compositions, uses, and methods thereof, wherein the compound has a structure of Formula (I):

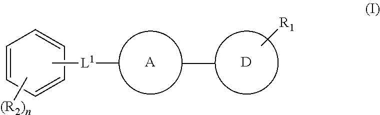

### CROSS-REFERENCE TO RELATED APPLICATIONS

This application claims the benefit under 35 U.S.C. § 119(e) of U.S. Provisional Patent Application No. 63/085,008, filed Sep. 29, 2020, the entire disclosure of which is incorporated herein by reference.

### BACKGROUND

### Field of Disclosure

The present disclosure relates to small molecule inhibitors of the protein-protein interactions (PPIs) between coronavirus spike proteins and their cognate cell receptors (e.g., ACE2 for SARS-CoV-2), which are used for host attachment and initiation of viral entry, and methods of using these small molecules as prevention and treatment by inhibiting viral attachment and entry for SARS-CoV-2 and other coronaviruses.

### Brief Description of Related Technology

Severe acute respiratory syndrome-coronavirus 2 (SARS-CoV-2), a novel betacoronavirus and the most recent one of the seven coronaviruses (CoVs) known to infect humans, is responsible for COVID-19, which was declared a pandemic by the World Health Organization in March 2020. While four CoVs (HCoV-229E, -OC43, -NL63, and -HKU1) have been responsible for about one-third of the common cold cases in humans, three CoVs have caused recent epidemics associated with considerable mortality: SARS-CoV-1 (2002-2003, causing ˜10% mortality), MERS-CoV (Middle East respiratory syndrome coronavirus; 2012, causing ˜35% mortality), and SARS-CoV-2 (2019-2020), which is less lethal but more transmissible.

COVID-19 is the most infectious agent in a century and has caused infections in the order of hundreds of millions and deaths that are in the order of millions worldwide. According to early estimates, about 3% of infected individuals need hospitalization and 0.5% die—a range that is strongly age-dependent, i.e., increasing log-linearly from 0.001% in <20 years old to 8.3% in those >80 years old.

CoVs use their glycosylated S protein to bind to their cognate cell surface receptors and initiate membrane fusion and virus entry. For both SARS-CoV and SARS-CoV-2, the spike protein S mediates entry into cells by binding to angiotensin converting enzyme 2 (ACE2) via its receptor-binding domain (RBD) followed by proteolytic activation by human proteases. Blockade of this RBD-ACE2 protein-protein interaction (PPI) can disrupt infection efficiency and provide antiviral activity.

Antibodies can be effective PPI inhibitors, as they are highly target-specific and relatively stable in vivo. However, they cannot reach intracellular targets and, as all other protein therapies, are hindered by problems such as low solubility, propensity for immunogenicity, long elimination half-lives, lack of oral bioavailability, product heterogeneity, and possible manufacturing and storage stability issues. Moreover, since antibodies are foreign proteins, they elicit strong immune response in certain patients, and even if approved for clinical use, they tend to have more post-market safety issues than small-molecule drugs. Furthermore, current evidence indicates that most SARS-CoV antibodies will not be cross-reactive for SARS-CoV-2. For example, one study found that none of the 206 RBD-specific monoclonal antibodies derived from single B cells of eight SARS-CoV-2 infected individuals cross-reacted with SARS-CoV or MERS-CoV RBDs. Additionally, RNA viruses are known to accumulate mutations over time, and this can yield antibody resistance over time making the use of antibody cocktail a necessity to avoid mutational escape. Not surprisingly, several SARS-CoV-2 mutants representing variants of concern (VOC) have already emerged that show increased transmissibility, higher diseases severity, and/or resistance to neutralizing antibodies.

The success of the COVID-19 vaccination program notwithstanding, there remains a considerable interest in developing new antivirals and especially oral treatments as a significant portion of the population is either unwilling to be vaccinated or unable to do so due to pre-existing medical conditions. Accordingly, small molecule inhibitors for prevention and/or therapeutic treatment of viral infections, such as those caused by coronaviruses, are needed.

### SUMMARY

Provided herein are compounds, or salts thereof, of Formula (I):

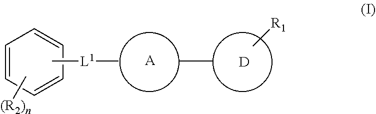

### Wherein Ring A is

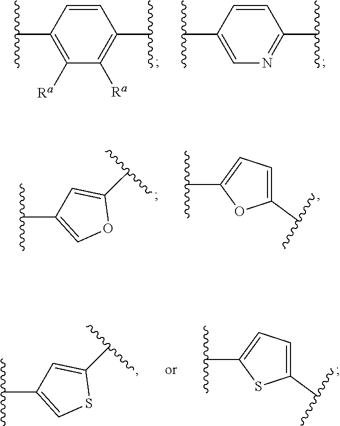

### Ring D is

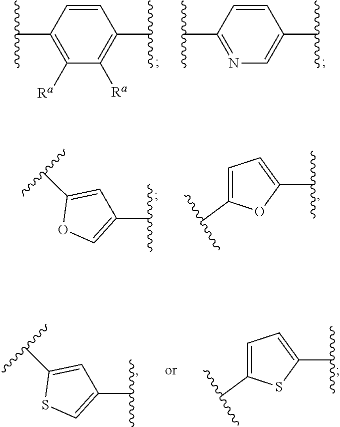

R₁ is H, halo, CF₃, SO₃H, CO₂R^{b}, NO₂, NH₂, or

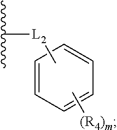

each of L₁ and L₂ independently is

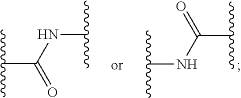 [SMILES: *NC(*)=O ; *NC(*)=O]

n is 0, 1, 2, 3, or 4; m is 0, 1, 2, 3, or 4; each R₂ independently is halo, CF₃, SO₃H, CO₂R^{b}, NO₂, or NH₂; and when two R₂ are adjacent, they can together form —(N═N—NH)— or, with the carbon atoms to which they are attached, form a C₆ aryl optionally substituted with 1-4 R₃; each R₃ independently is halo, OH, CF₃, SO₃H, CO₂R^{b}, NO₂, or NH₂; and when two R₃ are adjacent, they can together form —(N═N—NH)—; each R₄ independently is halo, CF₃, SO₃H, CO₂R^{b}, NO₂, or NH₂; and when two R₄ are adjacent, they can together form —(N═N—NH)— or, with the carbon atoms to which they are attached, form a C₆ aryl optionally substituted with 1-4 R₃; each R^{a} independently is H, C₁₋₅ alkyl, or C₁₋₅ alkoxy; and, each R^{b} independently is H or C₁₋₅ alkyl; or a pharmaceutically acceptable salt thereof;
with the proviso that: (a) if L₁-ring A-ring D-L₂ is

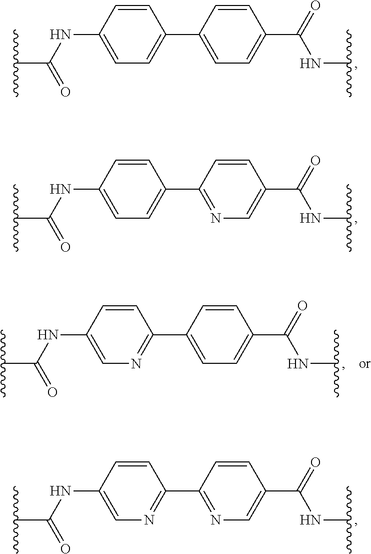 [SMILES: *NC(=O)c1ccc(-c2ccc(NC(*)=O)cc2)cc1 ; *NC(=O)c1ccc(-c2ccc(NC(*)=O)cc2)nc1 ; *NC(=O)c1ccc(-c2ccc(NC(*)=O)cn2)cc1 ; *NC(=O)c1ccc(-c2ccc(NC(*)=O)cn2)nc1]

then two adjacent R₂, with the carbon atoms to which they are attached, form a C₆ aryl, optionally substituted with 1-4 R₃; and (b) if L₁-ring A-ring D-L₂ is

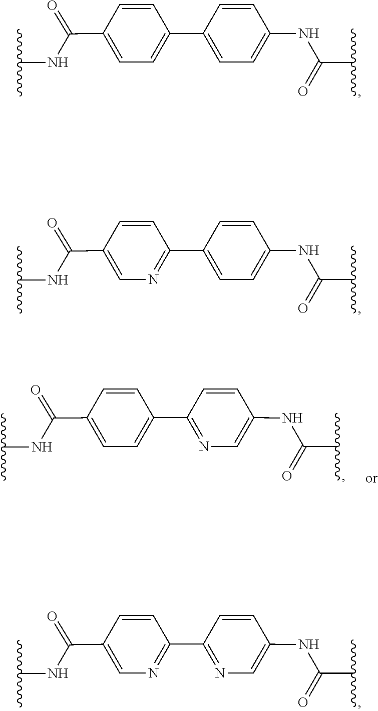 [SMILES: *NC(=O)c1ccc(-c2ccc(NC(*)=O)cc2)cc1 ; *NC(=O)c1ccc(-c2ccc(NC(*)=O)cc2)nc1 ; *NC(=O)c1ccc(-c2ccc(NC(*)=O)cn2)cc1 ; *NC(=O)c1ccc(-c2ccc(NC(*)=O)cn2)nc1]

then two adjacent R₄, with the carbon atoms to which they are attached, form a C₆ aryl, optionally substituted with 1-4 R₃.

In various embodiments, ring A is

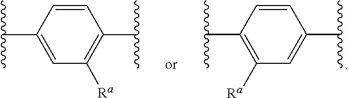

In some cases, R^{a} is H. In some cases, R^{a} is methyl.

In various embodiments, ring A is

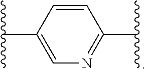 [SMILES: *c1ccc(*)nc1]

In various embodiments, ring A is

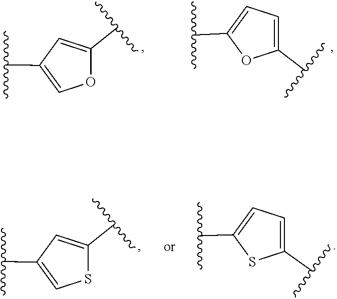 [SMILES: *c1coc(*)c1 ; *c1ccc(*)o1 ; *c1csc(*)c1 ; *c1ccc(*)s1]

In various embodiments, ring D is

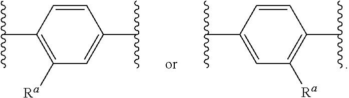

In some cases, R^{a} is H. In some cases, R^{a} is methyl.

In various embodiments, ring D is

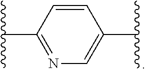 [SMILES: *c1ccc(*)nc1]

In various embodiments, ring D is

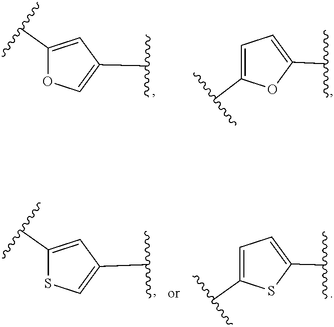 [SMILES: *c1coc(*)c1 ; *c1ccc(*)o1 ; *c1csc(*)c1 ; *c1ccc(*)s1]

In various embodiments,

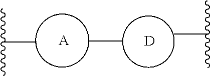

is selected from the group consisting of

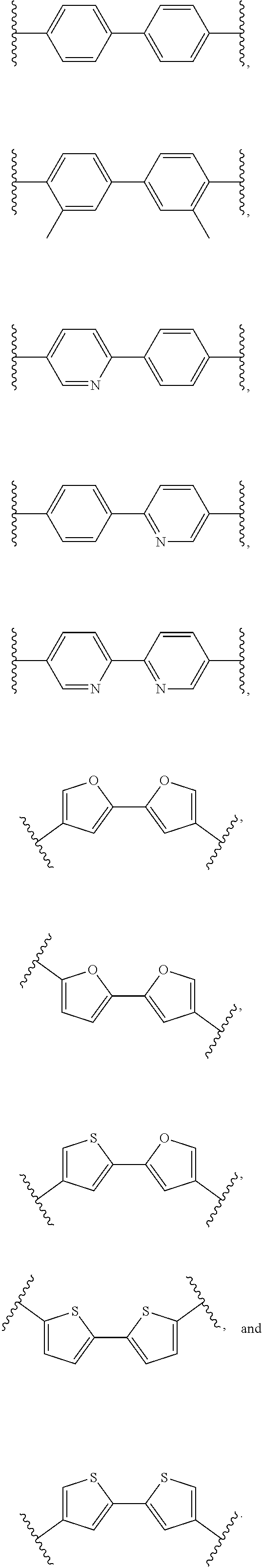

In some cases,

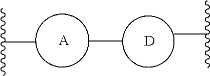

is

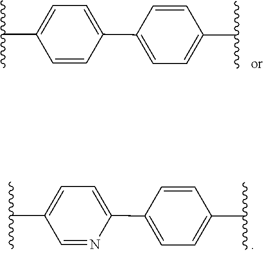 [SMILES: *c1ccc(-c2ccc(*)cc2)cc1 ; *c1ccc(-c2ccc(*)cn2)cc1]

In various embodiments, R₁ is H. In various embodiments, R₁ is halo. In some cases, R₁ is F or Cl. In various embodiments, R₁ is CF₃. In various embodiments, R₁ is SO₃H. In various embodiments, R₁ is CO₂R^{b}. In some cases, R^{b} is H. In some cases, R^{b} is C₁₋₅ alkyl. In various embodiments, R₁ is NO₂. In various embodiments, R₁ is NH₂. In various embodiments, R₁ is

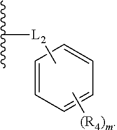

In various embodiments, L₂ is

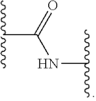 [SMILES: *NC(*)=O]

In various embodiments, L₂ is

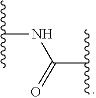 [SMILES: *NC(*)=O]

In various embodiments, m is 0. In various embodiments, m is 1, 2, 3, or 4. In various embodiments, at least one R₄ is halo. In some cases, at least one R₄ is Cl or F. In various embodiments, at least one R₄ is CF₃. In various embodiments, at least one R₄ is SO₃H. In various embodiments, at least one R₄ is CO₂R^{b}. In some cases, R^{b} is H. In some cases, R^{b} is C₁₋₅ alkyl. In various embodiments, at least one R₄ is NO₂. In various embodiments, at least one R₄ is NH₂. In various embodiments, m is at least 2, and two R₄ are adjacent and together with the carbon atoms to which they are attached form a C₆ aryl optionally substituted with 1-4 R₃.

In various embodiments, R₁ is selected from the group consisting of

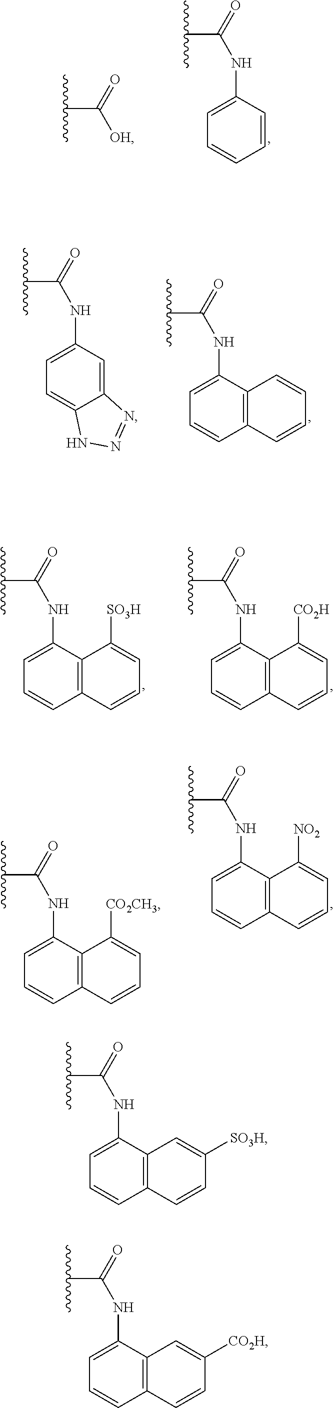

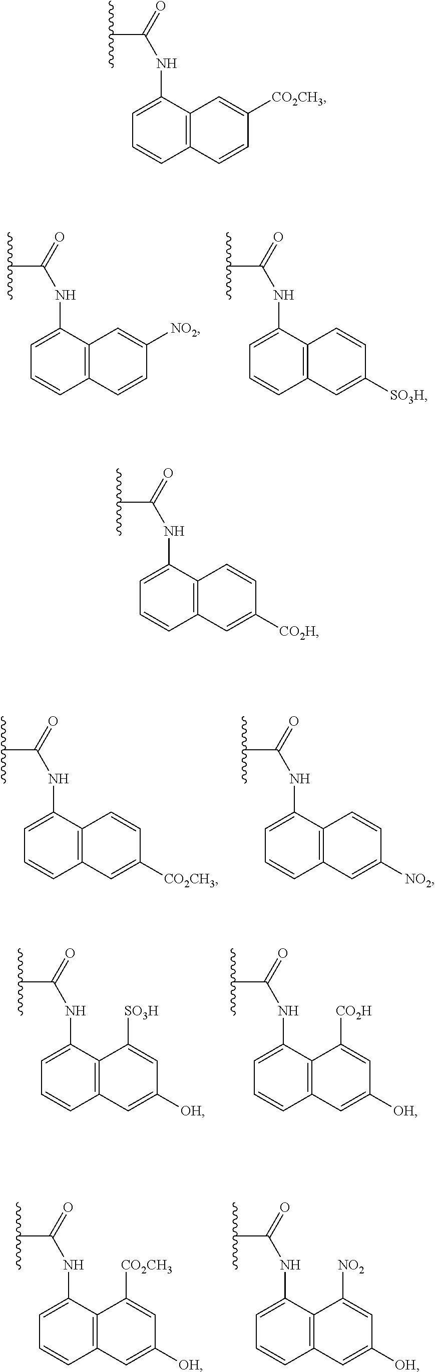

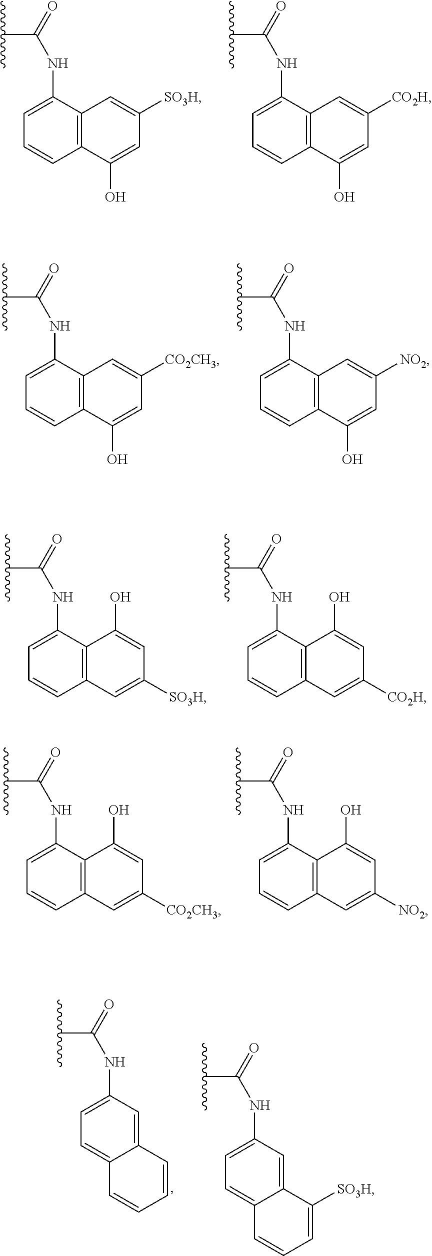

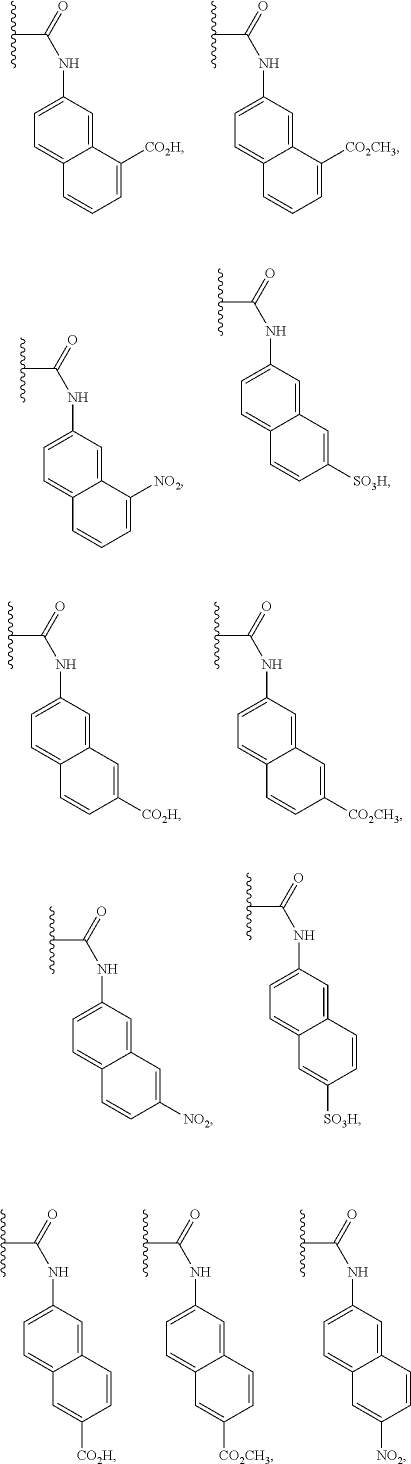

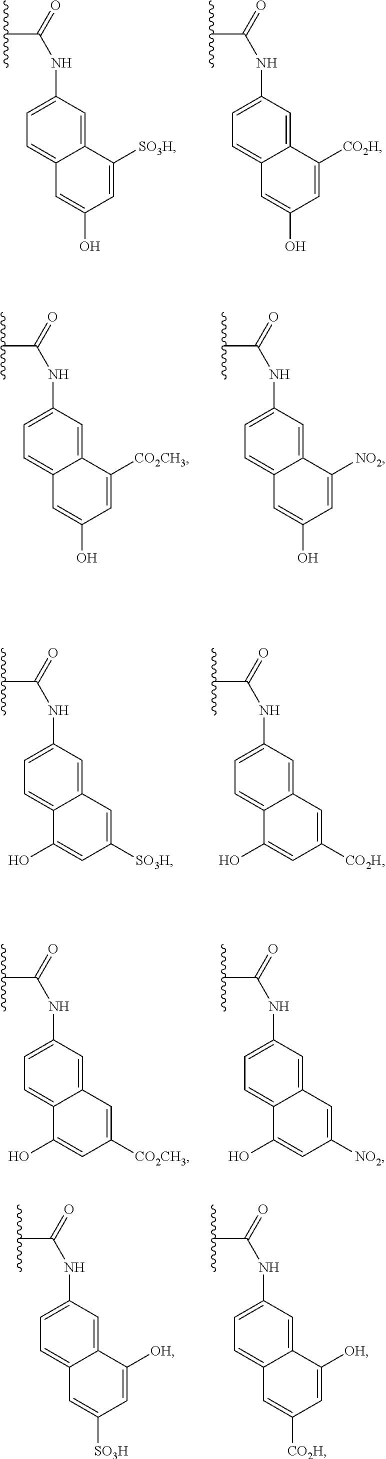

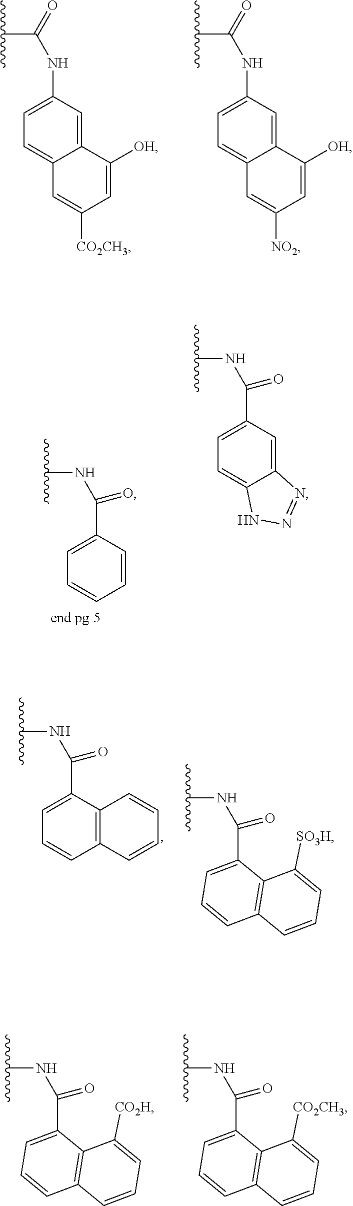

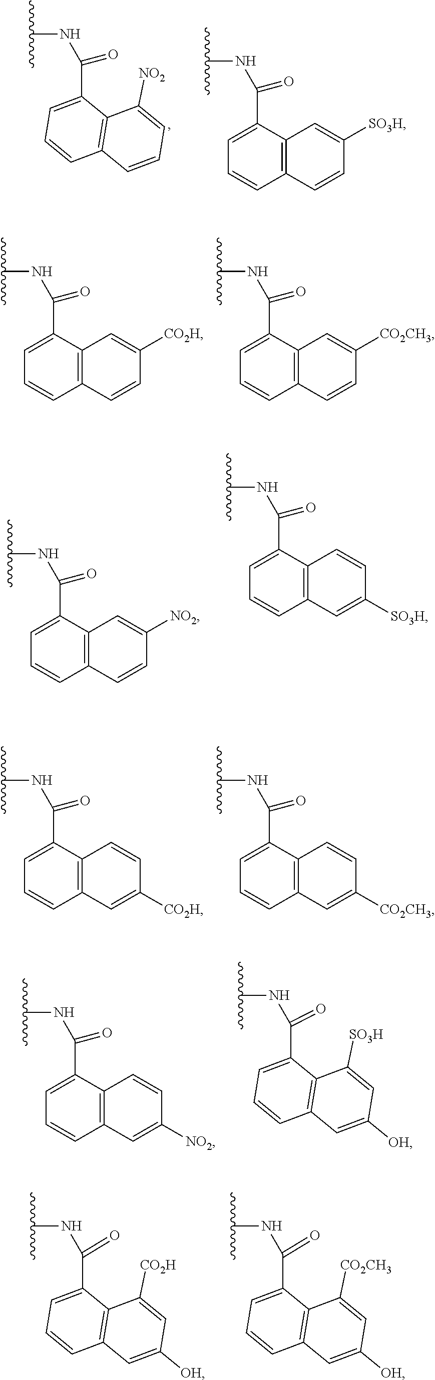

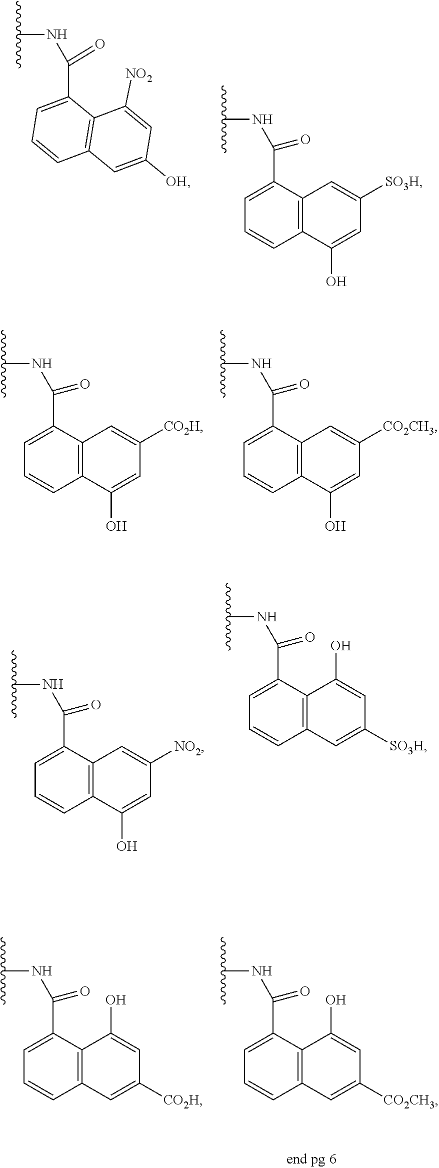

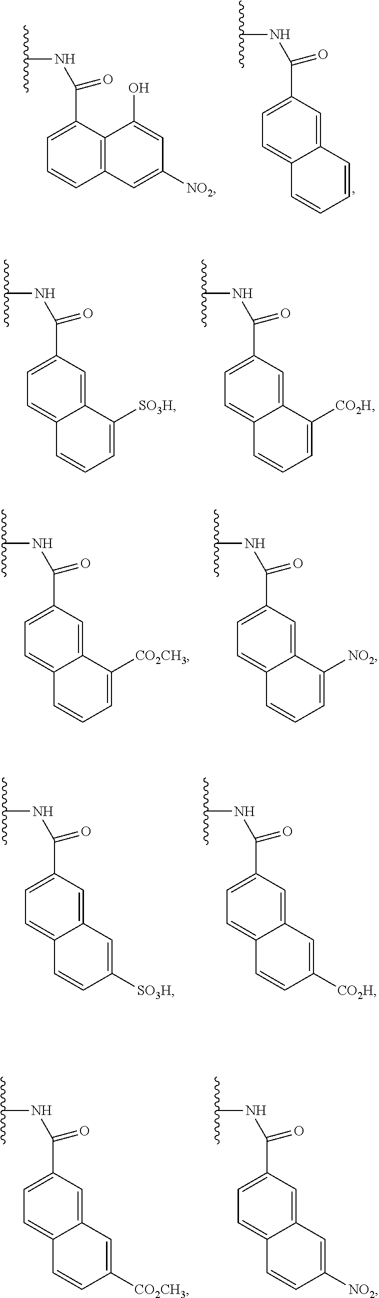

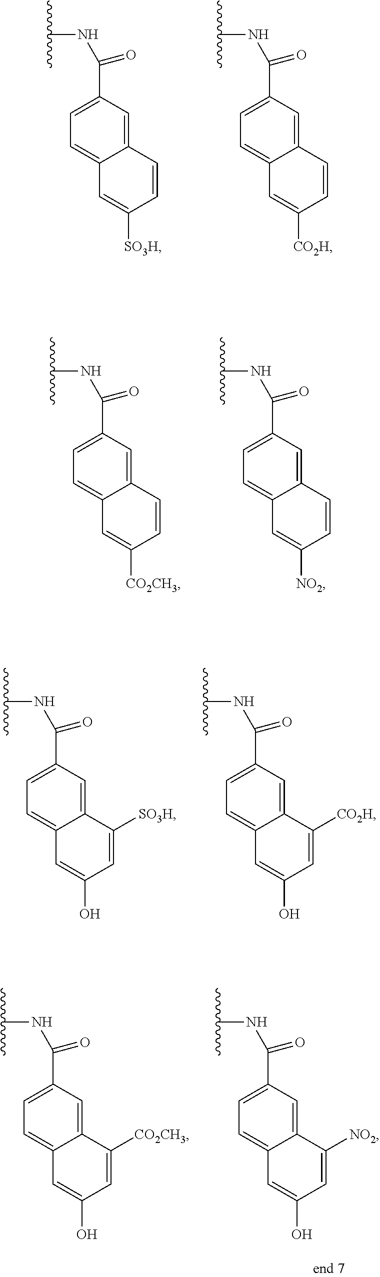

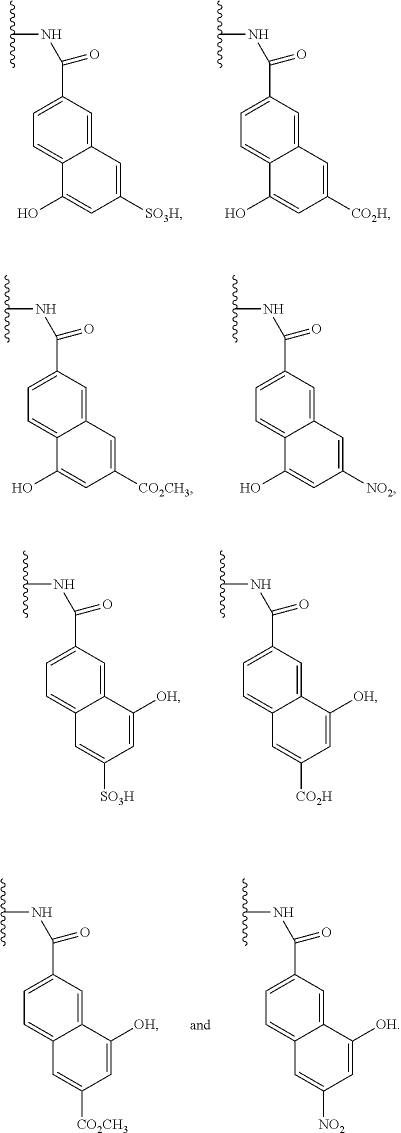

In various embodiments, L₁ is

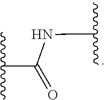 [SMILES: *NC(*)=O]

In various embodiments, L₁ is

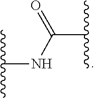 [SMILES: *NC(*)=O]

In various embodiments, n is 0. In various embodiments, n is 1, 2, 3, or 4. In various embodiments, at least one R₂ is halo. In some cases, at least one R₂ is Cl or F. In various embodiments, at least one R₂ is CF₃. In various embodiments, at least one R₂ is SO₃H. In various embodiments, at least one R₂ is CO₂R^{b}. In some cases, R^{b} is H. In some cases, R^{b} is C₁₋₅ alkyl. In various embodiments, at least one R₂ is NO₂. In various embodiments, at least one R₂ is NH₂. In various embodiments, n is at least 2, and two R₂ are adjacent and taken together with the carbon atoms to which they are attached form a C₆ aryl optionally substituted with 1-4 R₃.

In various embodiments,

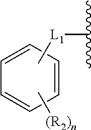

is selected from the group consisting of

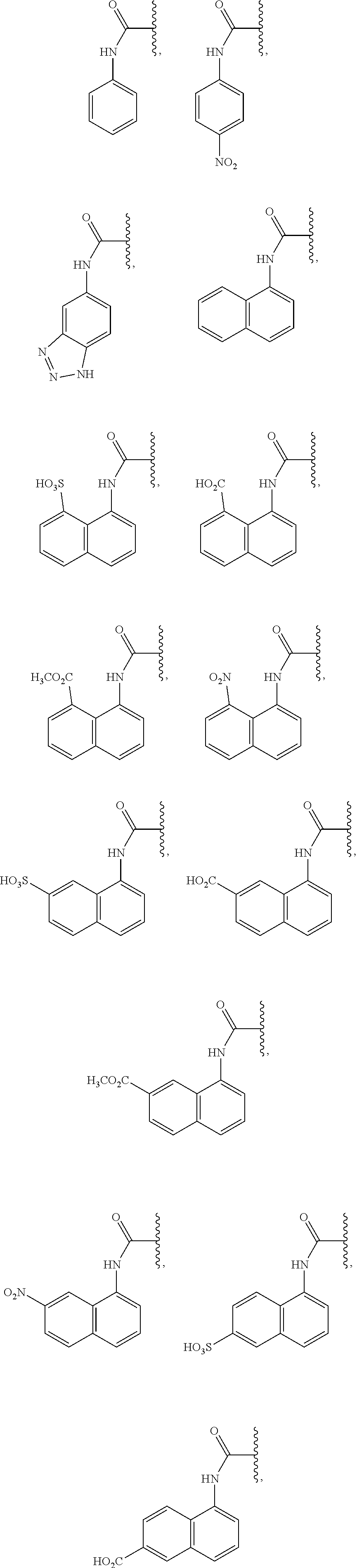

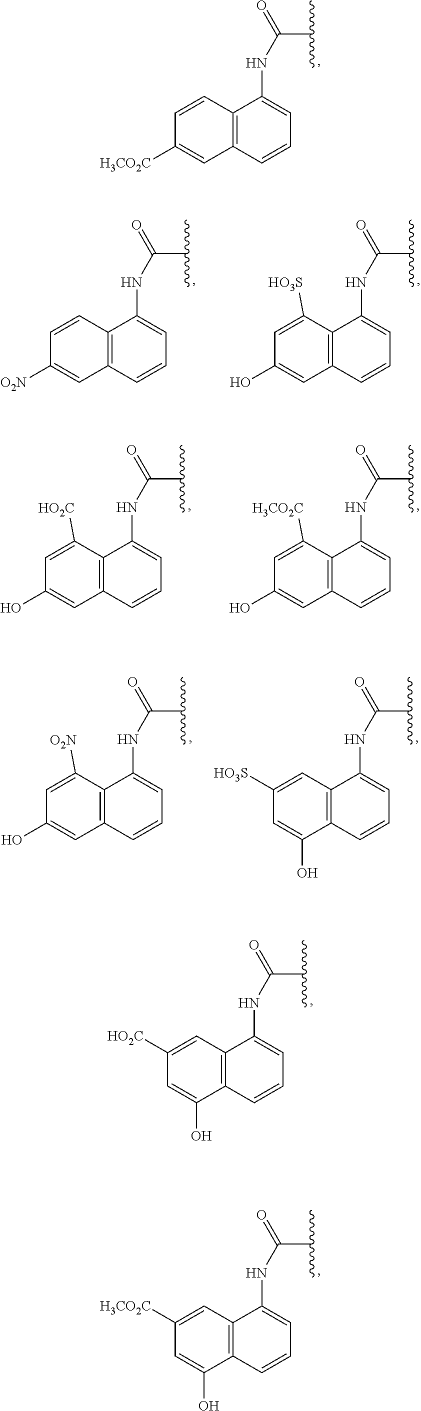

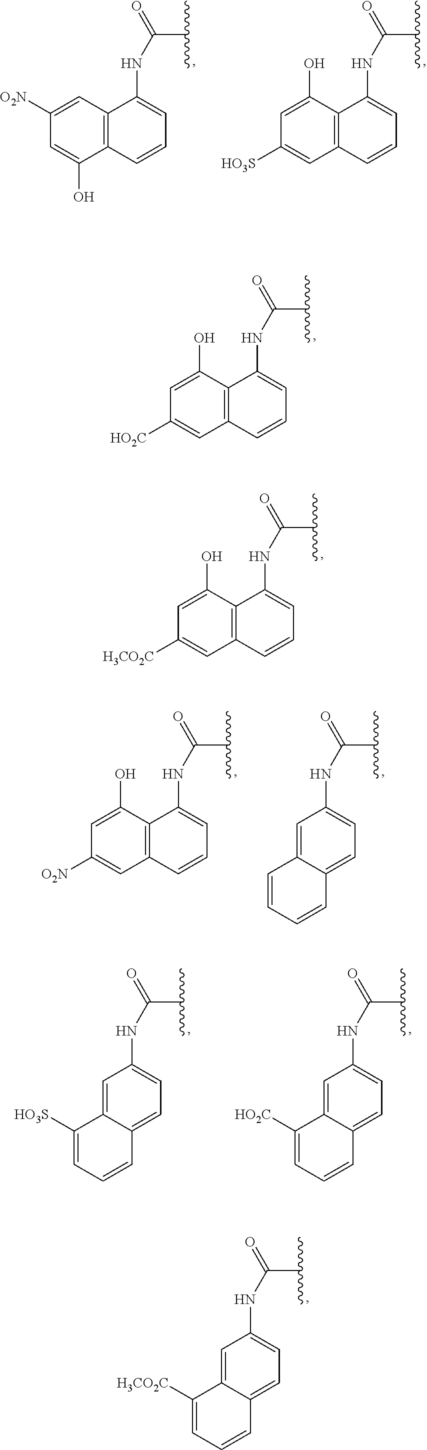

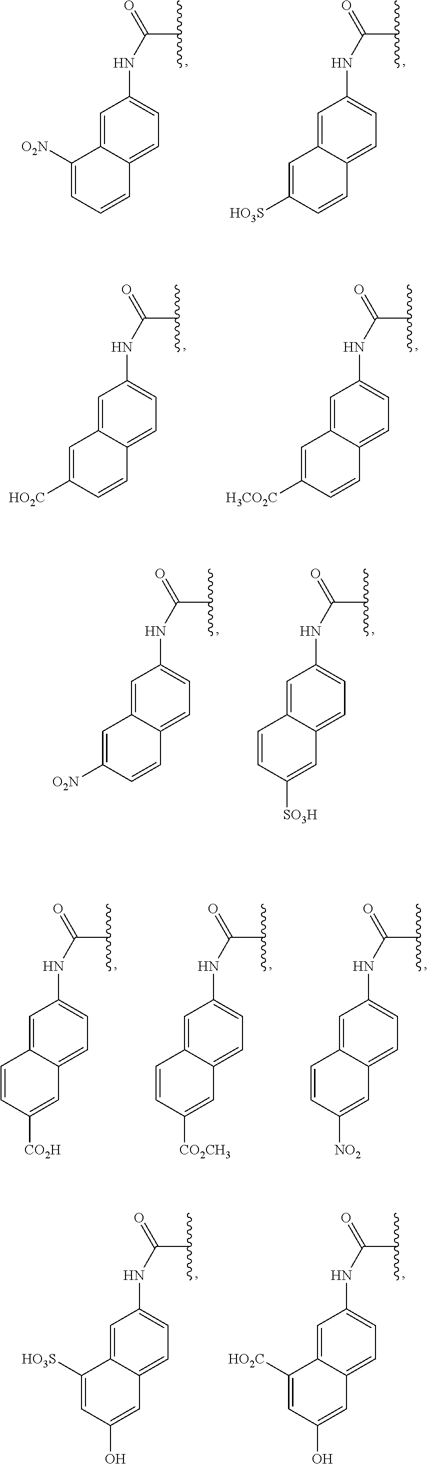

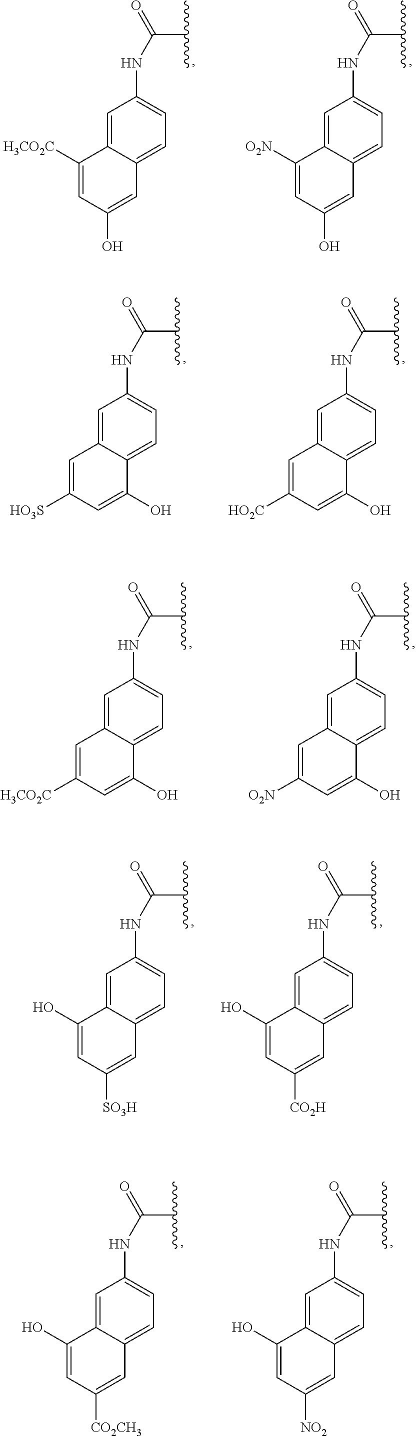

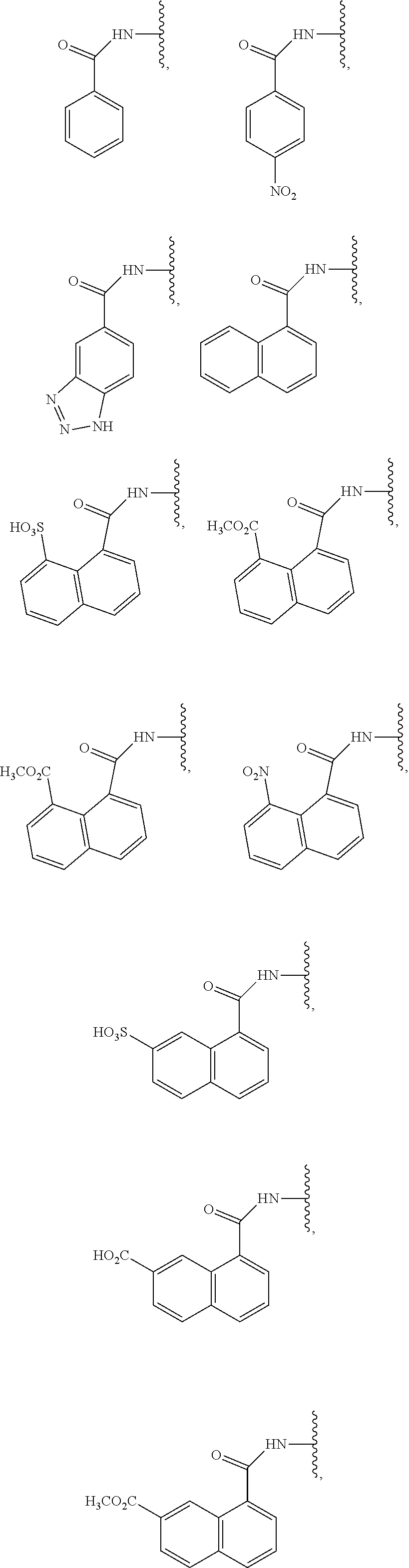

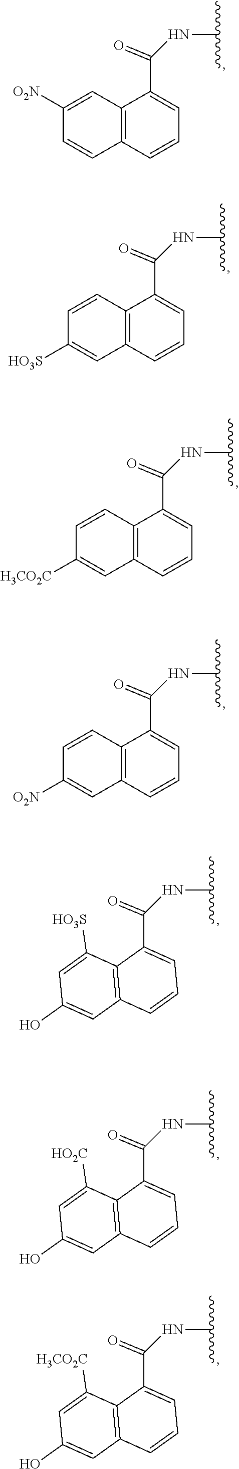

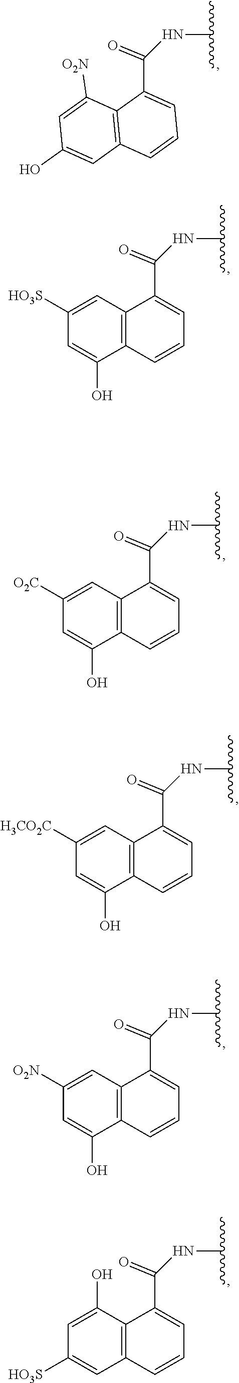

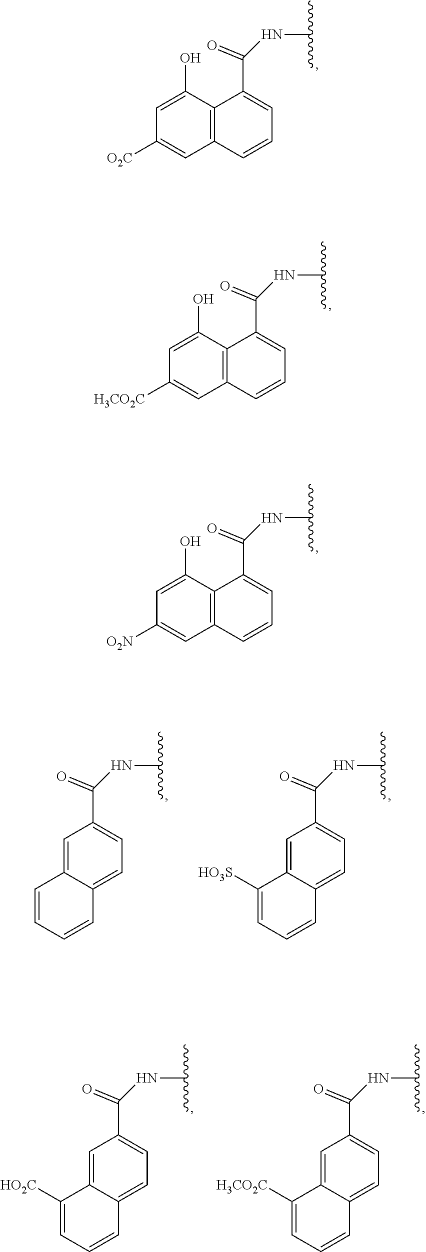

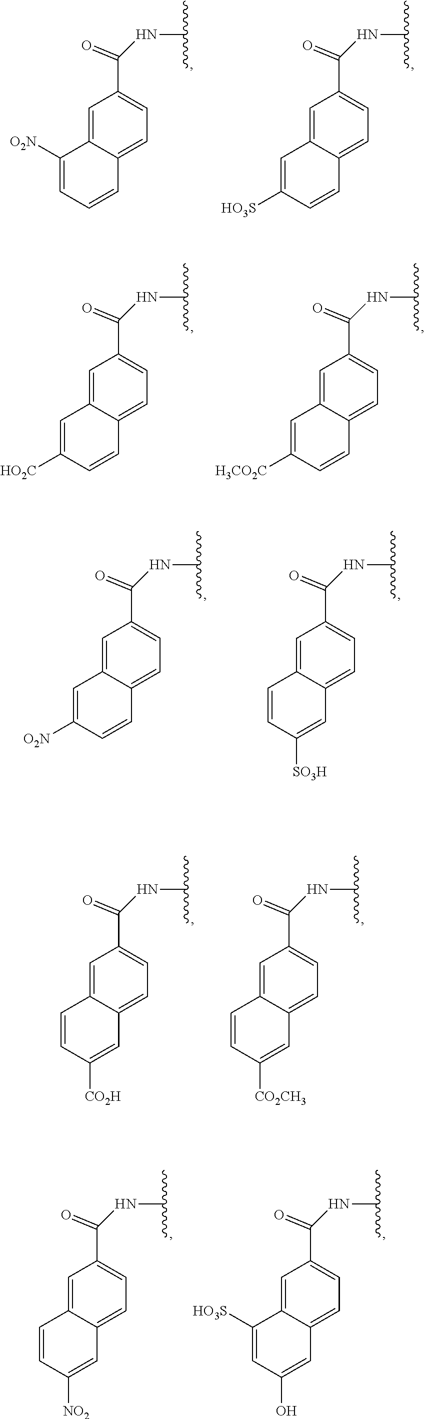

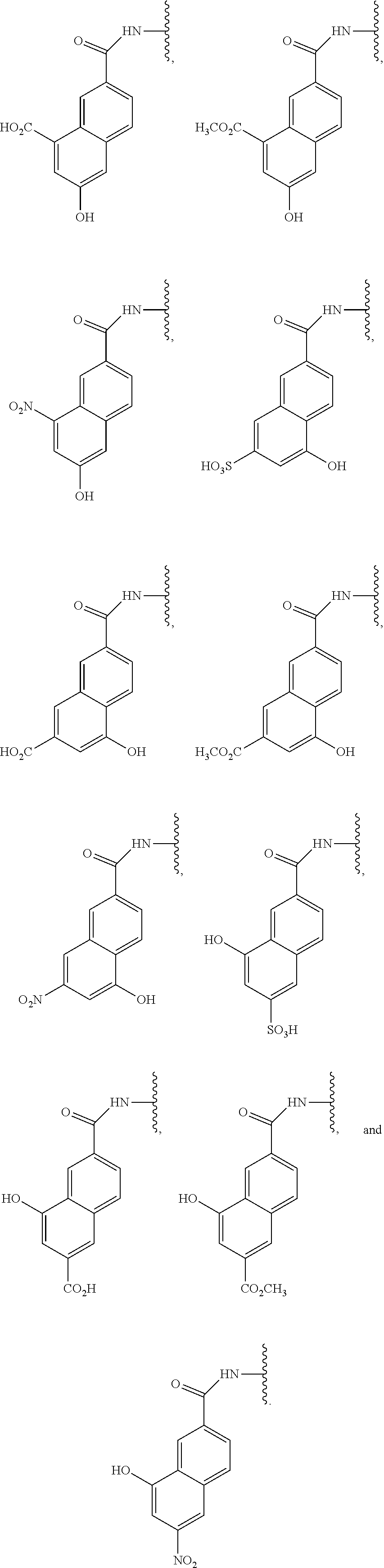

In various embodiments, the compound or salt thereof is selected from the group consisting of

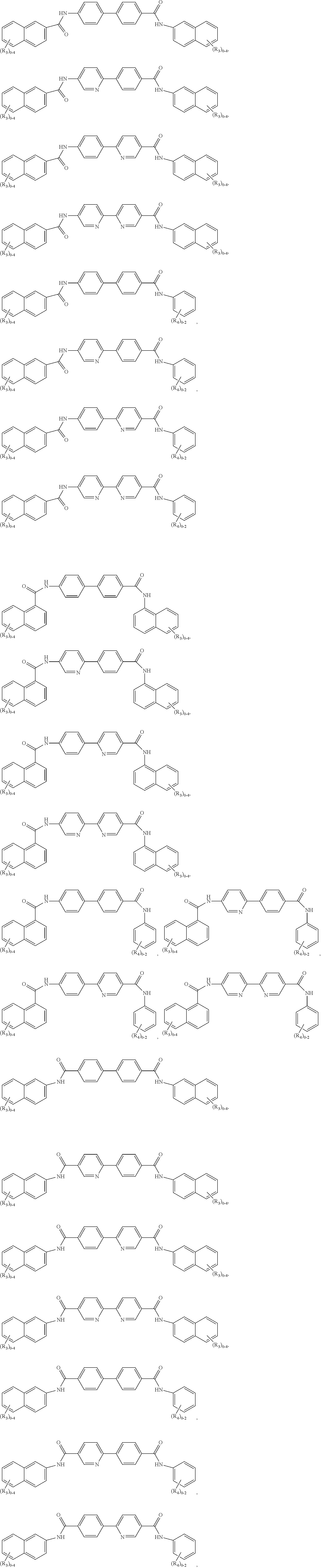

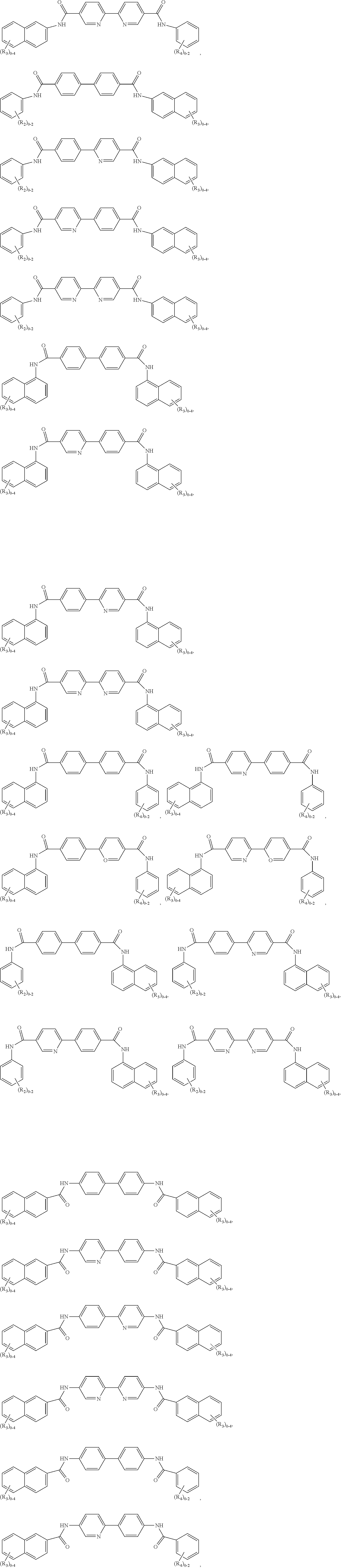

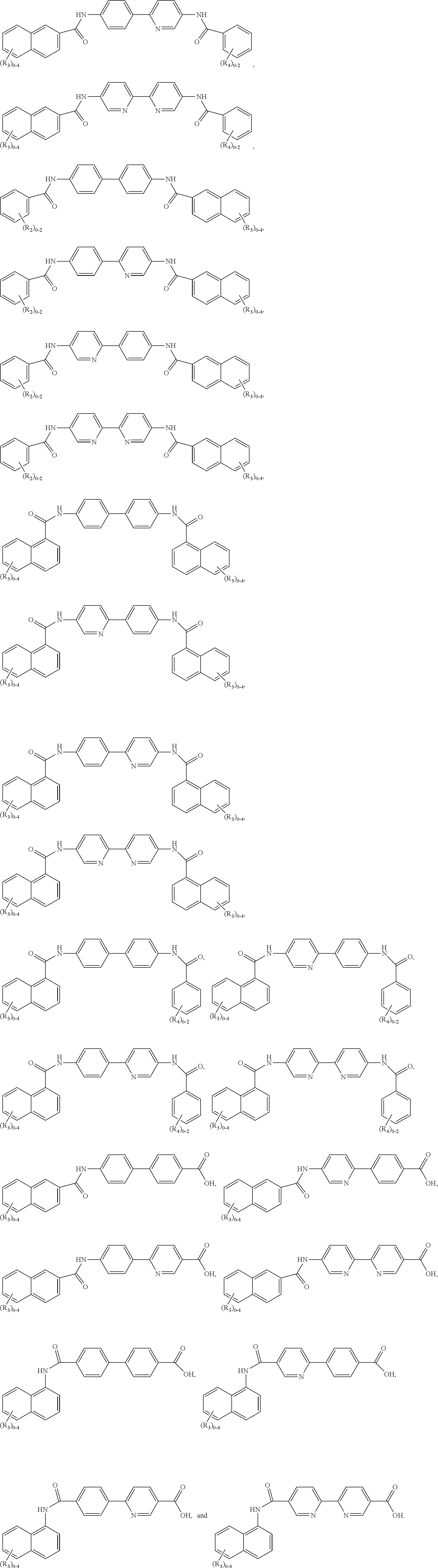

In various embodiments, the compound or salt thereof is selected from the group consisting of

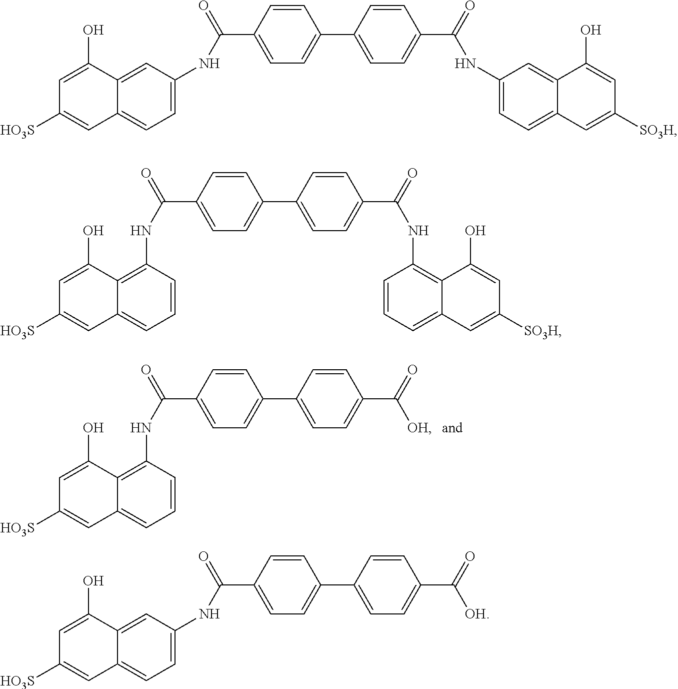

Also provided herein are pharmaceutical compositions comprising the compound or salt of the disclosure and a pharmaceutically acceptable carrier.

Also provided are uses of the compound, salt, or composition of the disclosure as a medicament in a mammal. In various embodiments, the medicament treats a viral infection. In various embodiments, the viral infection is caused by a coronavirus. In various embodiments, the coronavirus is selected from the group consisting of SARS-CoV, SARS-CoV-2, HCoV-NL63, MERS-CoV, HCoV-229E, HCoV-OC43, HCoV-HKU1, and a combination thereof. In various embodiments, the use inhibits an interaction between a coronavirus spike protein and a receptor thereof, thereby decreasing viral attachment and entry into a host cell. In embodiments, the receptor is angiotensin converting enzyme 2 (ACE2), dipeptidyl peptidase 4 (DPP4), or CD13.

Also provided are methods of preventing or treating a viral infection in a subject in need thereof, the method comprising administering to the subject a therapeutically effective amount of a compound or salt of Formula (I):

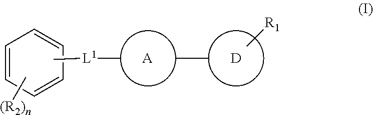

wherein Ring A is

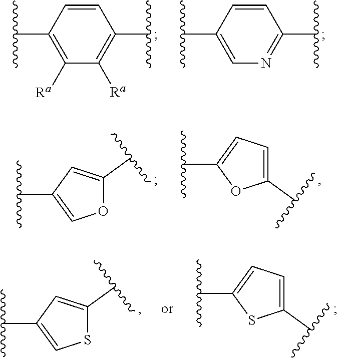

### Ring D is

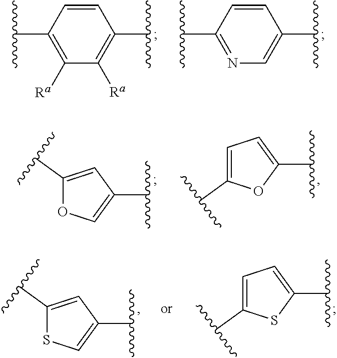

R₁ is H, halo, CF₃, SO₃H, CO₂R^{b}, NO₂, NH₂, or

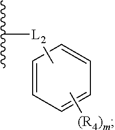

each of L₁ and L₂ independently is

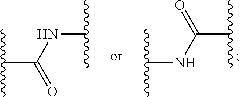 [SMILES: *NC(*)=O ; *NC(*)=O]

n is 0, 1, 2, 3, or 4; m is 0, 1, 2, 3, or 4; each R₂ independently is halo, CF₃, SO₃H, CO₂R_{b}, NO₂, or NH₂; and when two R₂ are adjacent, they can together form —(N═N—NH)— or, with the carbon atoms to which they are attached, form a C₆ aryl optionally substituted with 1-4 R₃; each R₃ independently is halo, OH, CF₃, SO₃H, CO₂R^{b}, NO₂, or NH₂; and when two R₃ are adjacent, they can together form —(N═N—NH)—; each R₄ independently is halo, CF₃, SO₃H, CO₂R^{b}, NO₂, or NH₂; and when two R₄ are adjacent, they can together form —(N═N—NH)— or, with the carbon atoms to which they are attached, form a C₆ aryl optionally substituted with 1-4 R₃; each R^{a} independently is H, C₁₋₅ alkyl, or C₁₋₅ alkoxy; and each R^{b} independently is H or C₁₋₅ alkyl; or a pharmaceutically acceptable salt thereof.

In various embodiments, the viral infection is caused by a coronavirus. In various embodiments, the coronavirus is selected from the group consisting of SARS-CoV, SARS-CoV-2, HCoV-NL63, MERS-CoV, HCoV-229E, HCoV-OC43, HCoV-HKU1, and a combination thereof.

In various embodiments, ring A is

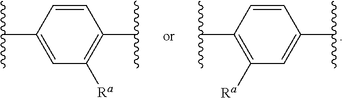

In some cases, R^{a} is H. In some cases, R^{a} is methyl.

In various embodiments, ring A is

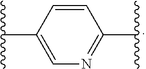 [SMILES: *c1ccc(*)nc1]

In various embodiments, ring A is

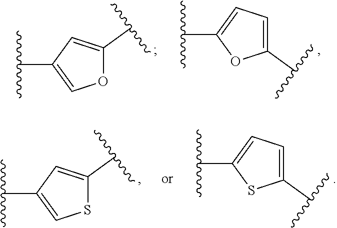 [SMILES: *c1coc(*)c1 ; *c1ccc(*)o1 ; *c1csc(*)c1 ; *c1ccc(*)s1]

In various embodiments, ring D is

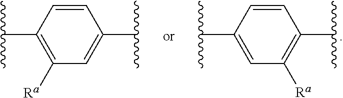

In some cases, R^{a} is H. In some cases, R^{a} is methyl.

In various embodiments, ring D is

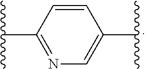 [SMILES: *c1ccc(*)nc1]

In various embodiments, ring D is

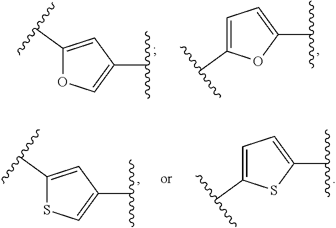 [SMILES: *c1coc(*)c1 ; *c1ccc(*)o1 ; *c1csc(*)c1 ; *c1ccc(*)s1]

In various embodiments,

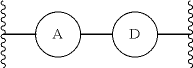

is selected from the group consisting of

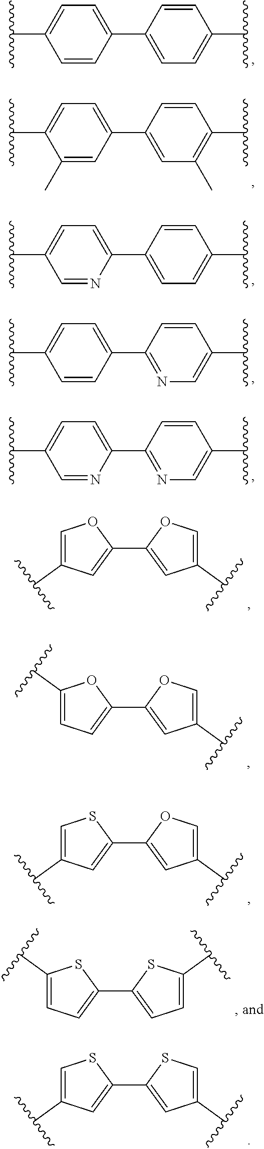

In some cases,

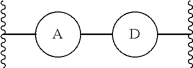

is

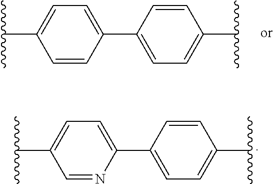 [SMILES: *c1ccc(-c2ccc(*)cc2)cc1 ; *c1ccc(-c2ccc(*)cn2)cc1]

In various embodiments, R₁ is H. In various embodiments, R₁ is halo. In some cases, R₁ is F or Cl. In various embodiments, R₁ is CF₃. In various embodiments, R₁ is SO₃H. In various embodiments, R₁ is CO₂R^{b}. In some cases, R^{b} is H. In some cases, R^{b} is C₁₋₅ alkyl. In various embodiments, R₁ is NO₂. In various embodiments, R₁ is NH₂. In various embodiments, R₁ is

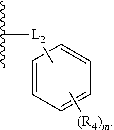

In various embodiments, L₂ is

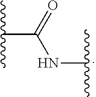 [SMILES: *NC(*)=O]

In various embodiments, L₂ is

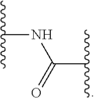 [SMILES: *NC(*)=O]

In various embodiments, m is 0. In various embodiments, m is 1, 2, 3, or 4. In various embodiments, at least one R₄ is halo. In some cases, at least one R₄ is Cl or F. In various embodiments, at least one R₄ is CF₃. In various embodiments, at least one R₄ is SO₃H. In various embodiments, at least one R₄ is CO₂R^{b}. In some cases, R^{b} is H. In some cases, R^{b} is C₁₋₅ alkyl. In various embodiments, at least one R₄ is NO₂. In various embodiments, at least one R₄ is NH₂. In various embodiments, m is at least 2, and two R₄ are adjacent and together with the carbon atoms to which they are attached form a C₆ aryl optionally substituted with 1-4 R₃.

In various embodiments, R₁ is selected from the group consisting of

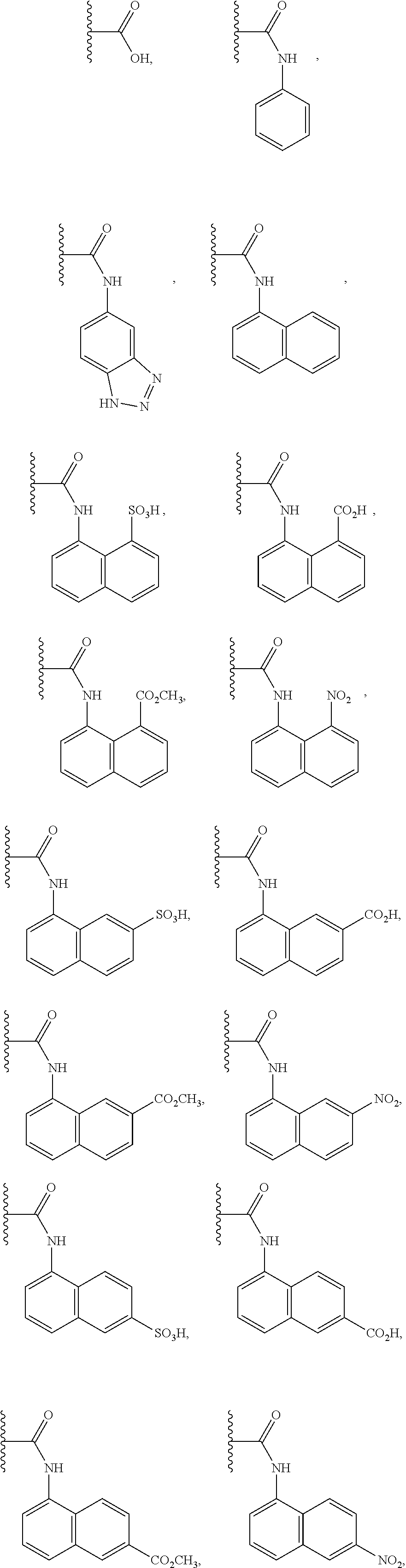

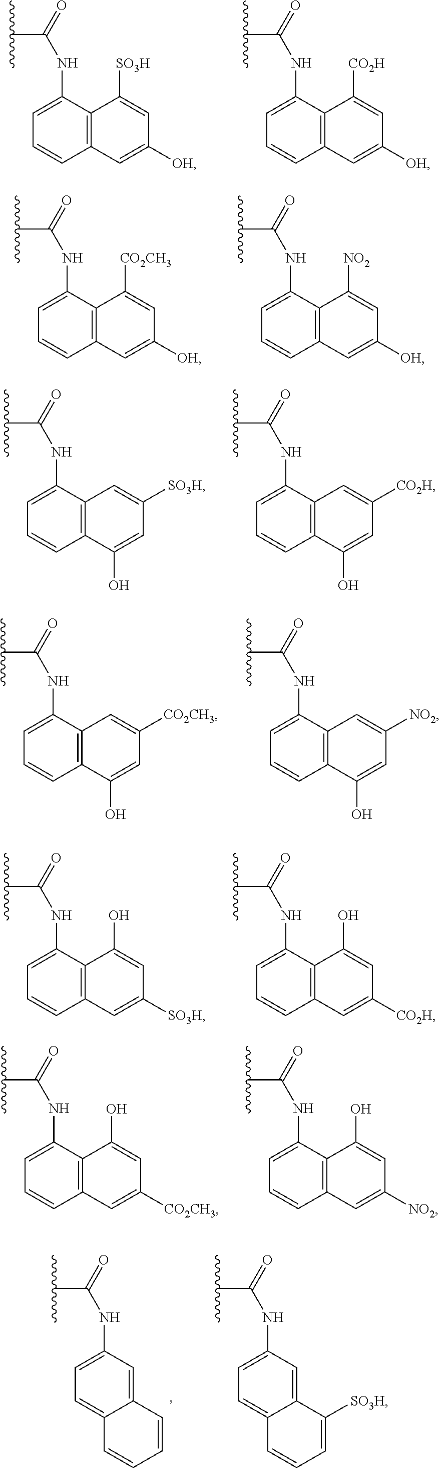

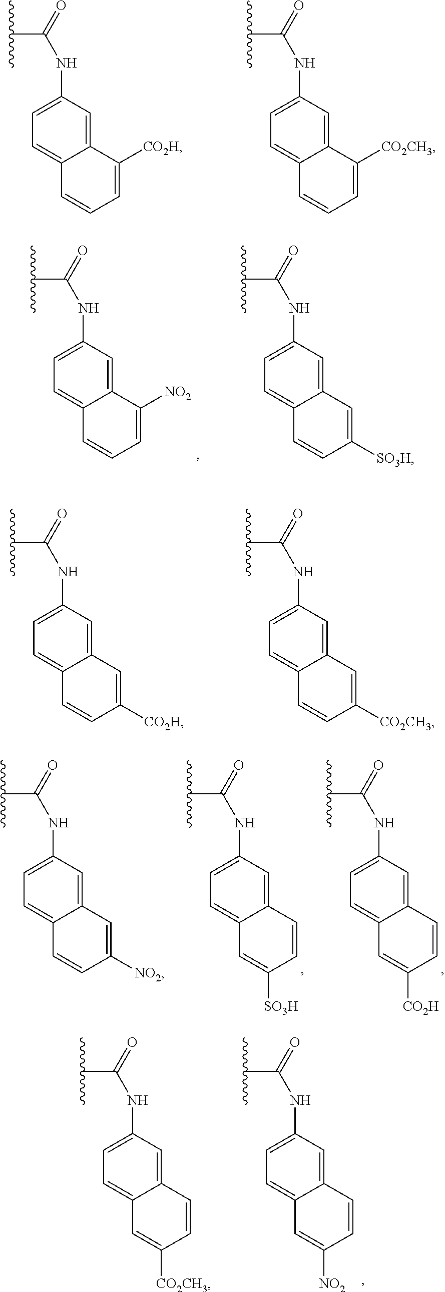

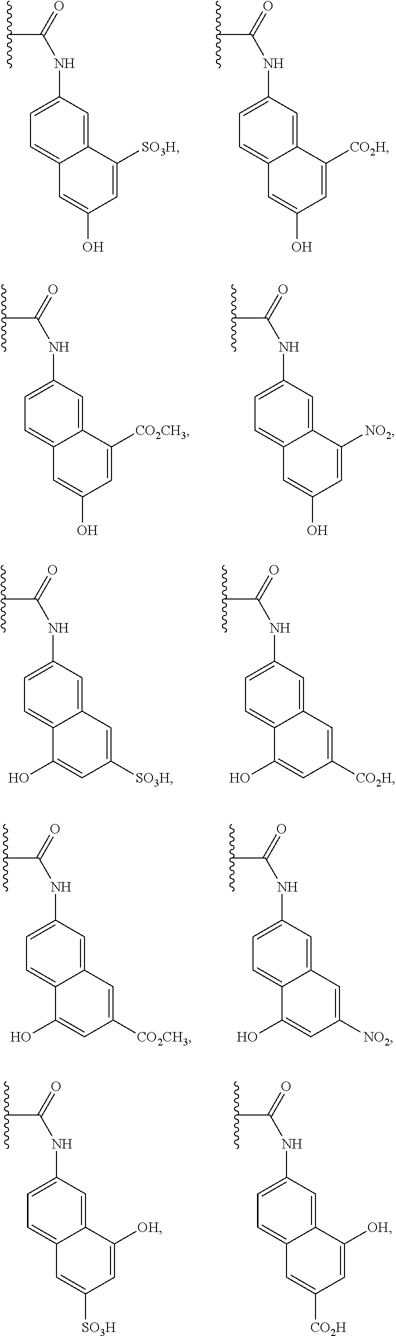

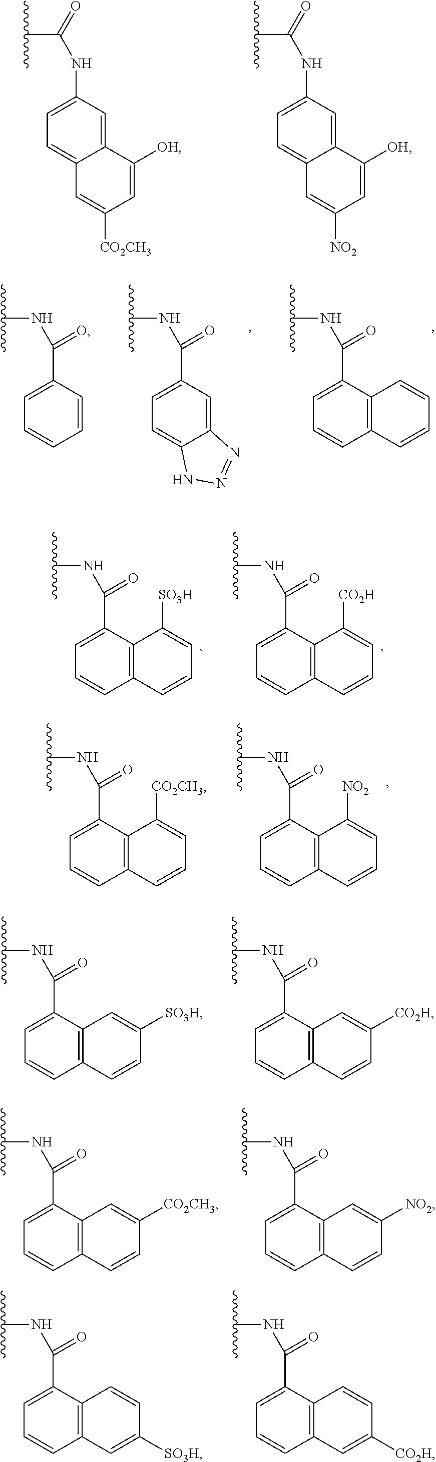

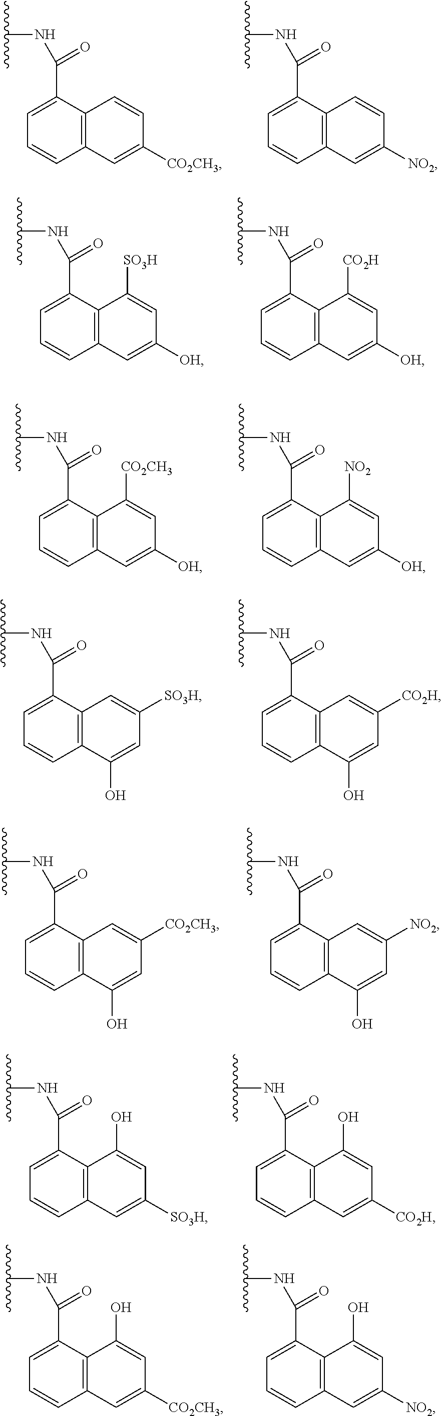

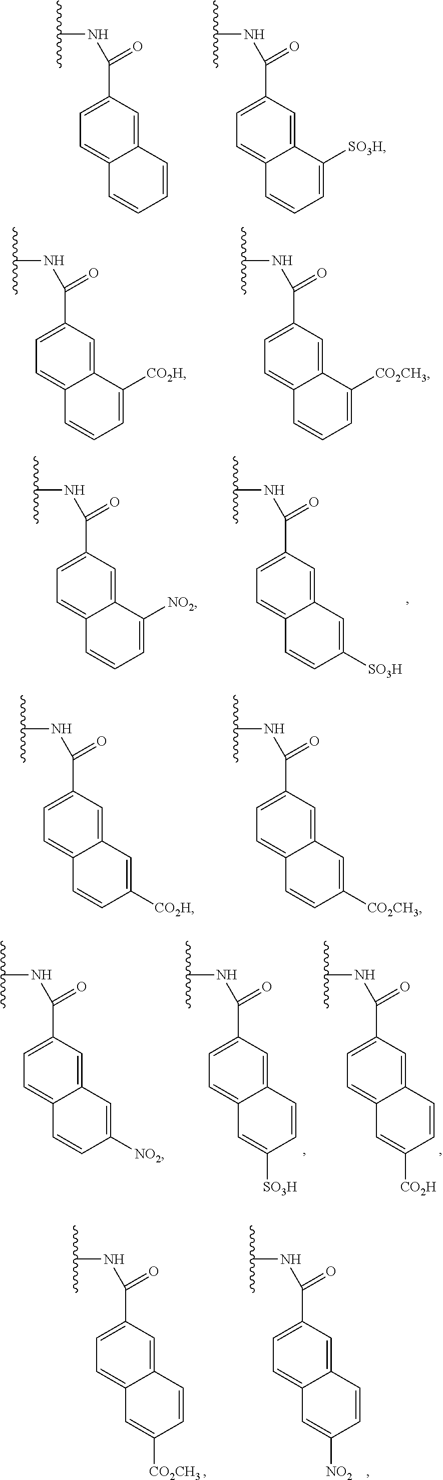

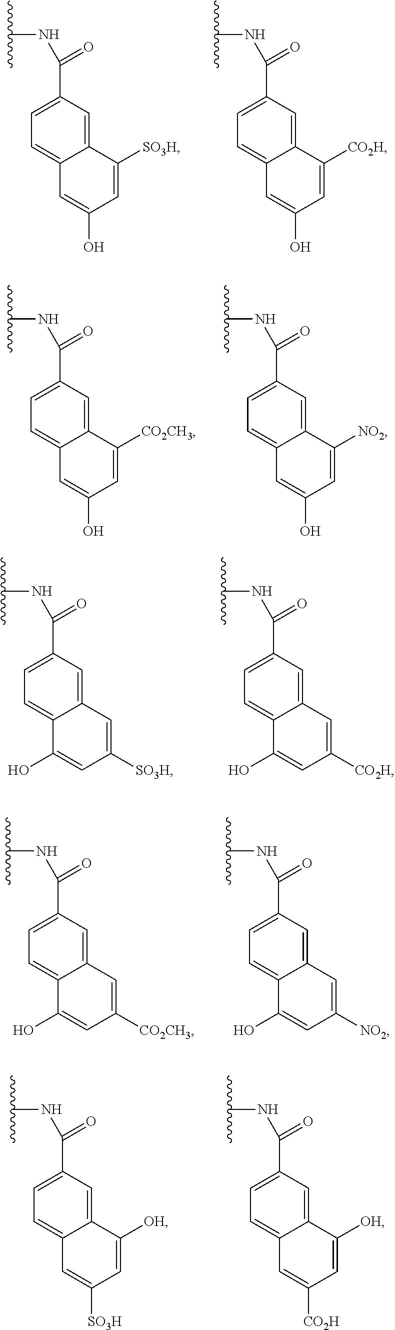

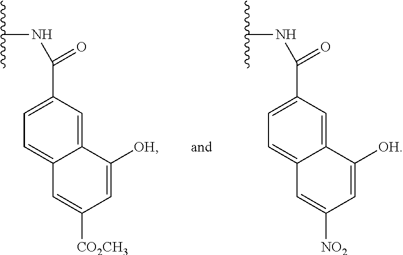 [SMILES: *NC(=O)c1ccc2cc(C(=O)OC)cc(O)c2c1 ; "and" ; *NC(=O)c1ccc2cc([N+](=O)[O-])cc(O)c2c1]

In various embodiments, L₁ is

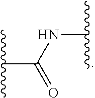 [SMILES: *NC(*)=O]

In various embodiments, L₁ is

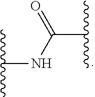 [SMILES: *NC(*)=O]

In various embodiments, n is 0. In various embodiments, n is 1, 2, 3, or 4. In various embodiments, at least one R₂ is halo. In some cases, at least one R₂ is Cl or F. In various embodiments, at least one R₂ is CF₃. In various embodiments, at least one R₂ is SO₃H. In various embodiments, at least one R₂ is CO₂R^{b}. In some cases, R^{b} is H. In some cases, R^{b} is C₁₋₅ alkyl. In various embodiments, at least one R₂ is NO₂. In various embodiments, at least one R₂ is NH₂. In various embodiments, n is at least 2, and two R₂ are adjacent and taken together with the carbon atoms to which they are attached form a C₆ aryl optionally substituted with 1-4 R₃.

In various embodiments,

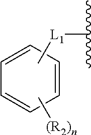

is selected from the group consisting of

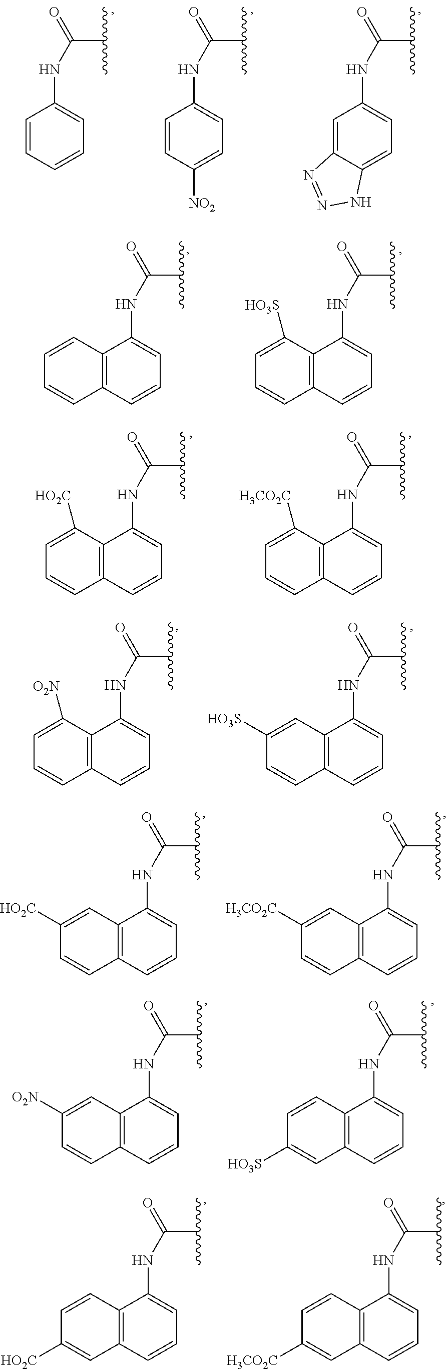

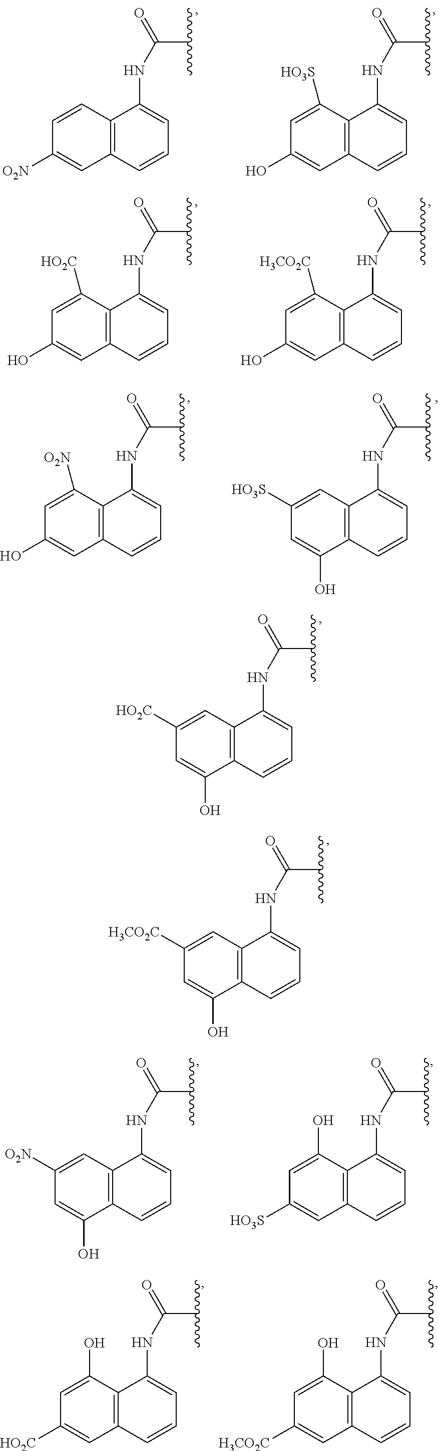

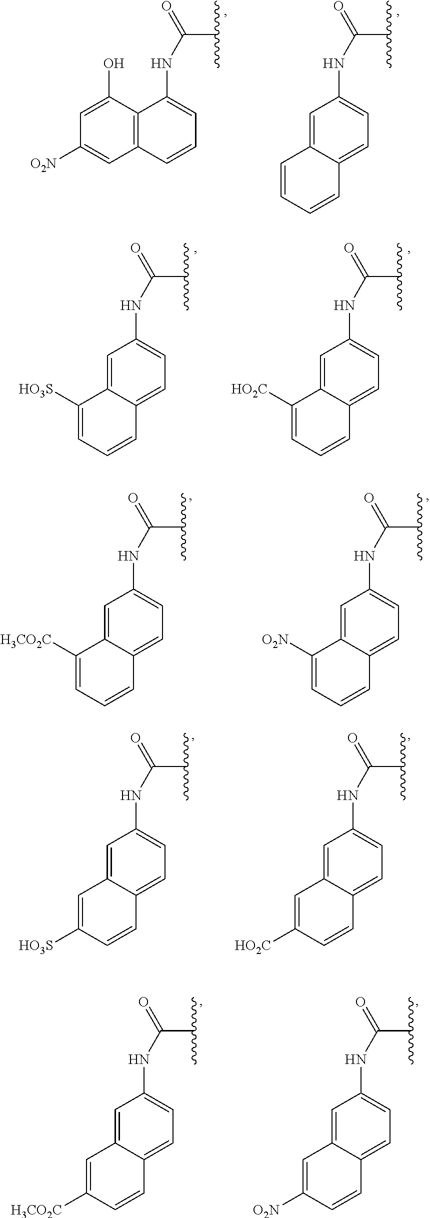

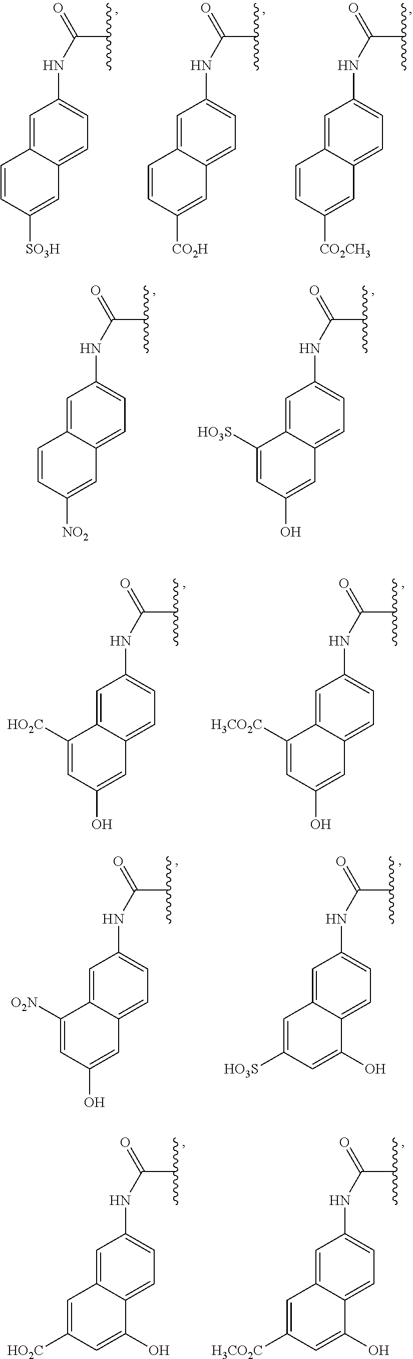

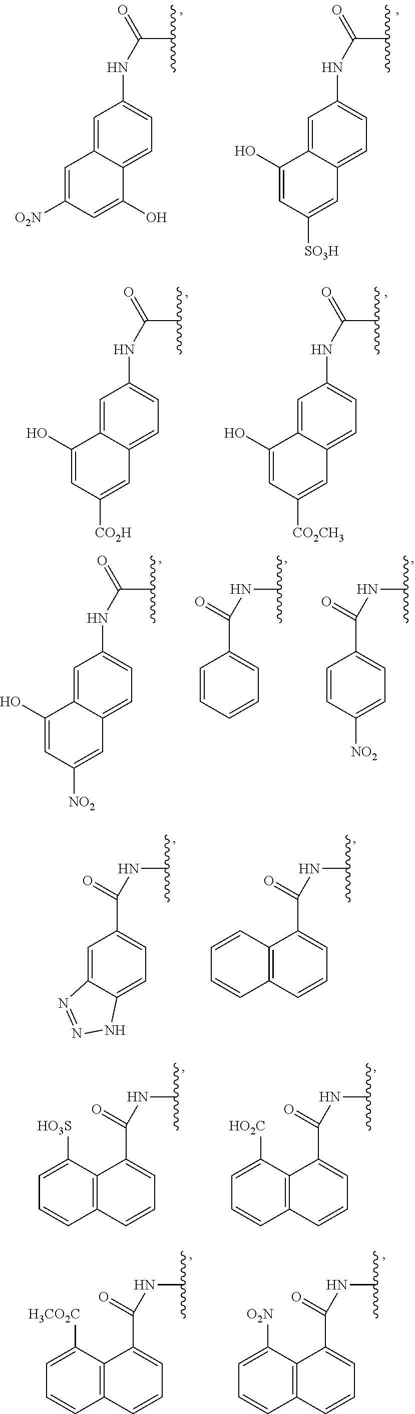

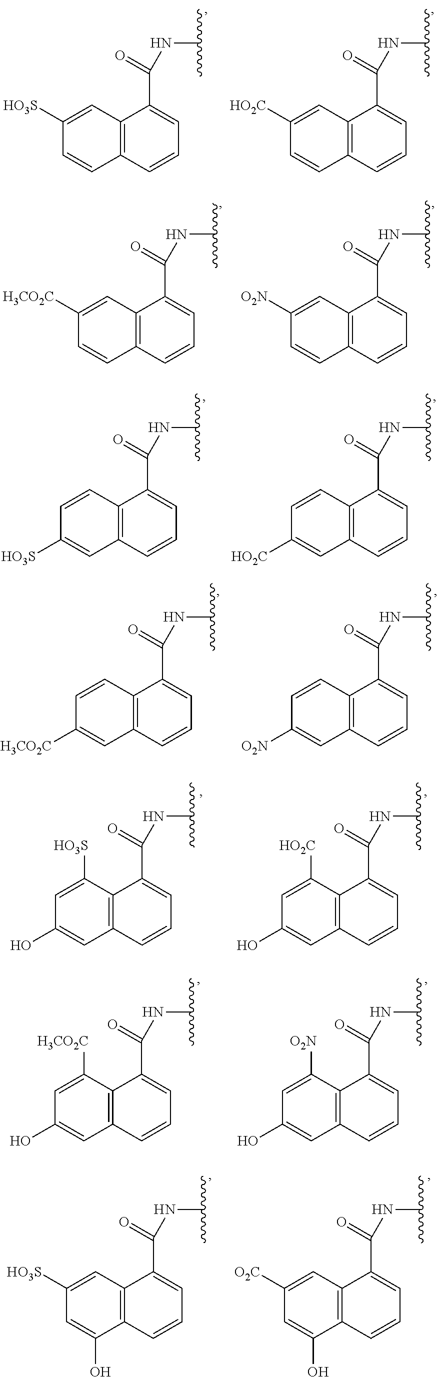

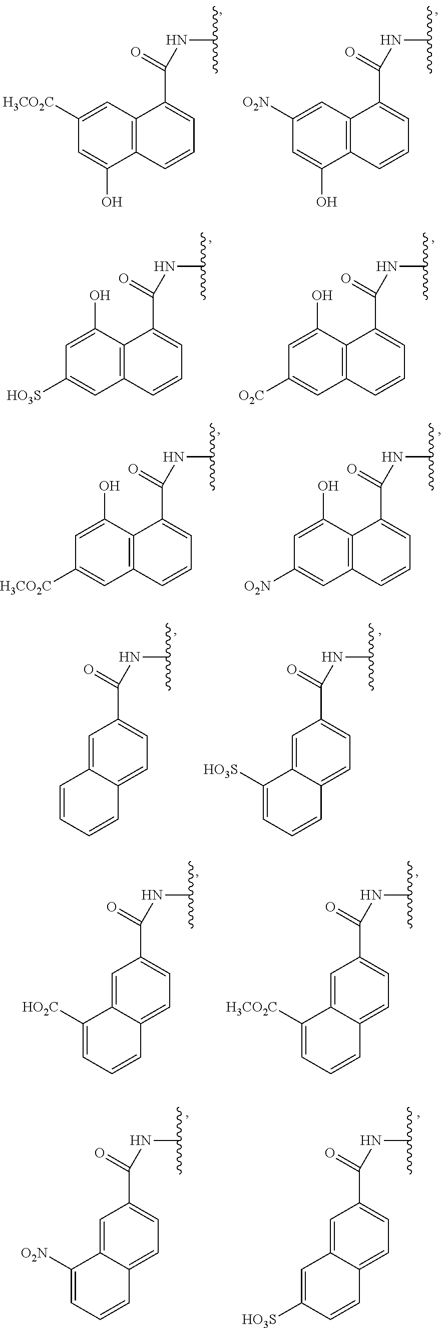

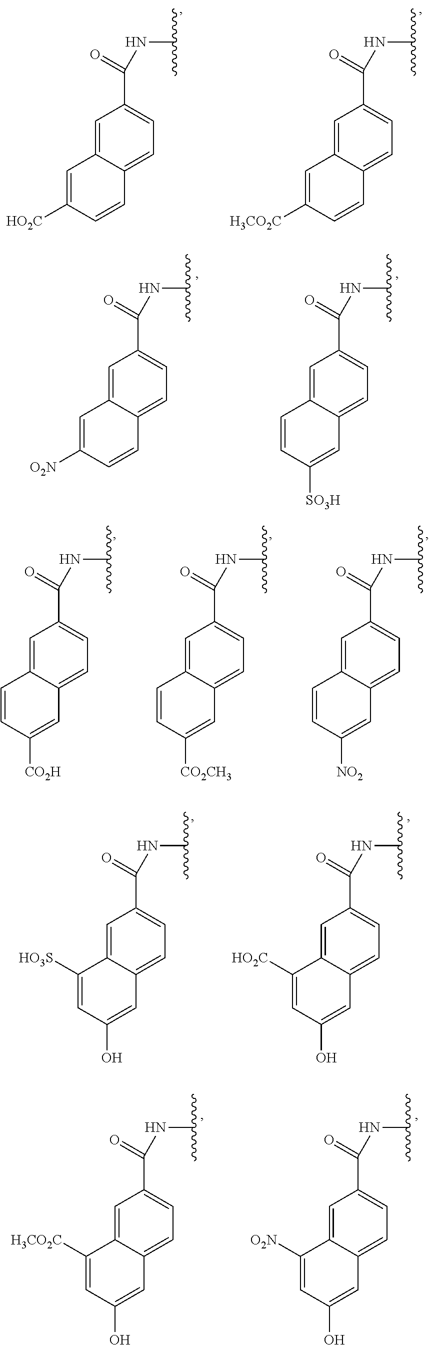

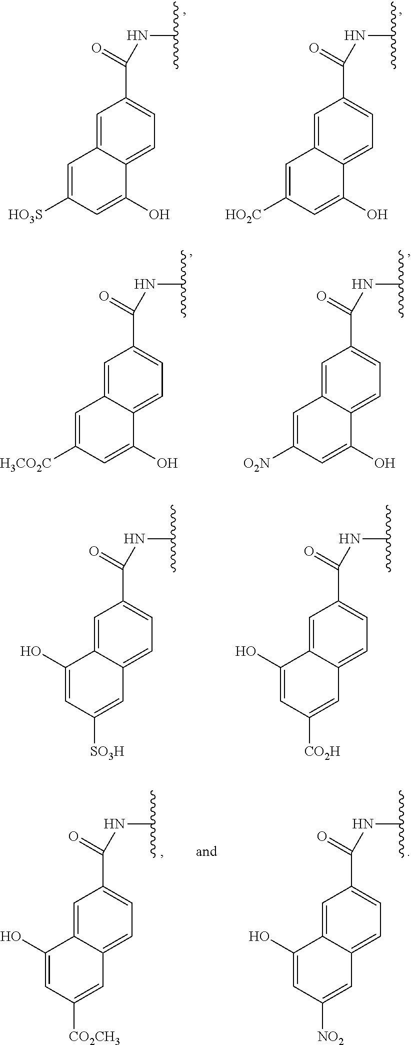

In various embodiments, the compound or salt is selected from the group consisting of

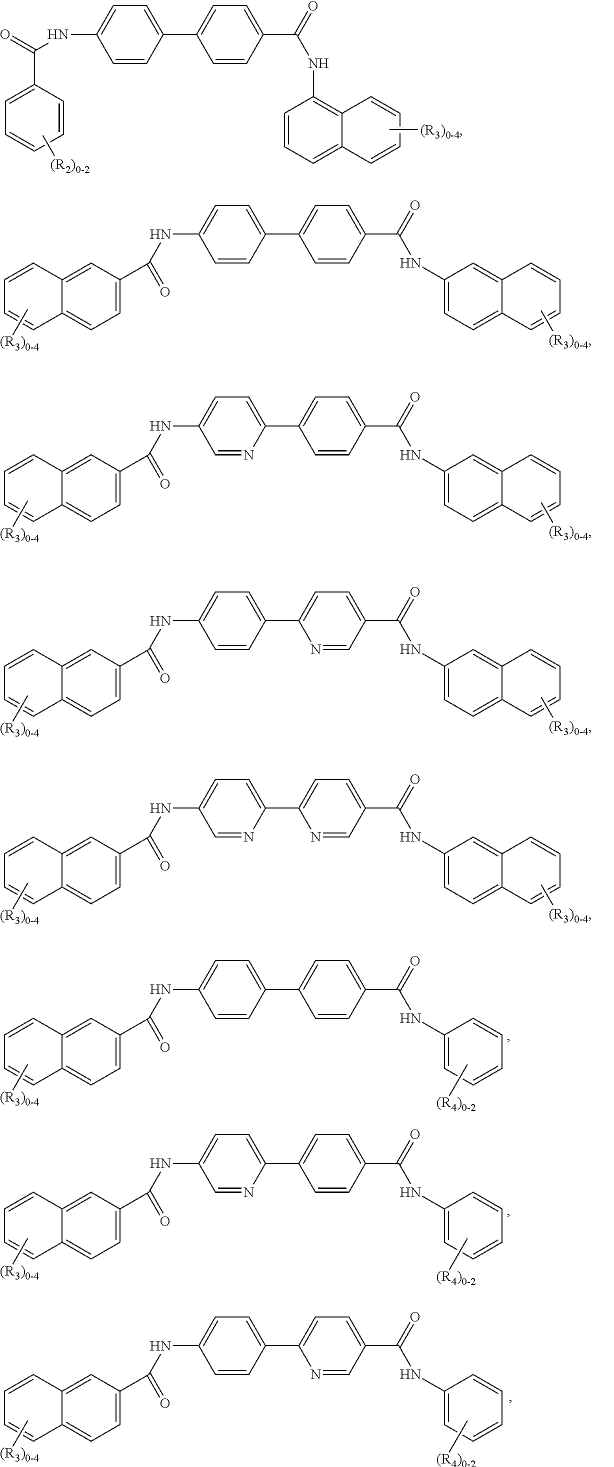

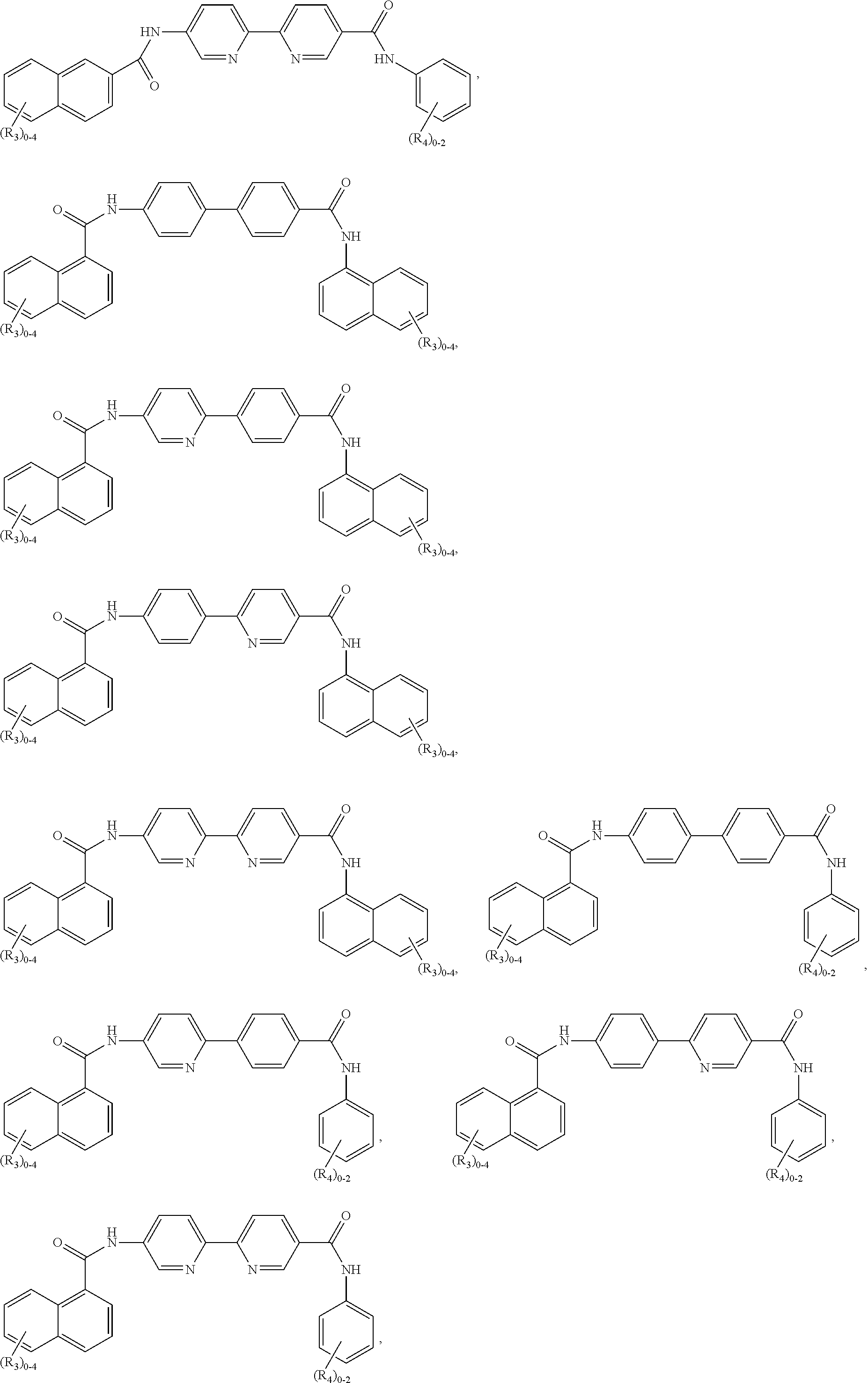

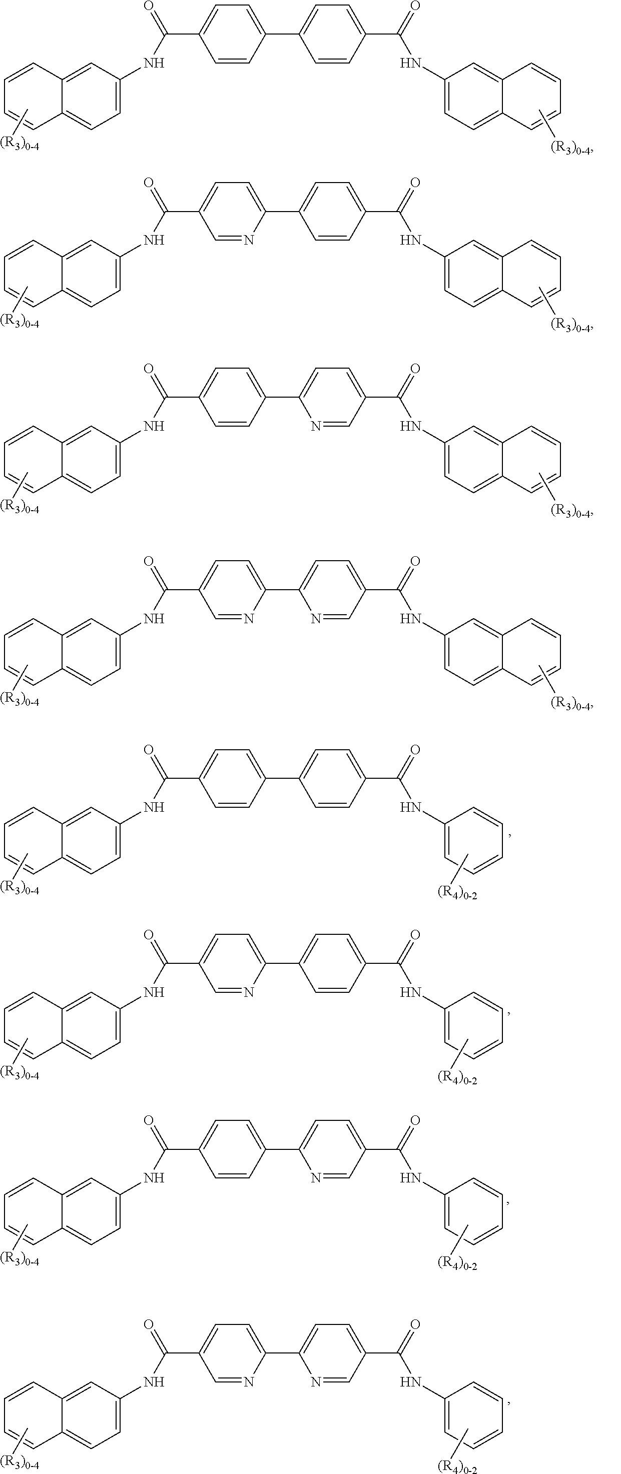

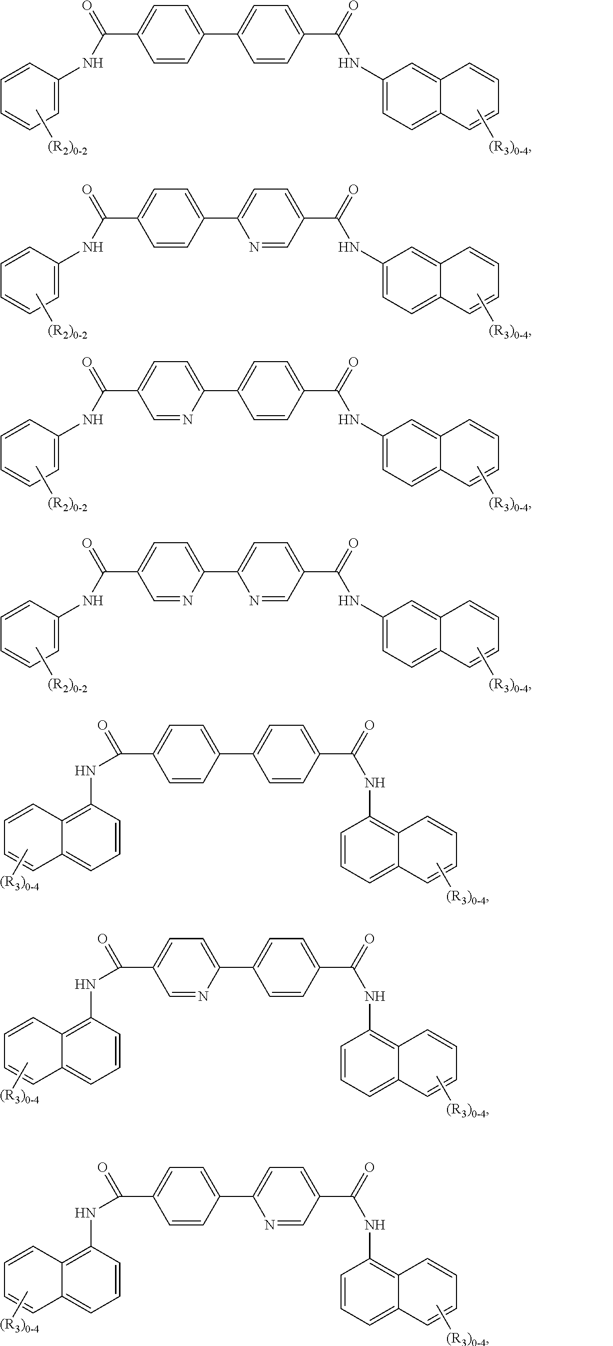

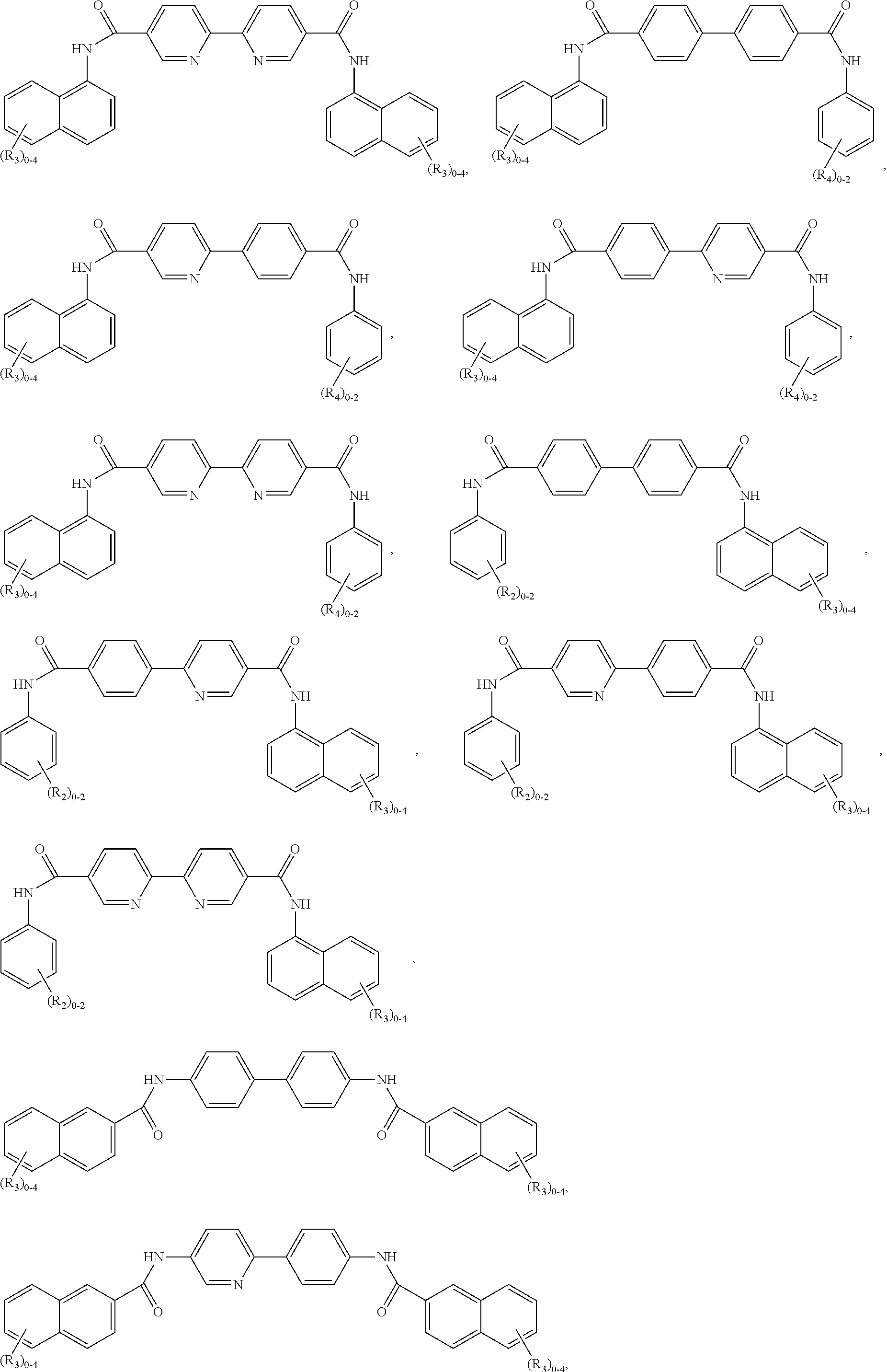

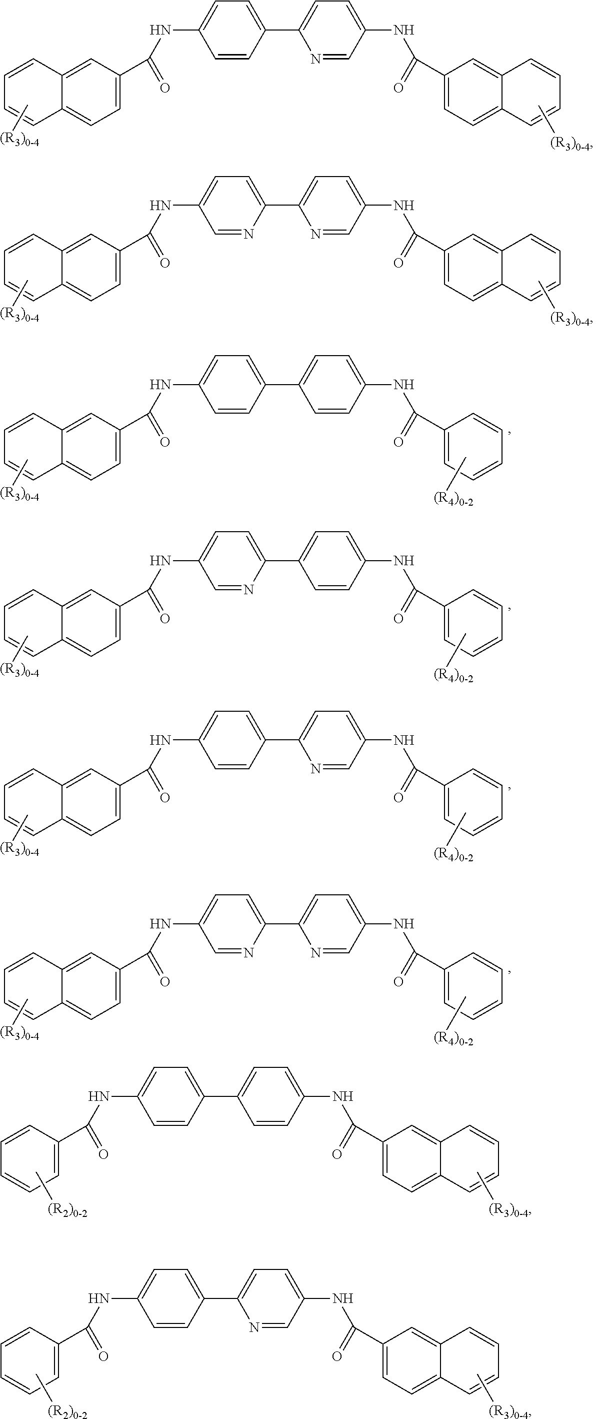

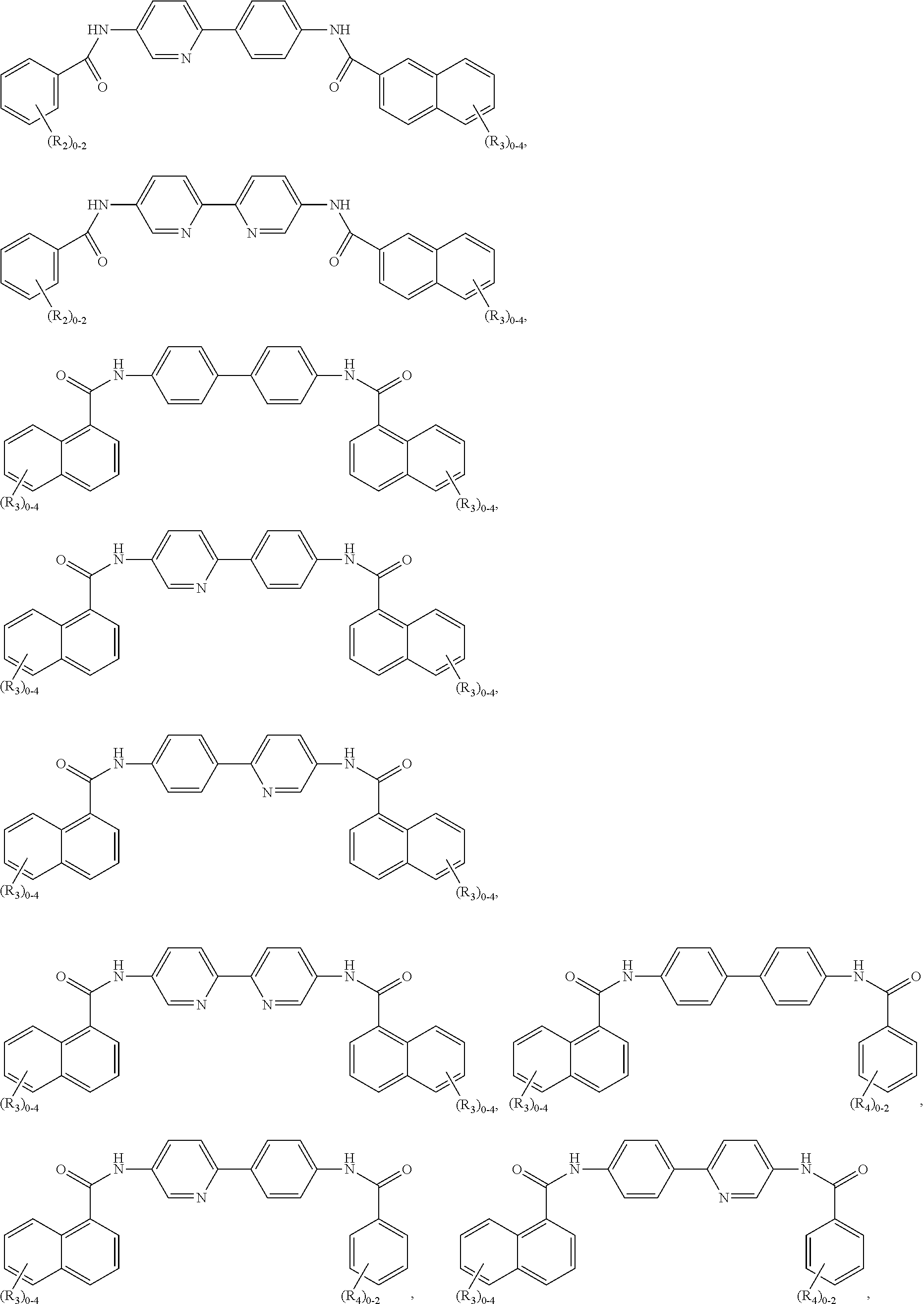

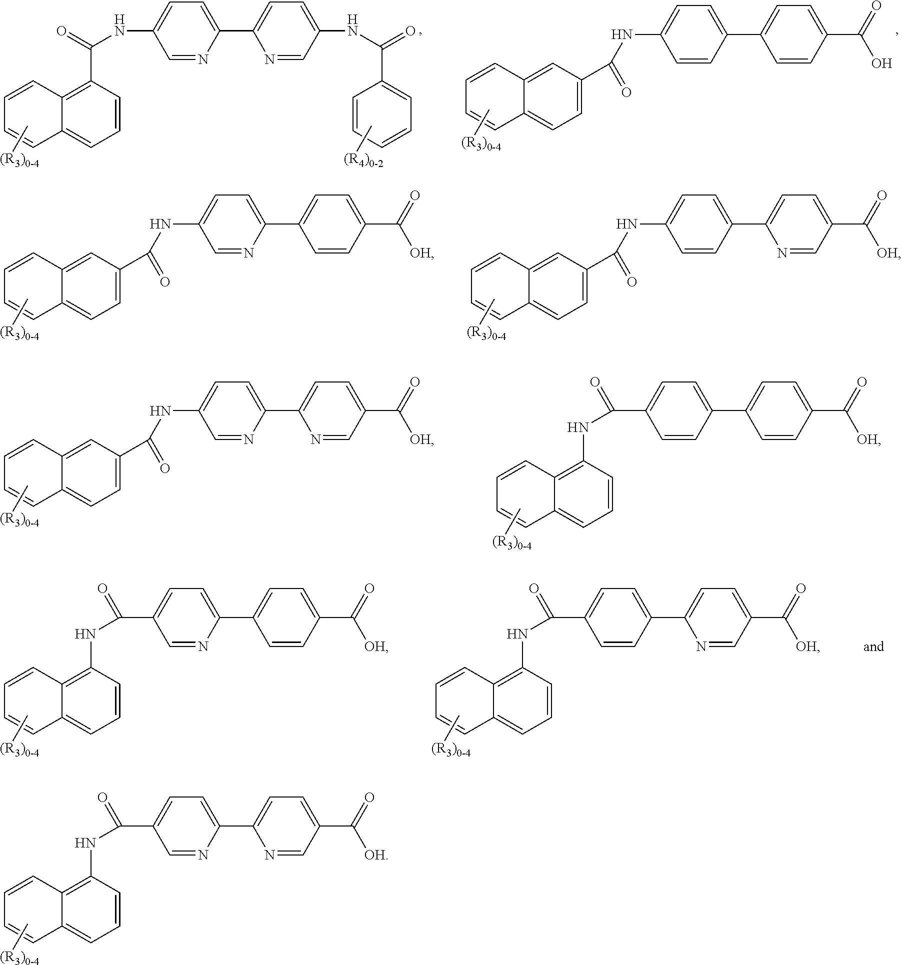

In various embodiments, the compound or salt is selected from the group consisting of

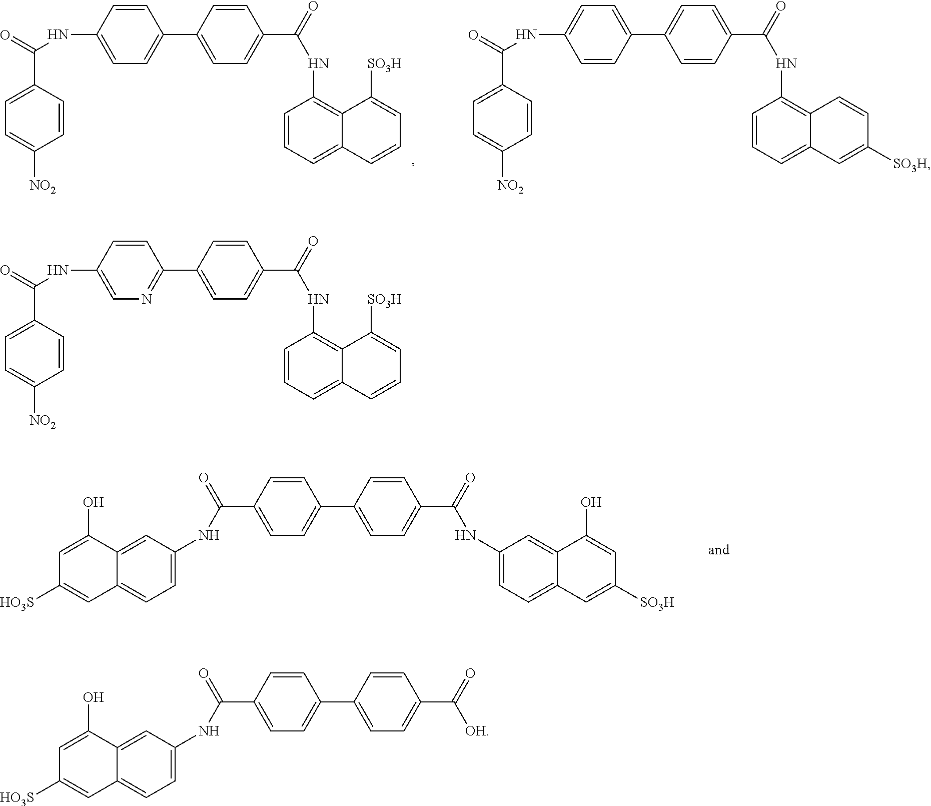 [SMILES: O=C(Nc1ccc(-c2ccc(C(=O)Nc3cccc4cccc(S(=O)(=O)O)c34)cc2)cc1)c1ccc([N+](=O)[O-])cc1 ; "," ; O=C(Nc1ccc(-c2ccc(C(=O)Nc3cccc4cc(S(=O)(=O)O)ccc34)cc2)cc1)c1ccc([N+](=O)[O-])cc1 ; O=C(Nc1ccc(-c2ccc(C(=O)Nc3cccc4cccc(S(=O)(=O)O)c34)cc2)nc1)c1ccc([N+](=O)[O-])cc1 ; O=C(Nc1ccc2cc(S(=O)(=O)O)cc(O)c2c1)c1ccc(-c2ccc(C(=O)Nc3ccc4cc(S(=O)(=O)O)cc(O)c4c3)cc2)cc1 ; O=C(O)c1ccc(-c2ccc(C(=O)Nc3ccc4cc(S(=O)(=O)O)cc(O)c4c3)cc2)cc1]

Further aspects and advantages will be apparent to those of ordinary skill in the art from a review of the following detailed description. The description hereafter includes specific embodiments with the understanding that the disclosure is illustrative and is not intended to limit the invention to the specific embodiments described herein.

### BRIEF DESCRIPTION OF THE DRAWINGS

While the specification concludes with claims particularly pointing out and distinctly claiming the subject matter that is regarded as the present disclosure, it is believed that the disclosure will be more fully understood from the following description taken in conjunction with the accompanying drawings.

FIG. 1 is a graph of concentration-response curves for binding of CoV spike proteins to human ACE2 (hACE2) in an ELISA-based assay format.

FIG. 2 is a graph of concentration-dependent inhibition of the SARS-CoV-2-RBD-hACE2 PPI by various compounds, including compounds of the disclosure.

FIG. 3 is a graph of concentration-dependent inhibition of the SARS-CoV-S1S2-hACE2 PPI by various compounds, including compounds of the disclosure.

FIG. 4 is a plot showing the results of protein thermal shift assays indicating SARS-CoV-2 RBD and not hACE2 as the binding partner for one of the compounds of the disclosure.

FIG. 5 is a plot of concentration-dependent inhibition of SARS-CoV-2 pseudovirus (BacMam) entry into ACE2 expressing host cells (HEK293T) by a compound of the disclosure.

FIG. 6 is a plot of is a plot of concentration-dependent inhibition of SARS-CoV-2 pseudovirus (VSV-AG) entry into ACE2/Furin expressing host cells (Vero-E6) by a compound of the disclosure.

### DETAILED DESCRIPTION

Provided herein are compounds that can inhibit an interaction between a coronavirus spike protein and a receptor thereof, thereby decreasing viral attachment and entry into a host cell. The compounds can be used to prevent and/or treat viral infections. In particular, provided herein are compounds of Formula (I), salts, uses, and methods of using the same. The compounds described herein have a structure of Formula (I):

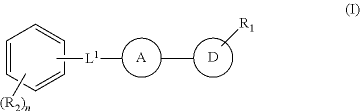

wherein the substituents are described in detail below.

The compounds or salts of the disclosure, as well as the uses and methods thereof provide several advantages, particularly as compared to antibodies or biologics. For example, the uses and methods of the disclosure can be more broadly active (e.g., less strain- and mutation sensitivity), more patient friendly (e.g., suitable for oral or inhaled administration), less immunogenic, and more controllable (shorter half-life/better biodistribution) therapies. In particular, for COVID-19, the uses and methods of the disclosure have the possibility of direct delivery into the respiratory system via inhaled or intranasal administration, which cannot be achieved for antibodies.

### Small Molecule Inhibitors of Coronavirus Attachment

Provided herein are compounds, and salts thereof, having a structure of Formula (I):

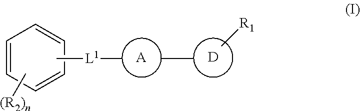

wherein
Ring A is

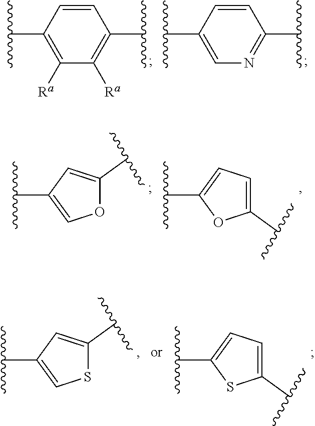

Ring D is

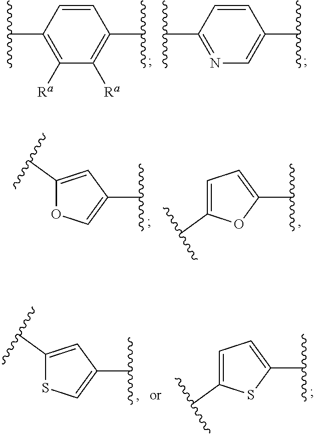

R₁ is H, halo, CF₃, SO₃H, CO₂R^{b}, NO₂, NH₂, or

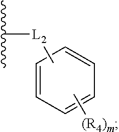

each of L₁ and L₂ independently is

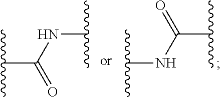 [SMILES: *NC(*)=O ; *NC(*)=O]

n is 0, 1, 2, 3, or 4;
m is 0, 1, 2, 3, or 4;
each R₂ independently is halo, CF₃, SO₃H, CO₂R^{b}, NO₂, or NH₂; and when two R₂ are adjacent, they can together form —(N═N—NH)— or, with the carbon atoms to which they are attached, form a C₆ aryl optionally substituted with 1-4 R₃;
each R₃ independently is halo, OH, CF₃, SO₃H, CO₂R^{b}, NO₂, or NH₂; and when two R₃ are adjacent, they can together form —(N═N—NH)—;
each R₄ independently is halo, CF₃, SO₃H, CO₂R^{b}, NO₂, or NH₂; and when two R₄ are adjacent, they can together form —(N═N—NH)— or, with the carbon atoms to which they are attached, form a C₆ aryl optionally substituted with 1-4 R₃;
each R^{a} independently is H, C₁₋₅ alkyl, or C₁₋₅ alkoxy; and,
each R^{b} independently is H or C₁₋₅ alkyl;
or a pharmaceutically acceptable salt thereof.

In embodiments, when (a) L₁-ring A-ring D-L₂ is

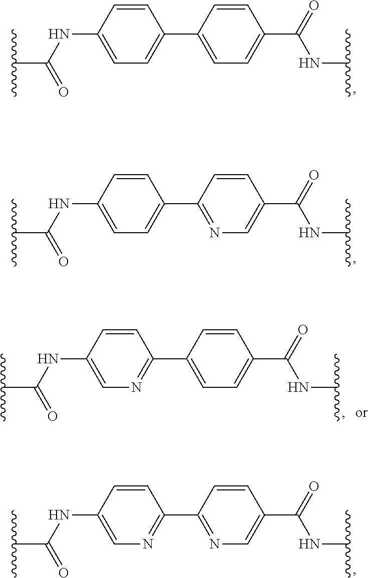 [SMILES: *NC(=O)c1ccc(-c2ccc(NC(*)=O)cc2)cc1 ; *NC(=O)c1ccc(-c2ccc(NC(*)=O)cc2)nc1 ; *NC(=O)c1ccc(-c2ccc(NC(*)=O)cn2)cc1 ; *NC(=O)c1ccc(-c2ccc(NC(*)=O)cn2)nc1]

then two adjacent R₂, with the carbon atoms to which they are attached, form a C₆ aryl, optionally substituted with 1-4 R₃; and when (b) L₁-ring A-ring D-L₂ is

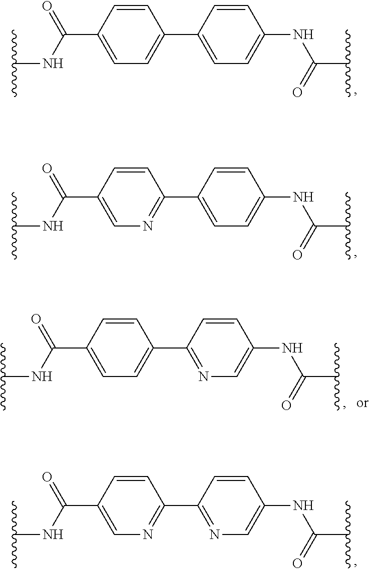 [SMILES: *NC(=O)c1ccc(-c2ccc(NC(*)=O)cc2)cc1 ; *NC(=O)c1ccc(-c2ccc(NC(*)=O)cc2)nc1 ; *NC(=O)c1ccc(-c2ccc(NC(*)=O)cn2)cc1 ; *NC(=O)c1ccc(-c2ccc(NC(*)=O)cn2)nc1]

then two adjacent R₄, with the carbon atoms to which they are attached, form a C₆ aryl, optionally substituted with 1-4 R₃.

As provided herein, ring A is

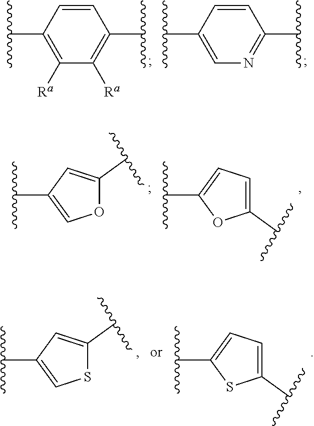

In embodiments, ring A is

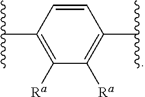

In embodiments, ring A is

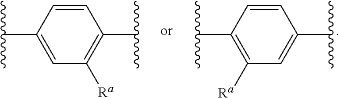

As provided herein, each R^{a} independently is H, C₁₋₅ alkyl, or C₁₋₅ alkoxy. In embodiments, each R^{a} is H (i.e., ring A is

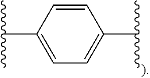 [SMILES: *c1ccc(*)cc1]

In embodiments, at least one R^{a} is C₁₋₅ alkyl. As used herein, the term “alkyl” refers to straight chained and branched saturated hydrocarbon groups. The term Cₙ means the group has “n” carbon atoms. For example, C₃ alkyl refers to an alkyl group that has 3 carbon atoms. C₁₋₅ alkyl refers to an alkyl group having a number of carbon atoms encompassing the entire range (i.e., 1 to 5 carbon atoms), as well as all subgroups (e.g., 2-5, 3-5, 1-4, 2-4, 3-4, 1, 2, 3, 4, and 5 carbon atoms). Nonlimiting examples of alkyl groups include methyl, ethyl, n-propyl, isopropyl, n-butyl, sec-butyl (2-methylpropyl), tert-butyl (1,1-dimethylethyl), and n-pentyl. In embodiments, at least one R^{a} is methyl (e.g., ring A is

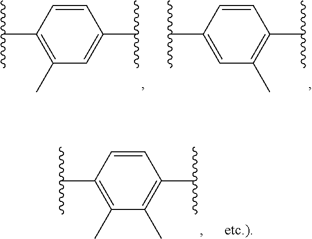

In embodiments, at least one R^{a} is C₁₋₅ alkoxy. As used herein, the term “alkoxy” refers to an “—O-alkyl” group, such as methoxy, ethoxy, propoxy, butoxy, and pentoxy. In embodiments, at least one R^{a} is methoxy (e.g., ring A is

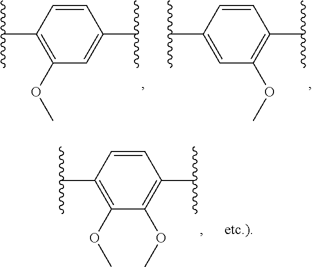

In embodiments, one R^{a} is C₁₋₅ alkyl and the other is C₁₋₅ alkoxy. In embodiments, ring A is

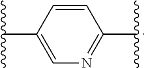 [SMILES: *c1ccc(*)nc1]

In embodiments, ring A is

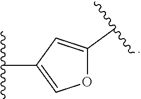 [SMILES: *c1coc(*)c1]

In embodiments, ring A is

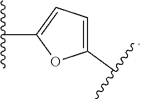 [SMILES: *c1ccc(*)o1]

In embodiments, ring A is

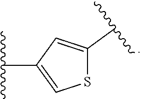 [SMILES: *c1csc(*)c1]

In embodiments, ring A is

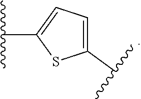 [SMILES: *c1ccc(*)s1]

In embodiments, ring A is

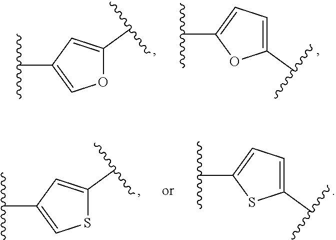 [SMILES: *c1coc(*)c1 ; *c1ccc(*)o1 ; *c1csc(*)c1 ; *c1ccc(*)s1]

As provided herein, ring D is

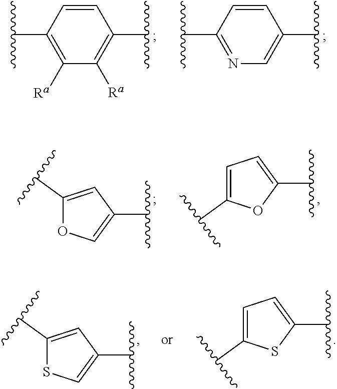

In embodiments, ring D is

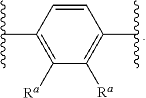

In embodiments, ring D is

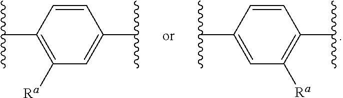

As provided herein, each R^{a} independently is H, C₁₋₅ alkyl, or C₁₋₅ alkoxy. In embodiments, each R^{a} is H (i.e., ring D is

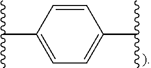 [SMILES: *c1ccc(*)cc1]

In embodiments, at least one R^{a} is C₁₋₅ alkyl. In embodiments, at least one R^{a} is methyl (e.g., ring D is

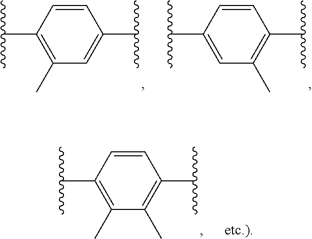

In embodiments, at least one R^{a} is C₁₋₅ alkoxy. In embodiments, at least one R^{a} is methoxy (e.g., ring D is

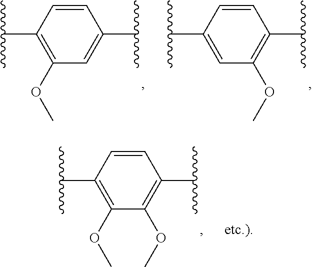

In embodiments, one R^{a} is C₁₋₅ alkyl and the other is C₁₋₅ alkoxy. In embodiments, ring D is

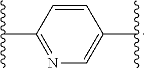 [SMILES: *c1ccc(*)nc1]

In embodiments, ring D is

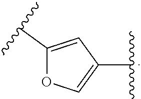 [SMILES: *c1coc(*)c1]

In embodiments, ring D is

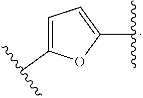 [SMILES: *c1ccc(*)o1]

In embodiments, ring D is

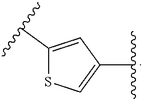 [SMILES: *c1csc(*)c1]

In embodiments, ring D is

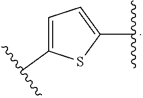 [SMILES: *c1ccc(*)s1]

In embodiments, ring D is

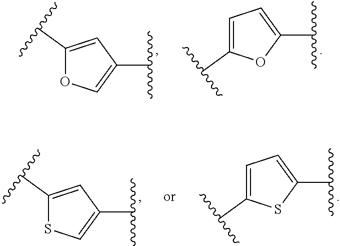 [SMILES: *c1coc(*)c1 ; *c1ccc(*)o1 ; *c1csc(*)c1 ; *c1ccc(*)s1]

In embodiments,

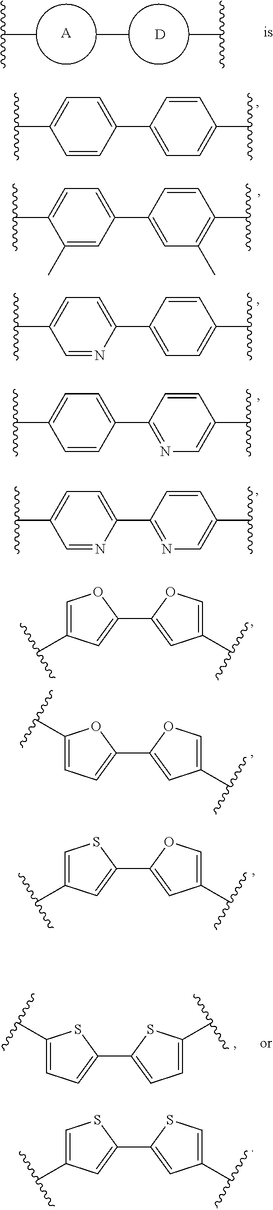

In embodiments,

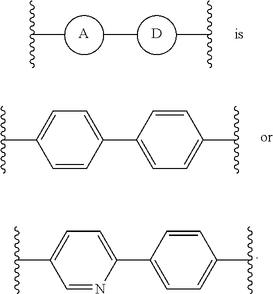

As provided herein, R₁ is H, halo, CF₃, SO₃H, CO₂R^{b}, NO₂, NH₂, or

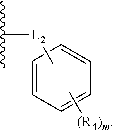

In embodiments, R₁ is H. In embodiments, R₁ is halo. For example, R₁ can be Cl or F. In embodiments, R₁ is CF₃. In embodiments, R₁ is SO₃H. In embodiments, R₁ is CO₂R^{b}. As provided herein, each R^{b} independently is H or C₁₋₅ alkyl. For example, R₁ can be CO₂H, or CO₂Me. In embodiments, R₁ is NO₂. In embodiments, R₁ is NH₂. In embodiments, R₁ is

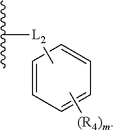

As provided herein, L₂ is

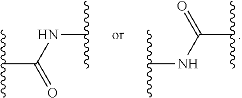 [SMILES: *NC(*)=O ; *NC(*)=O]

In embodiments, L₂ is

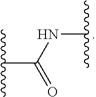 [SMILES: *NC(*)=O]

In embodiments, L₂ is

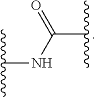 [SMILES: *NC(*)=O]

As provided herein, m is 0, 1, 2, 3, or 4. In embodiments, m is 0. In embodiments, m is 1. In embodiments, m is 2. In embodiments, m is 3. In embodiments, m is 4.

As provided herein, in embodiments wherein m is at least 1, each R₄ independently is halo, CF₃, SO₃H, CO₂R^{b}, NO₂, or NH₂; and when two R₄ are adjacent, they can together form —(N═N—NH)— or, with the carbon atoms to which they are attached, form a C₆ aryl optionally substituted with 1-4 R₃. In embodiments, at least one R₄ is halo. For example, at least one R₄ can be F or Cl. In embodiments, at least one R₄ is CF₃. In embodiments, at least one R₄ is SO₃H. In embodiments, at least one R₄ is CO₂R^{b}. For example, at least one R₄ can be CO₂H or CO₂Me. In embodiments, at least one R₄ is NO₂. In embodiments, at least one R₄ is NH₂. In embodiments, two adjacent R₄ together form —(N═N—NH)—. In embodiments, two adjacent R₄ together with the carbon atoms to which they are attached form a C₆ aryl, optionally substituted with 1-4 R₃.

As provided herein, each R₃ independently is halo, OH, CF₃, SO₃H, CO₂R^{b}, NO₂, or NH₂; and when two R₃ are adjacent, they can together form —(N═N—NH)—. In embodiments, at least one R₃ is halo. For example, at least one R₃ can be F or Cl. In embodiments, at least one R₃ is OH. In embodiments, at least one R₃ is CF₃. In embodiments, at least one R₃ is SO₃H. In embodiments, at least one R₃ is CO₂R^{b}. For example, at least one R₃ can be CO₂H or CO₂Me.

In embodiments, at least one R₃ is NO₂. In embodiments, at least one R₃ is NH₂. In embodiments, two adjacent R₃ together form —(N═N—NH)—. In embodiments, two adjacent R₄ together with the carbon atoms to which they are attached form a C₆ aryl substituted with SO₃H. In embodiments, two adjacent R₄ together with the carbon atoms to which they are attached form a C₆ aryl substituted with CO₂H. In embodiments, two adjacent R₄ together with the carbon atoms to which they are attached form a C₆ aryl substituted with CO₂Me. In embodiments, two adjacent R₄ together with the carbon atoms to which they are attached form a C₆ aryl substituted with NO₂. In embodiments, two adjacent R₄ together with the carbon atoms to which they are attached form a C₆ aryl substituted with OH and SO₃H. In embodiments, two adjacent R₄ together with the carbon atoms to which they are attached form a C₆ aryl substituted with CO₂H and OH. In embodiments, two adjacent R₄ together with the carbon atoms to which they are attached form a C₆ aryl substituted with CO₂Me and OH. In embodiments, two adjacent R₄ together with the carbon atoms to which they are attached form a C₆ aryl substituted with NO₂ and OH.

In embodiments, R₁ is

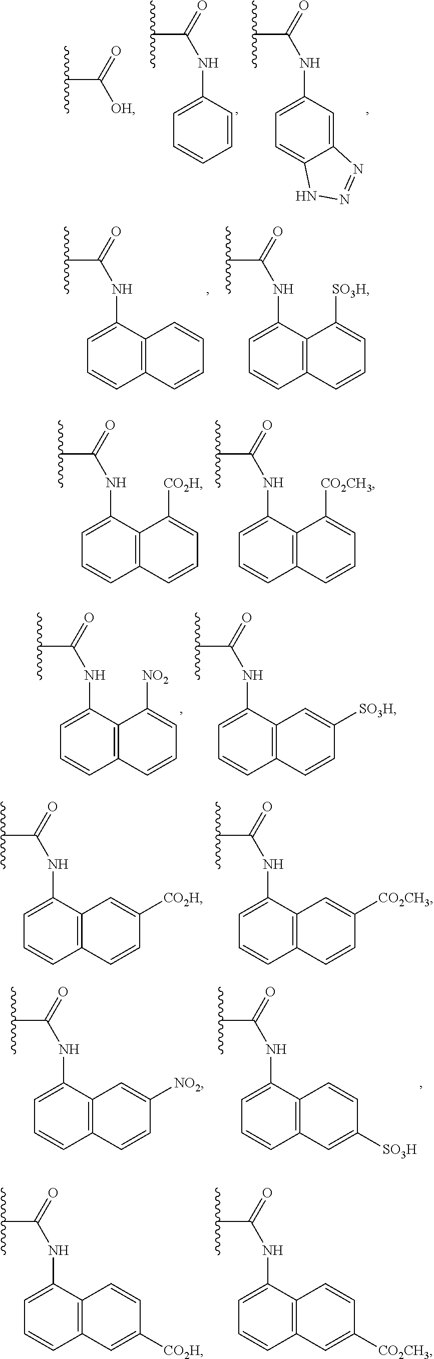

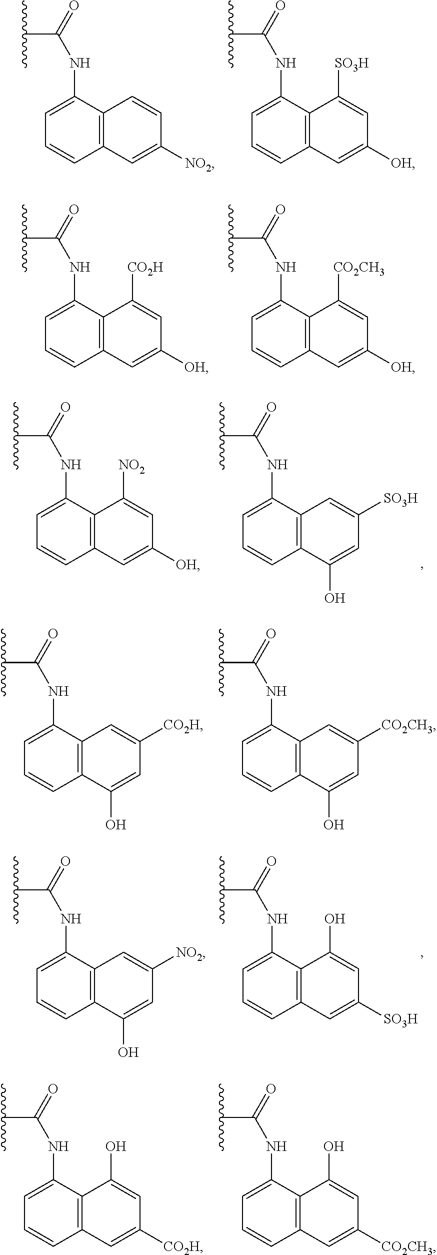

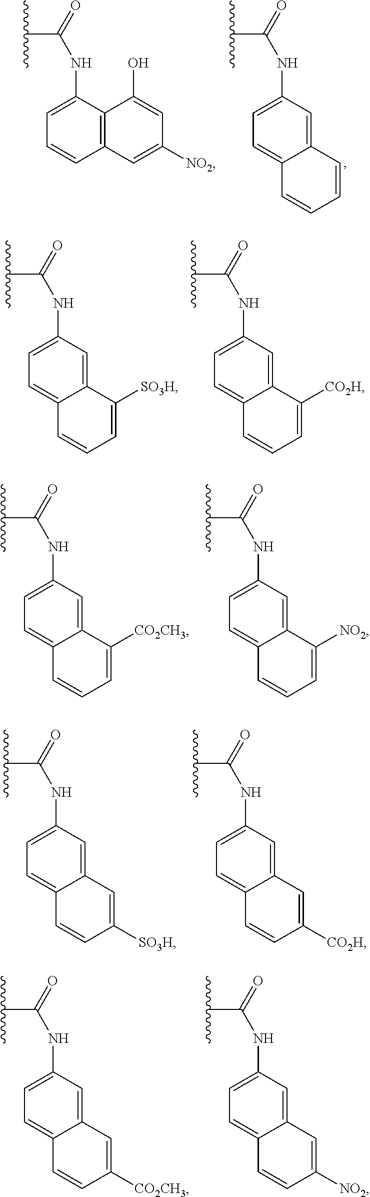

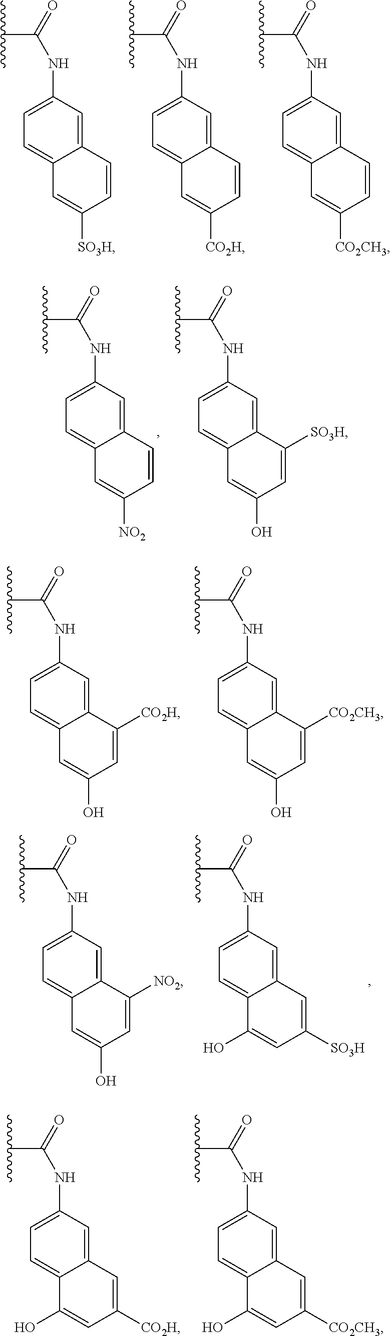

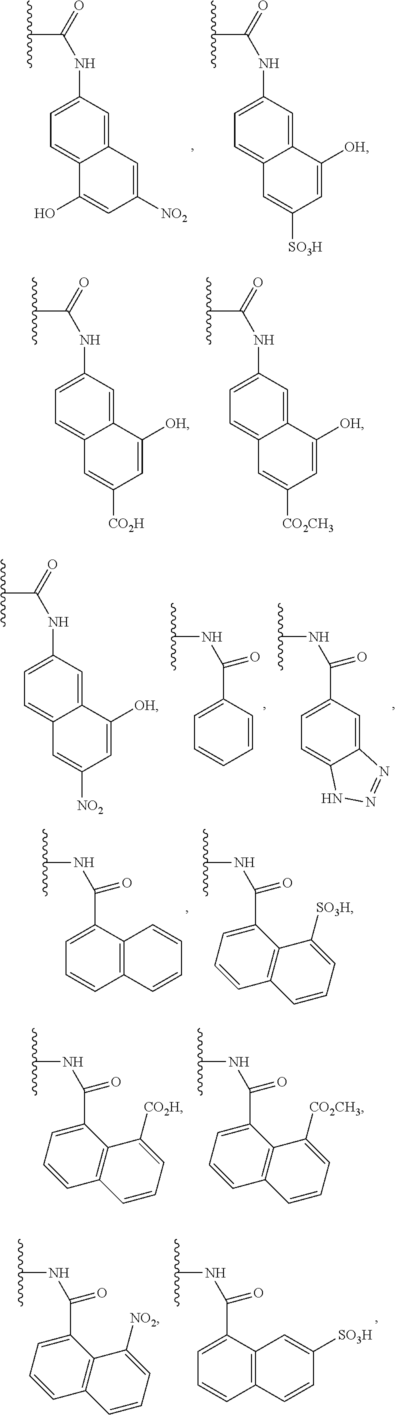

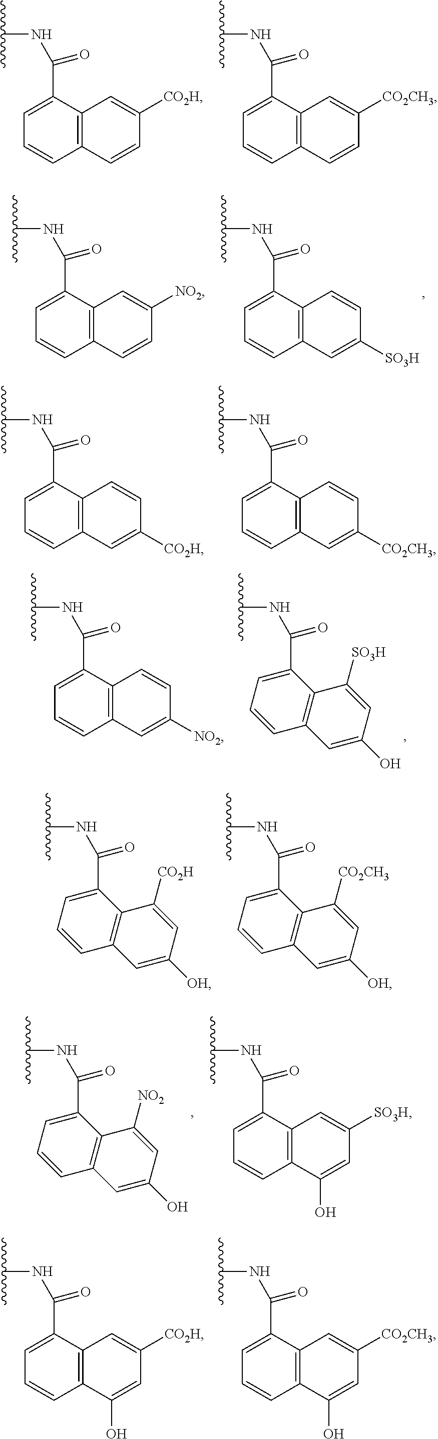

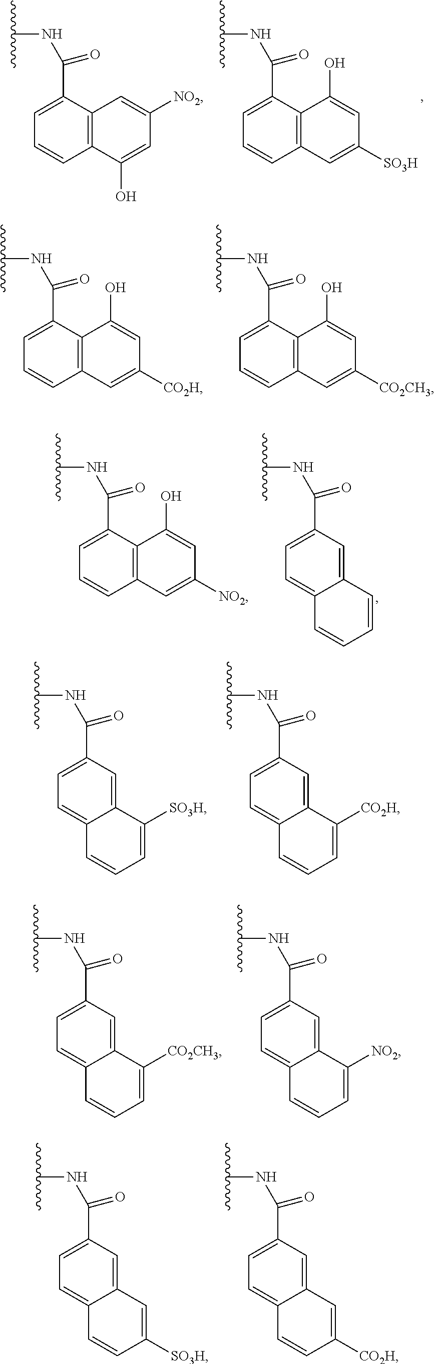

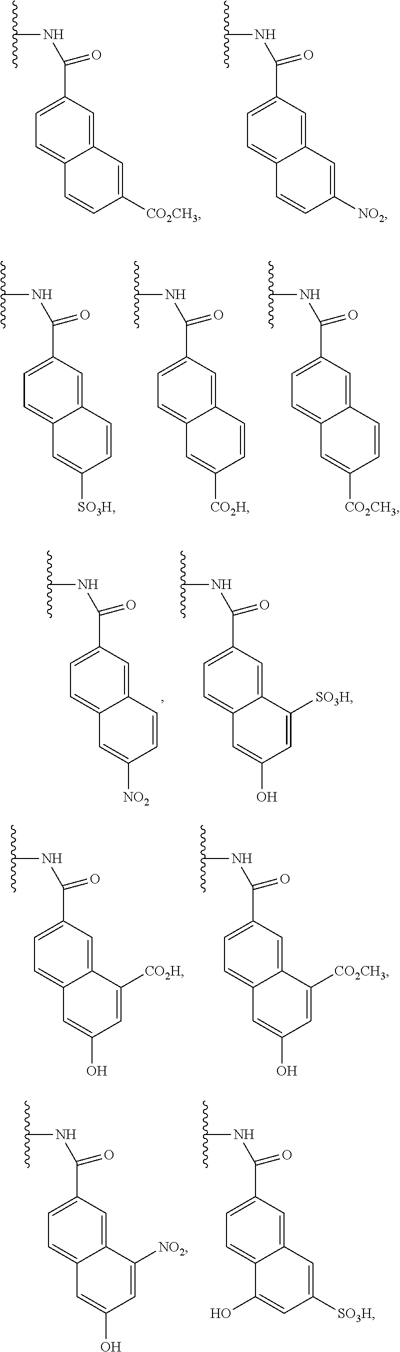

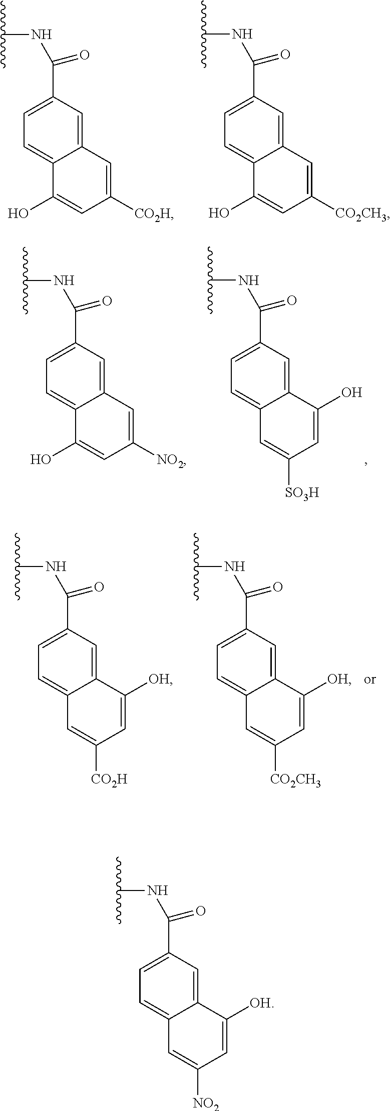

As provided herein, L₁ is

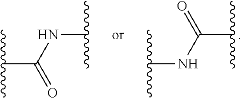 [SMILES: *NC(*)=O ; *NC(*)=O]

In embodiments, L₁ is

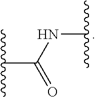 [SMILES: *NC(*)=O]

In embodiments, L₁ is

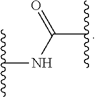 [SMILES: *NC(*)=O]

As provided herein, n is 0, 1, 2, 3, or 4. In embodiments, n is 0. In embodiments, n is 1. In embodiments, n is 2. In embodiments, n is 3. In embodiments, n is 4.

As provided herein, when n is at least 1, each R₂ independently is halo, CF₃, SO₃H, CO₂R^{b}, NO₂, or NH₂; and when two R₂ are adjacent, they can together form —(N═N—NH)— or, with the carbon atoms to which they are attached, form a C₆ aryl optionally substituted with 1-4 R₃. In embodiments, at least one R₂ is halo. For example, at least one R₂ can be F or Cl. In embodiments, at least one R₂ is CF₃. In embodiments, at least one R₂ is SO₃H. In embodiments, at least one R₂ is CO₂R^{b}. For example, at least one R₂ can be CO₂H or CO₂Me. In embodiments, at least one R₂ is NO₂. In embodiments, at least one R₂ is NH₂. In embodiments, two adjacent R₂ together form —(N═N—NH)—. In embodiments, two adjacent R₂ together with the carbon atoms to which they are attached form a C₆ aryl, optionally substituted with 1-4 R₃.

Each R₃ can be as described herein. In embodiments, two adjacent R₂ together with the carbon atoms to which they are attached form a C₆ aryl substituted with SO₃H. In embodiments, two adjacent R₂ together with the carbon atoms to which they are attached form a C₆ aryl substituted with CO₂H. In embodiments, two adjacent R₂ together with the carbon atoms to which they are attached form a C₆ aryl substituted with CO₂Me. In embodiments, two adjacent R₂ together with the carbon atoms to which they are attached form a C₆ aryl substituted with NO₂. In embodiments, two adjacent R₂ together with the carbon atoms to which they are attached form a C₆ aryl substituted with OH and SO₃H. In embodiments, two adjacent R₂ together with the carbon atoms to which they are attached form a C₆ aryl substituted with CO₂H and OH. In embodiments, two adjacent R₂ together with the carbon atoms to which they are attached form a C₆ aryl substituted with CO₂Me and OH. In embodiments, two adjacent R₂ together with the carbon atoms to which they are attached form a C₆ aryl substituted with NO₂ and OH.

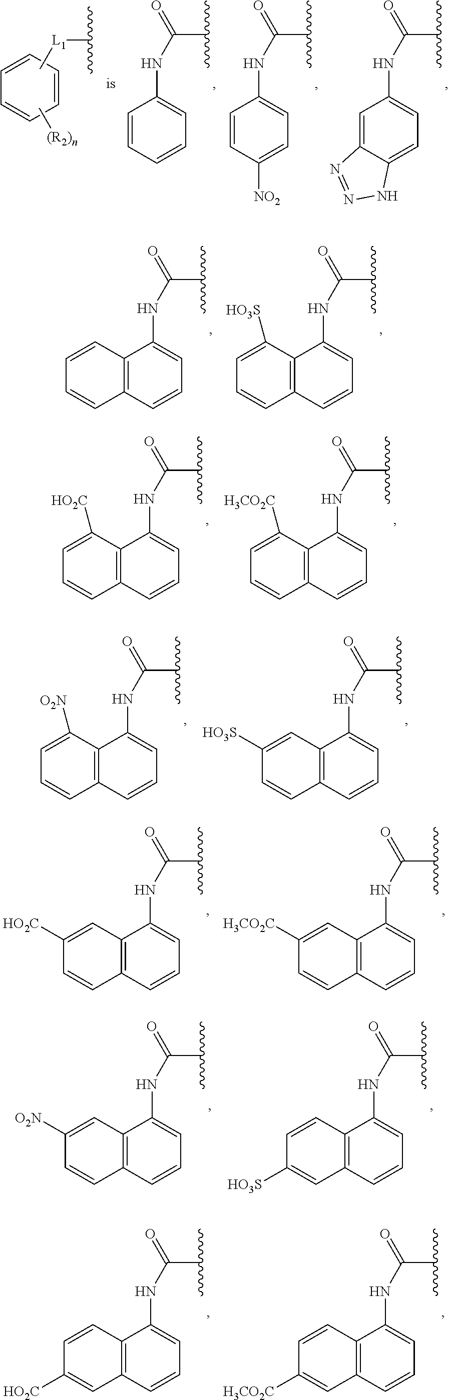

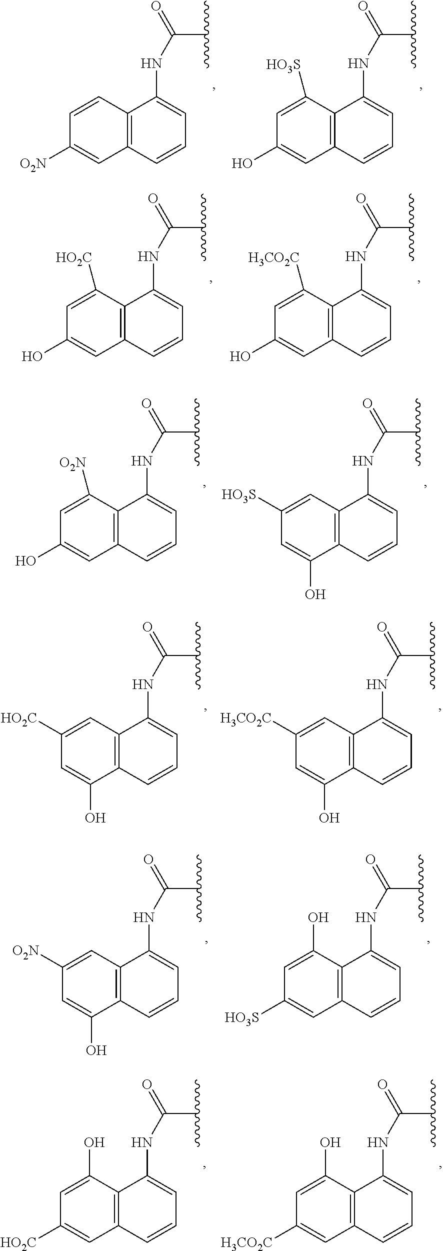

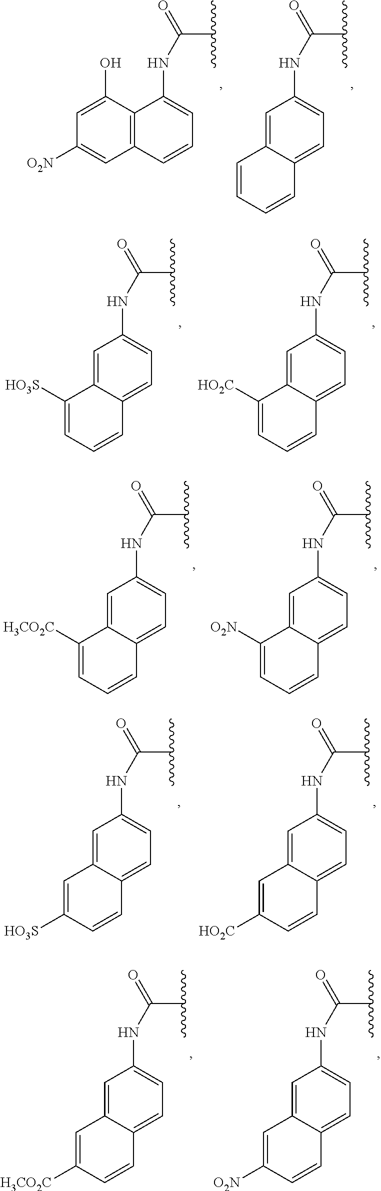

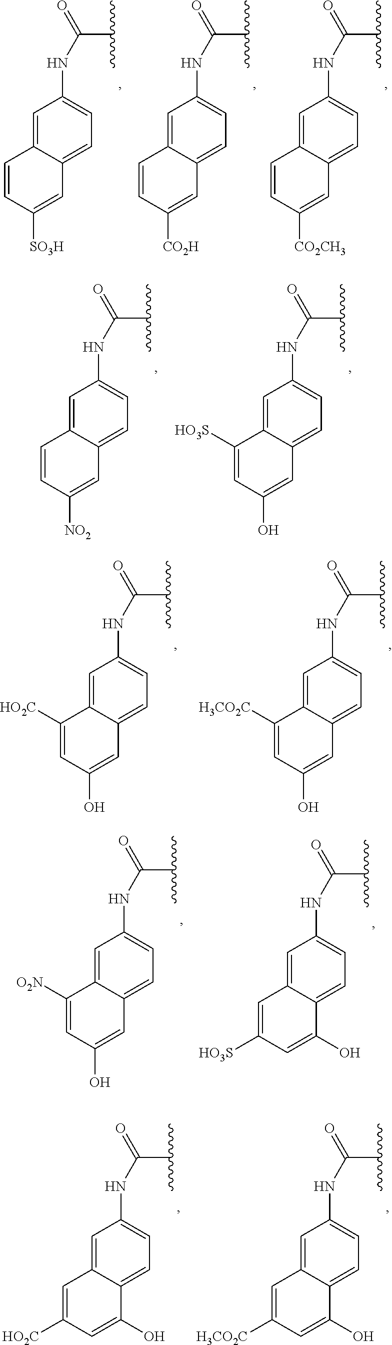

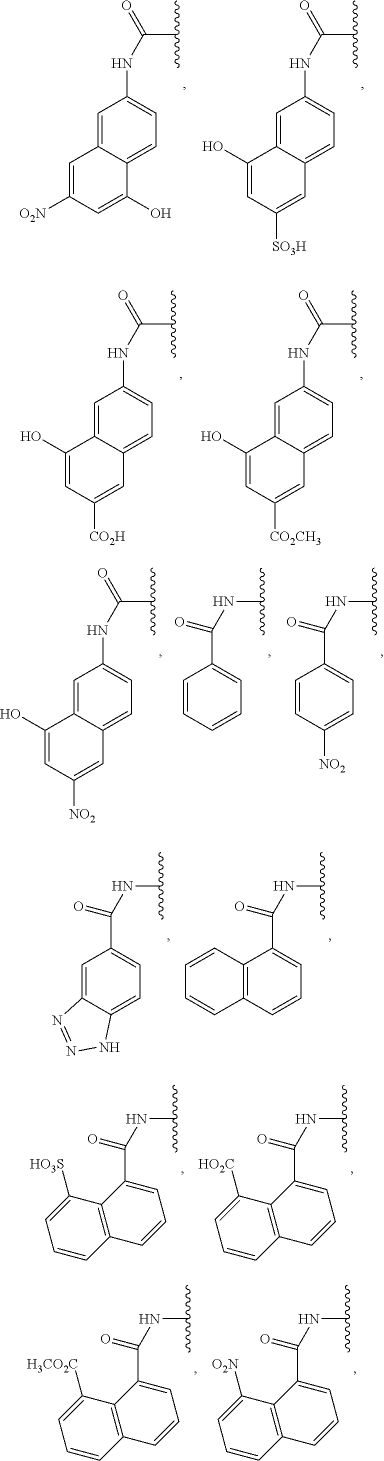

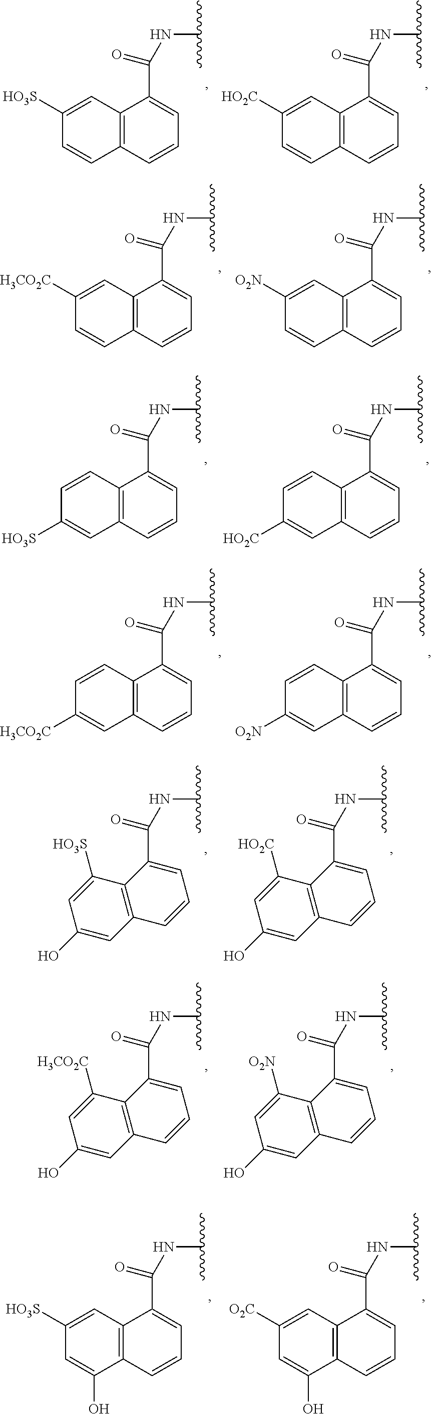

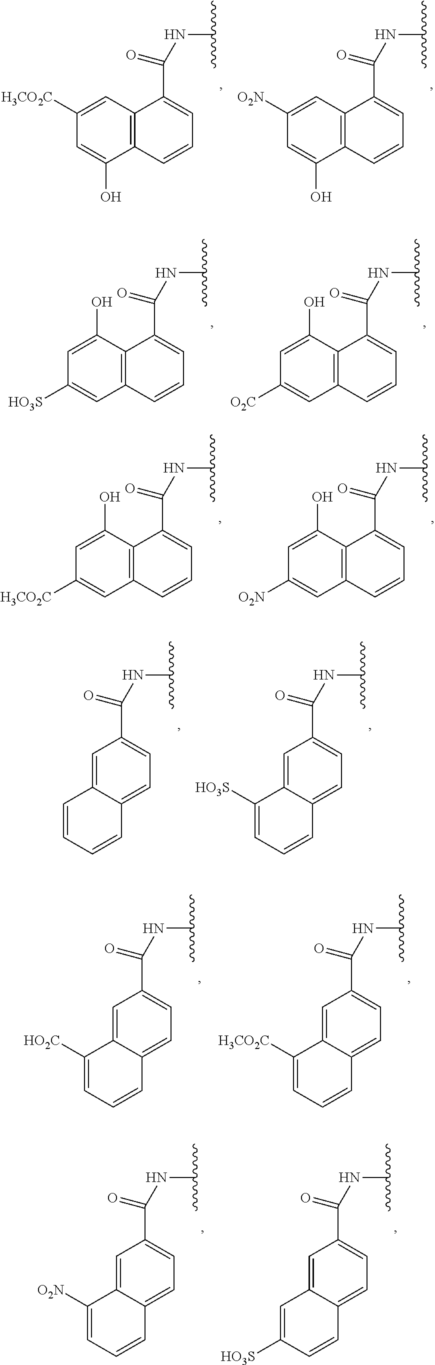

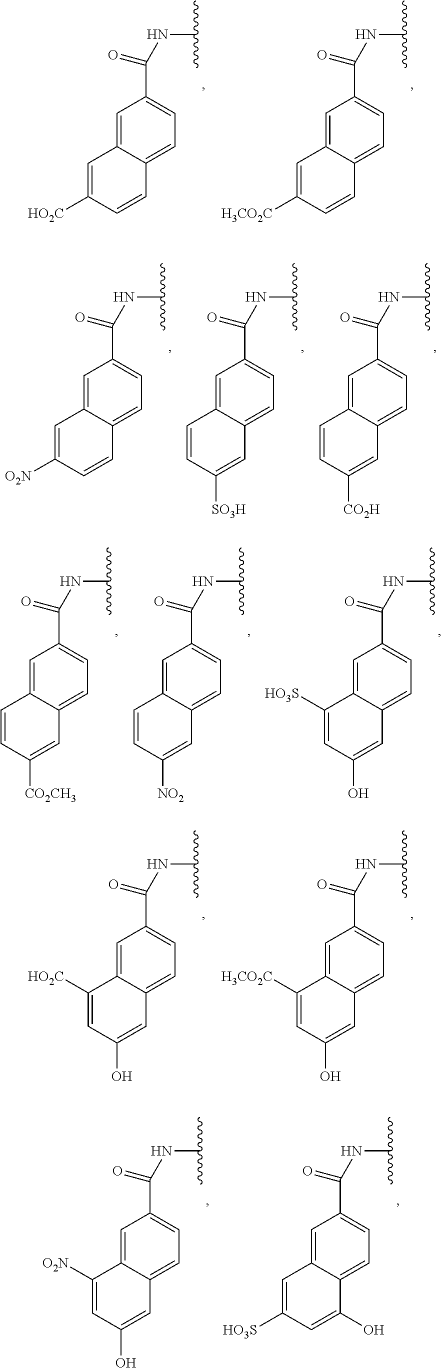

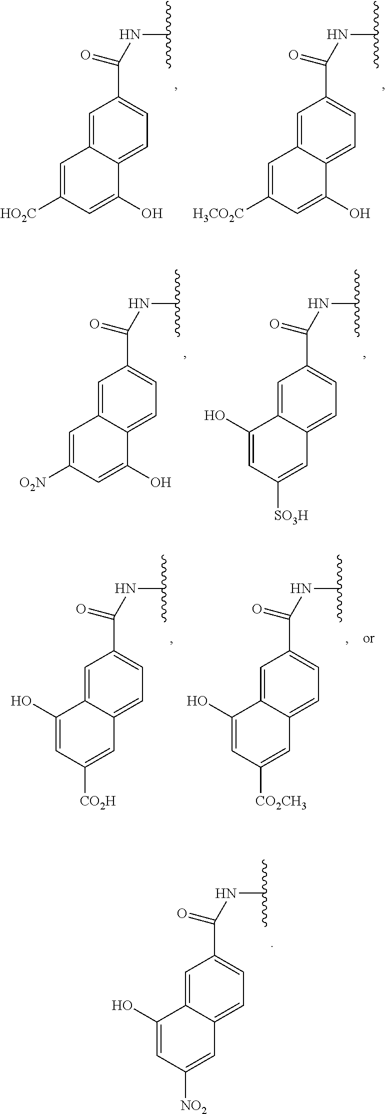

In embodiments, the compound has a structure of

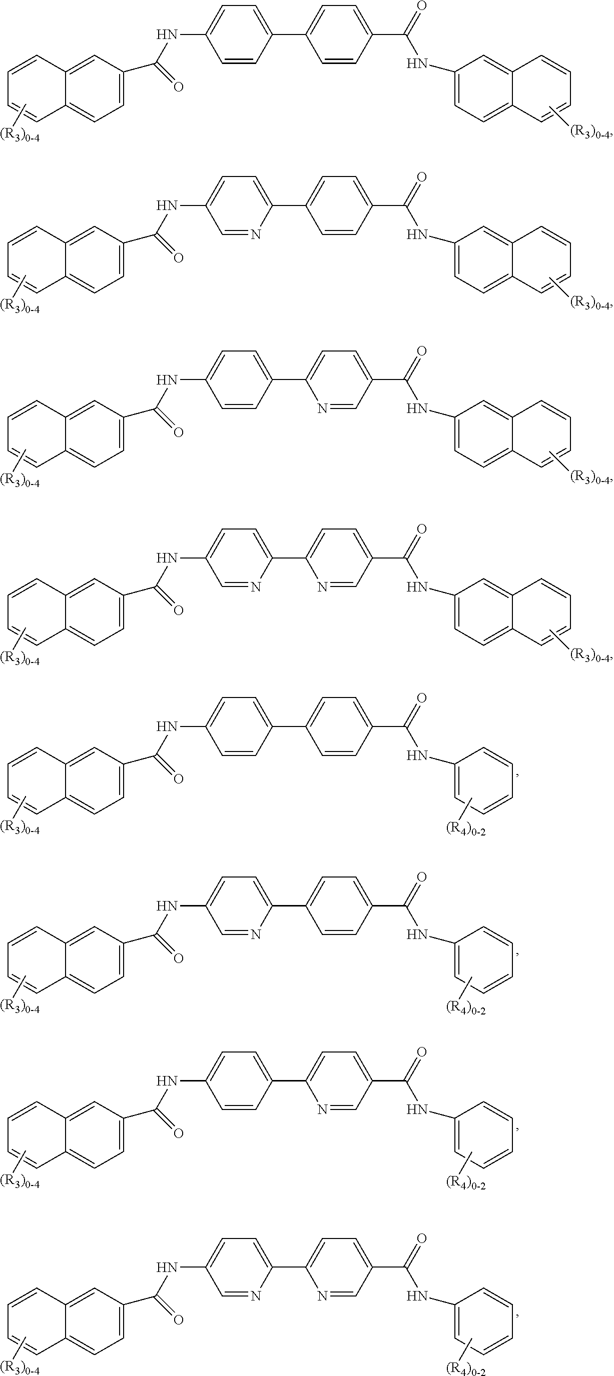

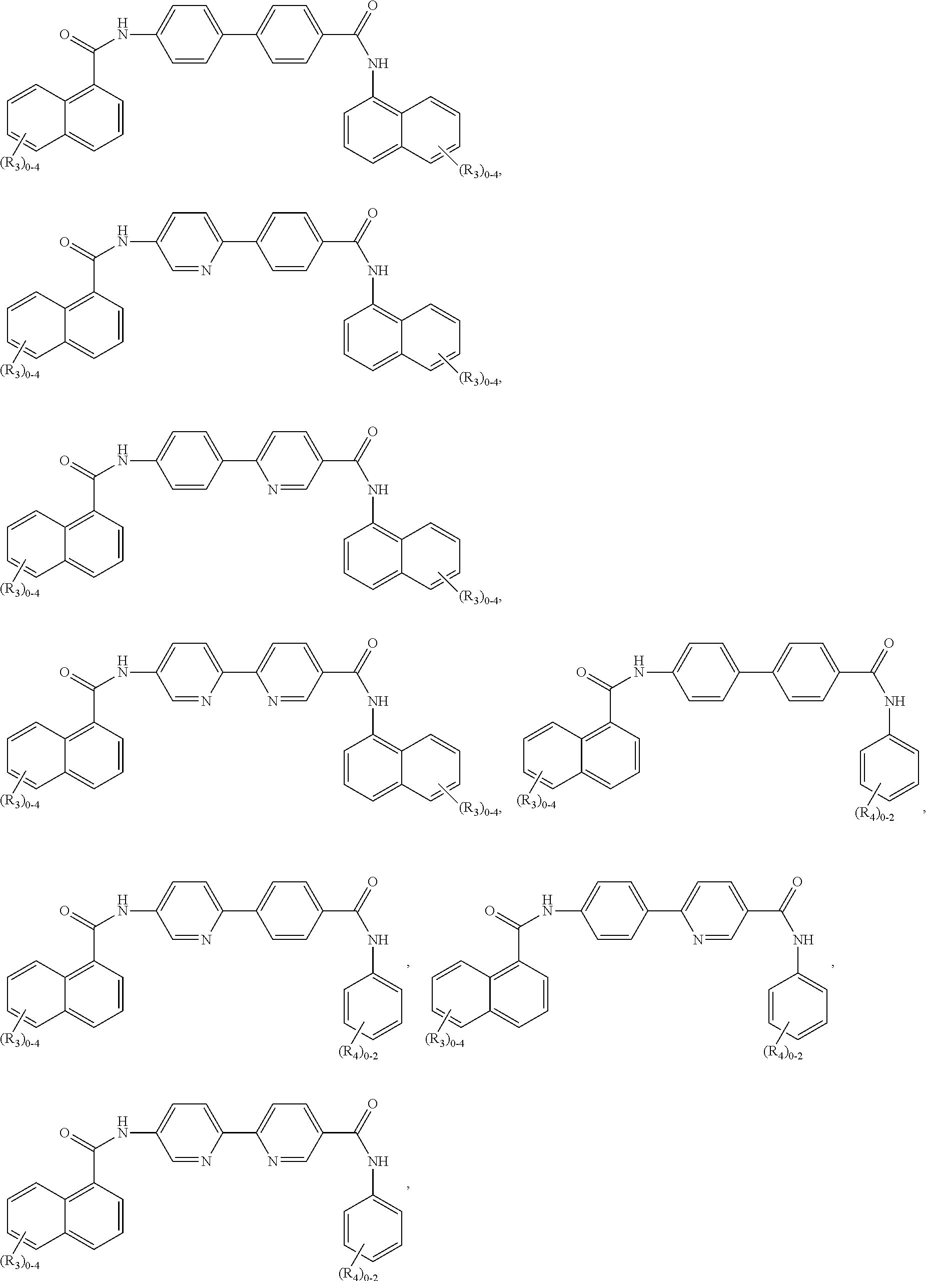

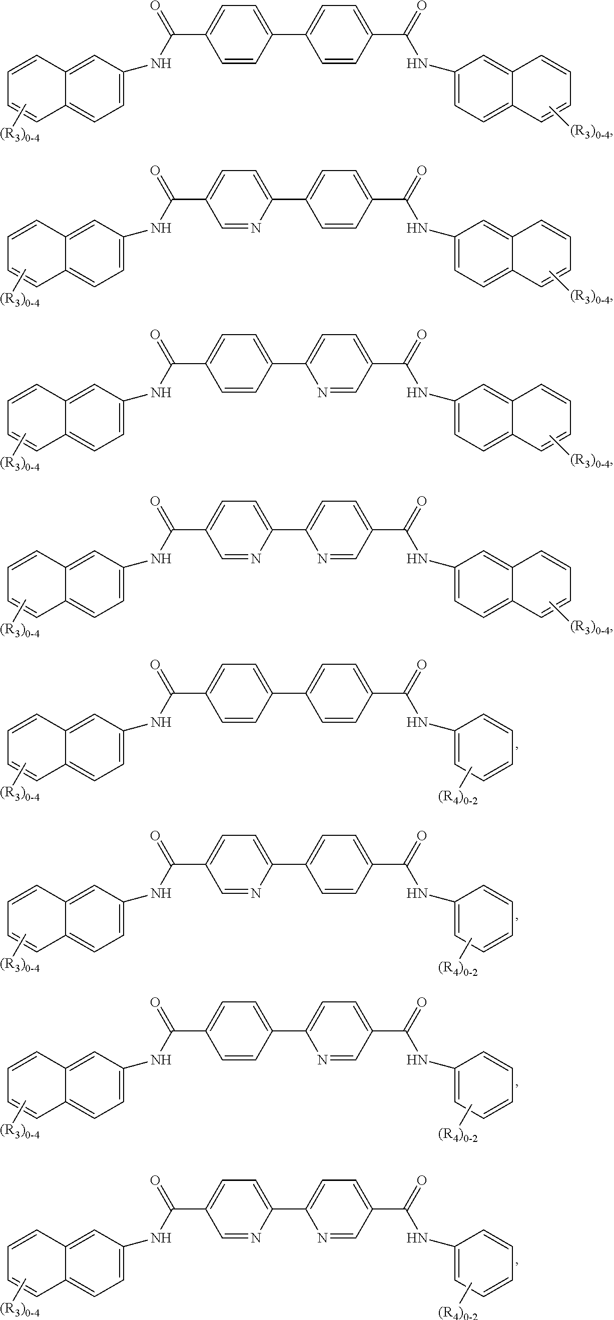

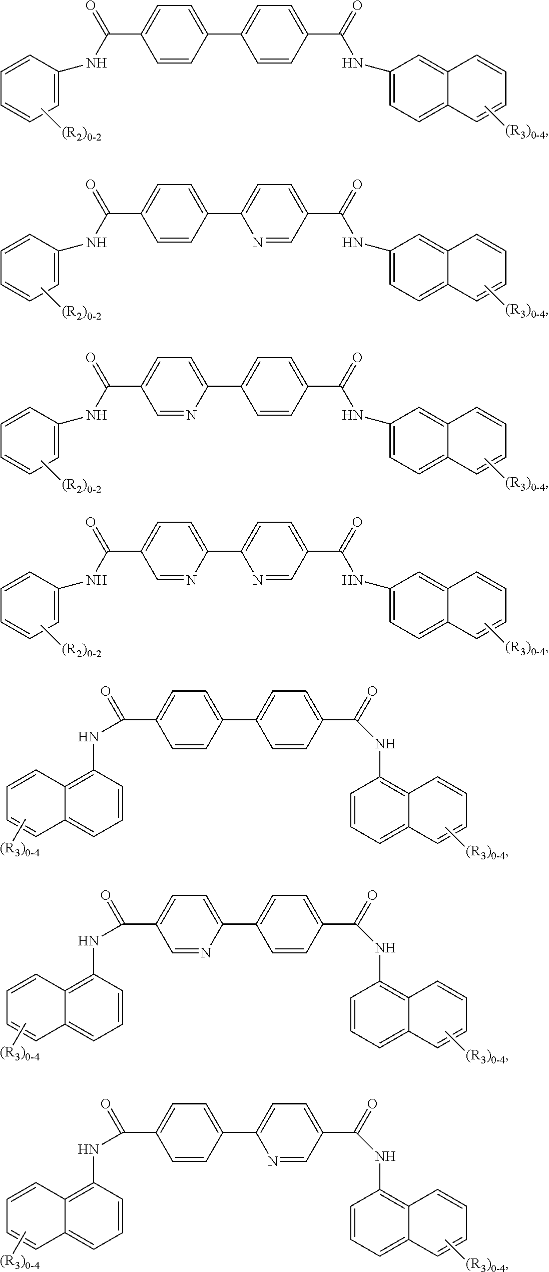

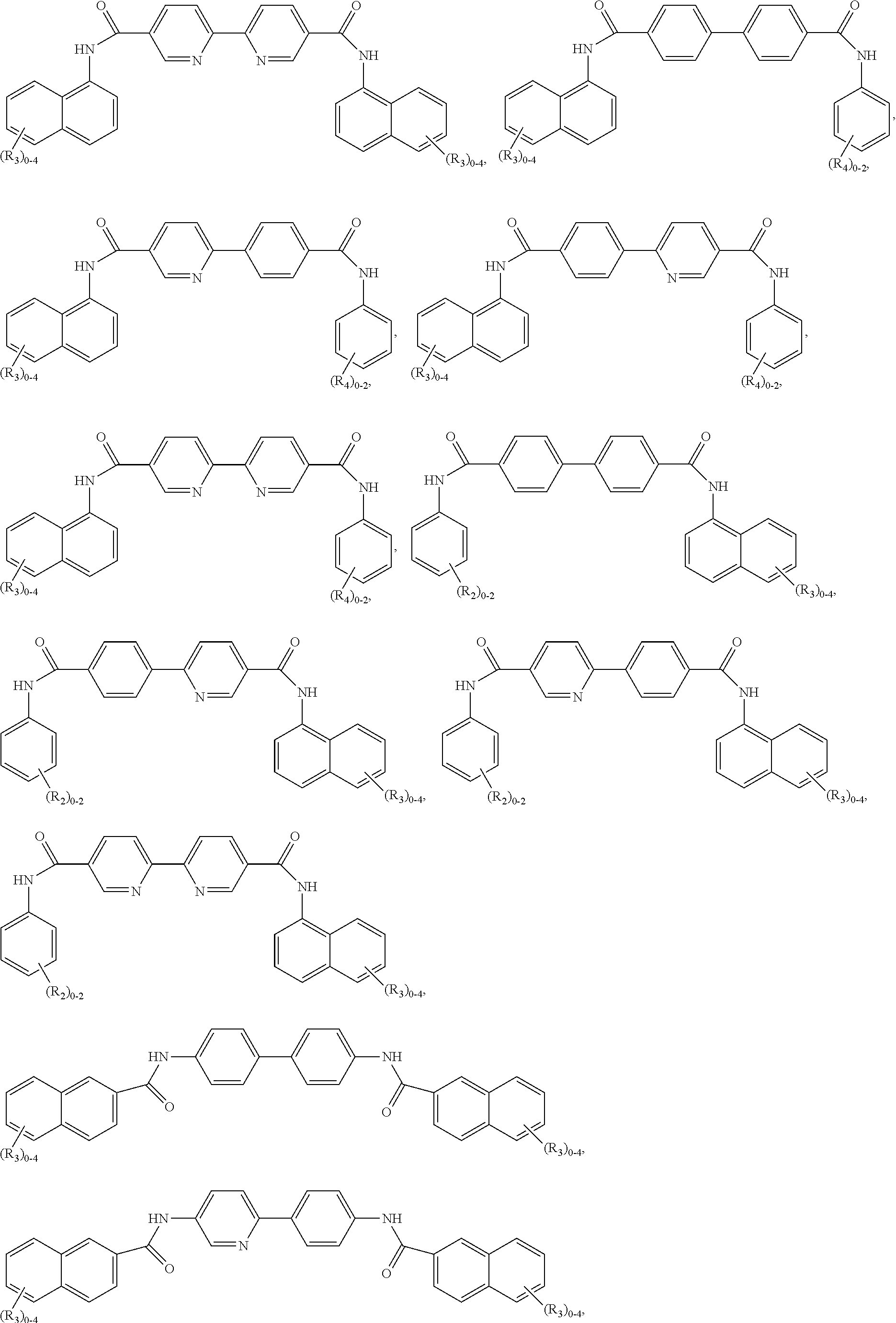

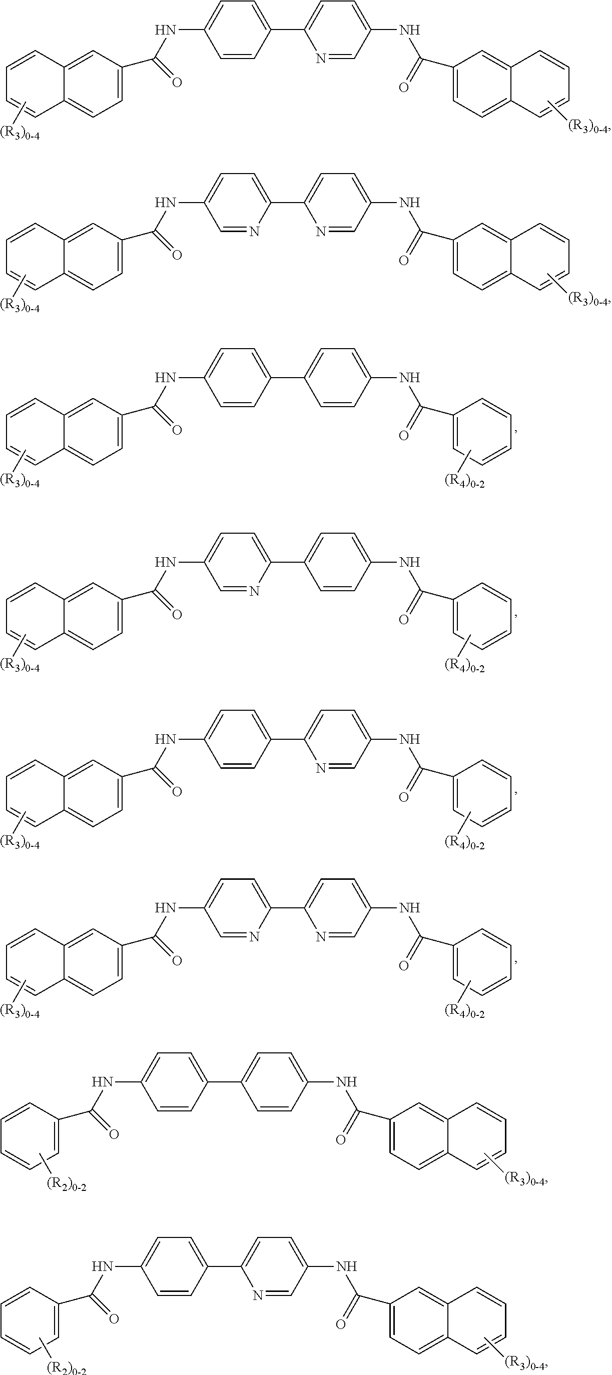

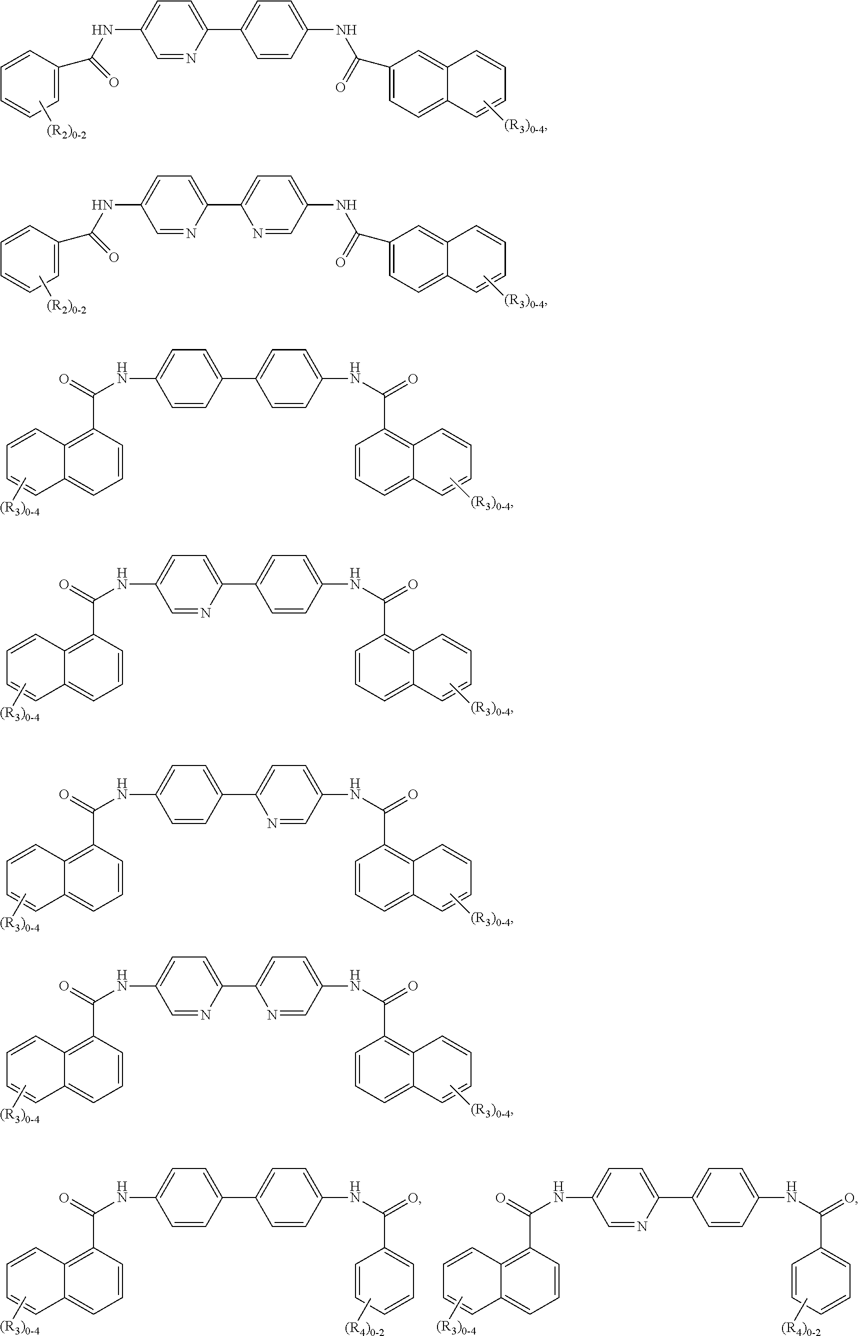

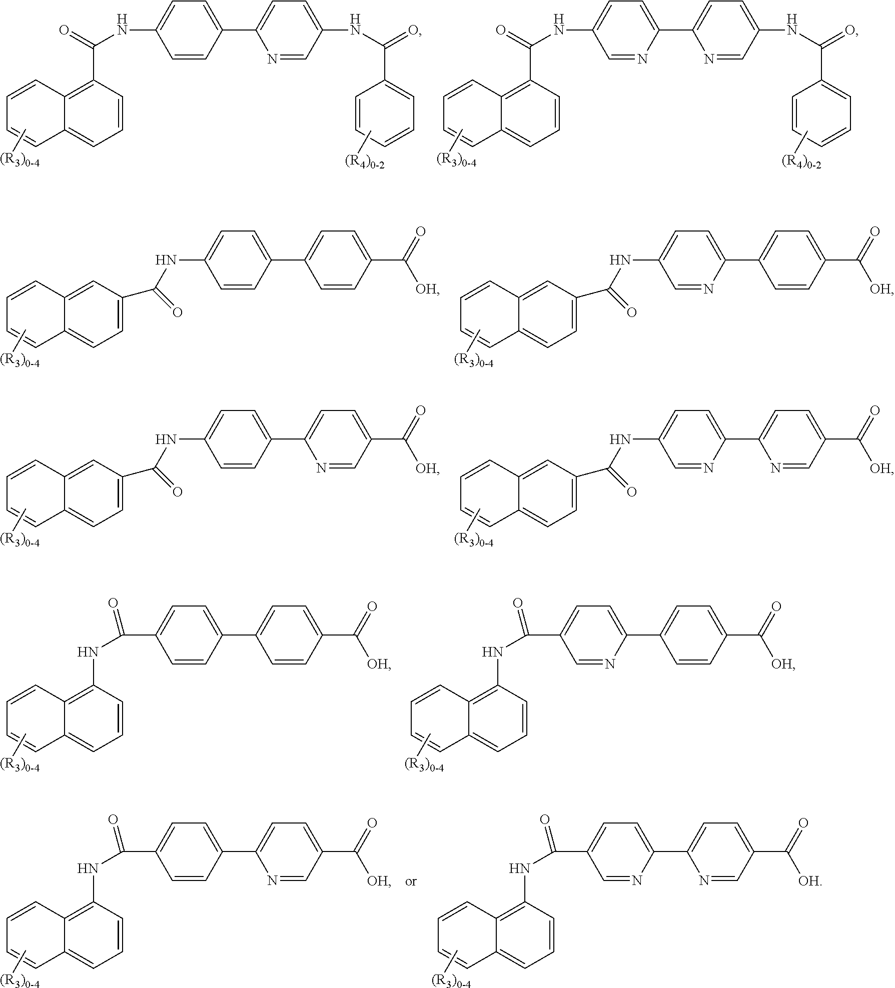

In embodiments, the compound has a structure of

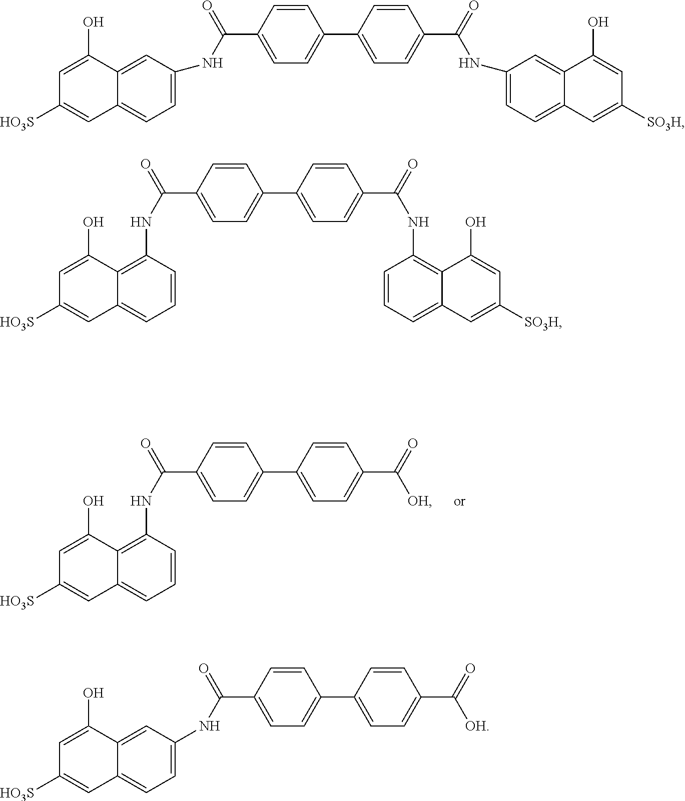

In embodiments, the compound has a structure of

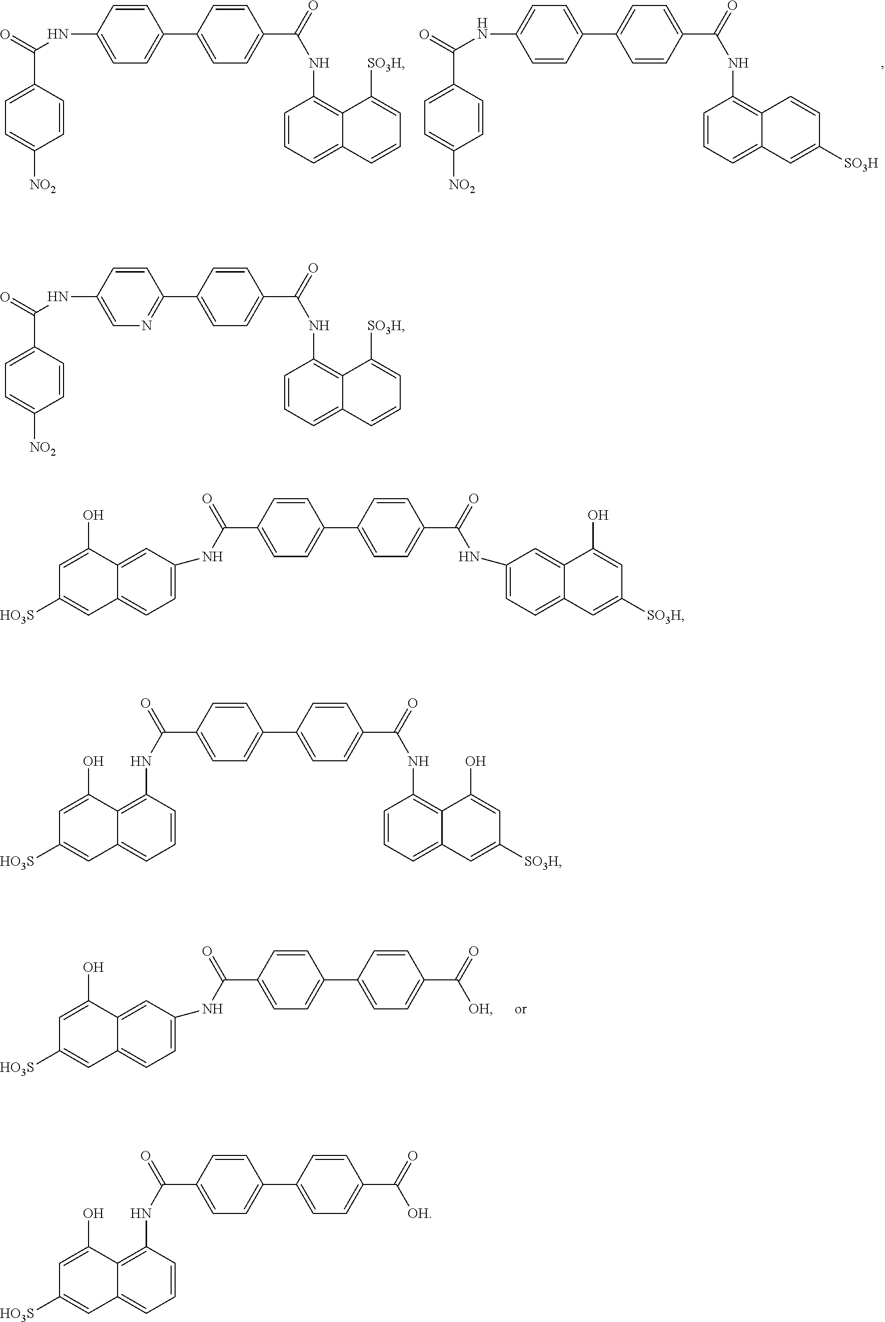

The compounds of the disclosure, or pharmaceutically acceptable salts thereof, can be identified as listed in Table A, below.

TABLE A

| | |
|---|---|
| A1 | 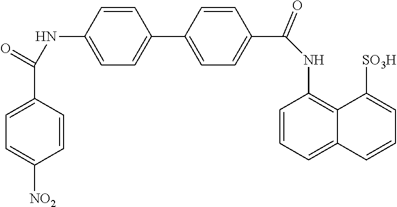 [SMILES: O=C(Nc1ccc(-c2ccc(C(=O)Nc3cccc4cccc(S(=O)(=O)O)c34)cc2)cc1)c1ccc([N+](=O)[O-])cc1] |
| A2 | 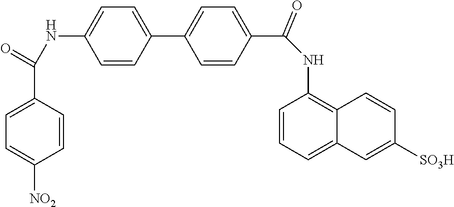 [SMILES: O=C(Nc1ccc(-c2ccc(C(=O)Nc3cccc4cc(S(=O)(=O)O)ccc34)cc2)cc1)c1ccc([N+](=O)[O-])cc1] |
| A3 | 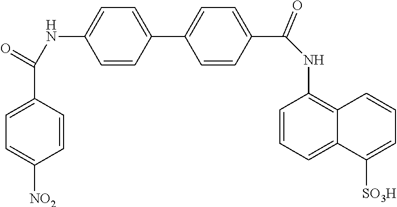 [SMILES: O=C(Nc1ccc(-c2ccc(C(=O)Nc3cccc4c(S(=O)(=O)O)cccc34)cc2)cc1)c1ccc([N+](=O)[O-])cc1] |
| A4 | 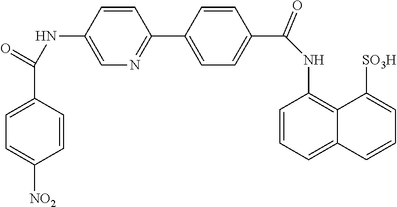 [SMILES: O=C(Nc1ccc(-c2ccc(C(=O)Nc3cccc4cccc(S(=O)(=O)O)c34)cc2)nc1)c1ccc([N+](=O)[O-])cc1] |
| A5 | 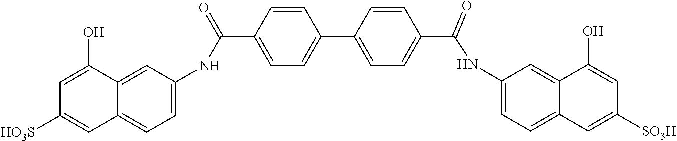 [SMILES: O=C(Nc1ccc2cc(S(=O)(=O)O)cc(O)c2c1)c1ccc(-c2ccc(C(=O)Nc3ccc4cc(S(=O)(=O)O)cc(O)c4c3)cc2)cc1] |
| A6 | 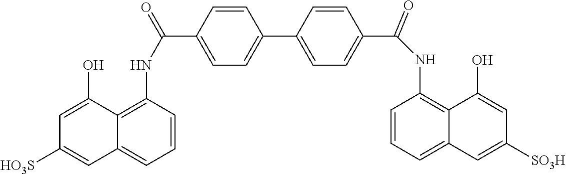 [SMILES: O=C(Nc1cccc2cc(S(=O)(=O)O)cc(O)c12)c1ccc(-c2ccc(C(=O)Nc3cccc4cc(S(=O)(=O)O)cc(O)c34)cc2)cc1] |
| A7 | 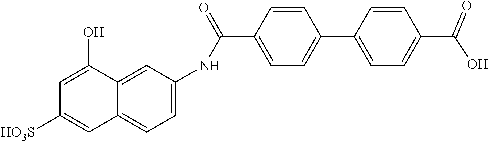 [SMILES: O=C(O)c1ccc(-c2ccc(C(=O)Nc3ccc4cc(S(=O)(=O)O)cc(O)c4c3)cc2)cc1] |
| A8 | 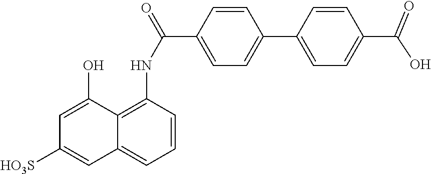 [SMILES: O=C(O)c1ccc(-c2ccc(C(=O)Nc3cccc4cc(S(=O)(=O)O)cc(O)c34)cc2)cc1] |

The compounds described herein can be functionalized further using techniques that are generally known in the art. For example, carbonyl-containing compounds can be further manipulated using all classical carbonyl functionalization strategies, such as alkylation, addition, reduction, olefination, etc., as well as combinations thereof.

The compounds disclosed herein can be present as a salt. Salts of the compounds disclosed herein include those derived from suitable inorganic or organic acids or bases. Examples of acid addition salts are salts formed with inorganic acids such as hydrochloric acid, hydrobromic acid, phosphoric acid, sulfuric acid and perchloric acid or with organic acids such as acetic acid, trifluoroacetic acid, oxalic acid, maleic acid, tartaric acid, citric acid, succinic acid or malonic acid or by using other methods used in the art such as ion exchange. Salts can also be prepared by reacting with a suitable base. Such salts include, but are not limited to, alkali metal, alkaline earth metal, aluminum salts, ammonium, N*(C₁₋₄alkyl)₄ salts, and salts of organic bases such as trimethylamine, triethylamine, morpholine, pyridine, piperidine, picoline, dicyclohexylamine, N,N-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N-bisdehydroabietylamine, glucamine, N-methylglucamine, collidine, quinine, quinoline, and basic amino acids such as lysine and arginine. The salt can also be formed from the quaternization of any basic nitrogen-containing groups of the compounds disclosed herein. Representative alkali or alkaline earth metal salts include sodium, lithium, potassium, calcium, magnesium, and the like. Some acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, glutamate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Further salts include ammonium, quaternary ammonium, and amine cations formed using counterions such as halide, hydroxide, carboxylate, sulfate, phosphate, nitrate, lower alkyl sulfonate and aryl sulfonate.

### Pharmaceutical Compositions

The disclosure further provides pharmaceutical compositions comprising the compounds or salts described herein and a pharmaceutically acceptable carrier. The carrier can include an excipient.

The phrase “pharmaceutically acceptable” is employed herein to refer to those ligands, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Compositions described herein can be administered in various forms, depending on the disorder to be treated and the age, condition, and body weight of the patient, as is well known in the art. For example, where the compositions are to be administered orally, they may be formulated as tablets, capsules, granules, powders, or suspensions; or for parenteral administration, they may be formulated as injections (intravenous, intramuscular, or subcutaneous), drop infusion preparations, or suppositories. In embodiments, the compositions can be administered via inhalation or via intranasal administration. These compositions can be prepared by conventional means in conjunction with the methods described herein, and, if desired, the active ingredient may be mixed with any conventional additive or excipient, such as a binder, a disintegrating agent, a lubricant, a corrigent, a solubilizing agent, a suspension aid, an emulsifying agent, or a coating agent. In embodiments, the composition is an oral composition. In some cases, the oral composition is a tablet, capsule, or suspension.

The phrase “pharmaceutically acceptable carrier” as used herein means a pharmaceutically acceptable material, composition, or vehicle, such as a liquid or solid filler, diluent, excipient, solvent or encapsulating material. As used herein the language “pharmaceutically acceptable carrier” includes buffers, sterile water for injection, solvents, dispersion media, coatings, antibacterial and antifungal agents, isotonic and absorption delaying agents, and the like, compatible with pharmaceutical administration. Each carrier must be “acceptable” in the sense of being compatible with the other ingredients of the composition and not injurious to the patient. Some examples of materials which can serve as pharmaceutically acceptable carriers include: (1) sugars, such as lactose, glucose, and sucrose; (2) starches, such as corn starch, potato starch, and substituted or unsubstituted cyclodextrins (α-, β-, or γ-cyclodextrins); (3) cellulose, and its derivatives, such as sodium carboxymethyl cellulose, ethyl cellulose, cellulose acetate, hydroxypropyl methylcellulose (HPMC), hydroxypropyl methylcellulose acetate succinate (HPMCAS); (4) polymers such as polyvinylpyrrolidone (PVP), polyvinylpyrrolidone-vinyl acetate (PVP/VA); (5) surfactants such as sodium lauryl sulfate, polysorbates (Tween), polyoxyethylene stearates (Myri), polyoxyethylene alkyl ethers (Brij), polyethylene glycol, polyvinyl acetate and polyvinylcaprolactame-based graft copolymer (Soluplus), D-α-tocopheryl polyethylene glycol 1000 succinate (TPGS); (6) gelatin; (7) talc; (8) excipients, such as cocoa butter and suppository waxes; (9) lipids such as Captex, Capmul and Cremophore; (10) oils, such as peanut oil, cottonseed oil, safflower oil, sesame oil, olive oil, corn oil, and soybean oil; (11) glycols, such as propylene glycol; (12) polyols, such as glycerin, sorbitol, mannitol, and polyethylene glycol; (13) esters, such as ethyl oleate and ethyl laurate; (14) buffering agents, such as magnesium hydroxide and aluminum hydroxide; (15) alginic acid; (16) pyrogen-free water; (17) isotonic saline; (18) Ringers solution; (19) ethyl alcohol; (20) phosphate buffer solutions; and (21) other non-toxic compatible substances employed in pharmaceutical compositions. In certain embodiments, pharmaceutical compositions provided herein are non-pyrogenic, i.e., do not induce significant temperature elevations when administered to a patient.

Wetting agents, drug solubilizers, dispersing agents, emulsifiers such as sodium lauryl sulfate, Tween, Brij, Myri, Solubplus, and lubricants such as magnesium stearate, as well as coloring agents, release agents, coating agents, sweetening, flavoring, and perfuming agents, preservatives and antioxidants can also be present in the compositions as excipients. Prevention of the action of microorganisms may be ensured by the inclusion of various antibacterial and antifungal agents, for example, paraben, chlorobutanol, phenol sorbic acid, and the like. It may also be desirable to include tonicity-adjusting agents, such as sugars and the like into the compositions. In addition, prolonged absorption of an injectable pharmaceutical form may be brought about by the inclusion of agents which delay absorption such as aluminum monostearate and gelatin.

Precipitation inhibitors or compound stabilizers that prevent or slow compound conversions during processing, storage, and dissolution, such as surfactants, lipids, complexing agents, and polymers can also be present in the composition.

Examples of pharmaceutically acceptable antioxidants as excipient include: (1) water soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The pharmaceutical compositions can be included in a container, pack, or dispenser together with instructions for administration.

Actual dosage levels of the active ingredients in the pharmaceutical compositions provided herein may be varied so as to obtain “therapeutically effective amount,” which is an amount of the active ingredient effective to achieve the desired therapeutic response for a particular patient, composition, and mode of administration, without being toxic to the patient.

The concentration of a compound provided herein in a pharmaceutically acceptable mixture will vary depending on several factors, including the dosage of the compound to be administered, the pharmacokinetic characteristics of the compound employed, and the route of administration. Typical dose ranges can include from about 0.01 to about 50 mg/kg of body weight per day, given in 1-4 divided doses. The dosage will be a therapeutically effective amount depending on several factors including the overall health of a patient, and the composition and route of administration of the compound.

### Method and Uses of the Small Molecule Inhibitors

Also provided herein are uses and methods of preventing or treating a viral infection in a subject using the compounds, salts, or pharmaceutical compositions of the disclosure.

In particular, the disclosure provides uses of the compounds, salts, or pharmaceutical compositions as a medicament in a mammal, for example, a human. In embodiments, the medicament treats a viral infection. In embodiments, the viral infection is caused by a coronavirus.

There are several possible targets in the coronavirus life cycle for therapeutic interventions, for example, attachment and entry, uncoating, gRNA replication, translation in endoplasmic reticulum (ER) and Golgi, assembly, and virion release. Of these targets, viral attachment and entry are advantageous, as they are the first steps in the replication cycle or the virus and take place at a relatively accessible extracellular site. In general, coronaviruses (CoVs) use their glycosylated S protein to bind to their cognate cell surface receptors and initiate membrane fusion and virus entry. For example, for each of SARS-CoV and SARS-CoV-2 (i.e., the latter of which is a cause of COVID-19), the spike protein S mediates entry into cells by binding to angiotensin converting enzyme 2 (ACE2) via its receptor-binding domain (RBD) followed by proteolytic activation by human proteases. Thus, blockade of this RBD-ACE2 protein-protein interaction (PPI) can disrupt infection efficiency. The compounds and pharmaceutical compositions of the disclosure can block this interaction. While CoVs can utilize different receptors for binding, several CoVs, even from different genera, can utilize the same receptor. For example, SARS-CoV-2 is the third human CoV utilizing ACE2 as its cell entry receptor, the other two being SARS-CoV and α-coronavirus HCoV NL63. MERS-CoV can use dipeptidyl peptidase 4 (DPP4) for entry, while HCoV 229E can utilize CD13 for entry. Some p-coronaviruses (e.g., HCoV OC43) can bind to sialic acid receptors.

As described below in the examples and in Bojadzic et al., ACS Infect. Dis. 2021, 7, 6, 1519-1534, herein incorporated by reference in its entirety, the Compounds A1, A2, A3, A4, and A5 can successfully inhibit the attachment and entry of a coronavirus to a host cell. Without intending to be bound by theory, it is believed that the polar substituent-containing aromatic ring framework of these compounds interact with the trimeric S-protein of the CoV to inhibit the PPI of these S proteins with their host cell receptor needed for viral attachment. Accordingly, because all of the compounds of the disclosure have a structure including the same framework as Compounds A1-A5, the compounds of disclosure are expected to have the same function, i.e., interacting with the S-protein and inhibiting the PPI needed for viral attachment.

In embodiments, the coronavirus is SARS-CoV, SARS-CoV-2, HCoV-NL63, MERS-CoV, HCoV-229E, HCoV-OC43, HCoV-HKU1, or a combination thereof. In embodiments, the coronavirus is SARS-CoV. In embodiments, the coronavirus is SARS-CoV-2. In embodiments, the coronavirus is HCoV-NL63. In embodiments, the coronavirus is MERS-CoV. In embodiments, the coronavirus is HCoV-229E. In embodiments, the coronavirus is HCoV-OC43.

In embodiments, the coronavirus is HCoV-HKU1. In embodiments, use of the compounds or pharmaceutical compositions inhibit an interaction between a coronavirus spike protein and a receptor thereof, thereby decreasing viral attachment and entry into a host cell. In embodiments, the receptor is angiotensin converting enzyme 2 (ACE2), dipeptidyl peptidase 4 (DPP4), CD13, or a sialic acid receptor. In embodiments, the receptor is ACE2, DPP4, or CD13. In embodiments, the receptor is ACE2. In embodiments, the receptor is DPP4. In embodiments, the receptor is CD13.

Also provided are methods of preventing or treating a viral infection in a subject in need thereof, the method comprising administering to the subject a therapeutically effective amount of a compound of Formula (I). The compound can be administered as a pharmaceutical composition, as described herein. In embodiments, the viral infection is caused by a coronavirus as described herein.

It is understood that while the disclosure is read in conjunction with the detailed description thereof, the foregoing description and following examples are intended to illustrate and not limit the scope of the disclosure, which is defined by the scope of the appended claims. Other aspects, advantages, and modifications are within the scope of the following claims.

### EXAMPLES

### Example 1: Chemical Synthesis of Compounds

Synthesis of the compounds generally involved two amide couplings and one hydrogenation, as illustrated in Scheme 1, below. Different linkers and naphthyl moieties were used as needed for other structures.

All starting chemicals and reagents were obtained from SigmaAldrich (St. Louis, MO, USA) or VWR (Radnor, PA, USA). The compounds were characterized with 1H-NMR, 13C-NMR, high-resolution mass spectrometry (HRMS), and infrared (IR) spectroscopy. Mass spectra were obtained at the Mass Spectrometry Laboratory, Department of Chemistry, University of Florida (Gainesville, FL, USA). Low-resolution ES (electron spray) mass spectra were carried out with Finnigan LCQ DECA/Agilent 1100 LC/MS mass spectrometer (Thermo Fisher Scientific, Waltham, MA). High-resolution mass spectra were recorded on an Agilent 6220 ESI TOF (Santa Clara, CA, USA) mass spectrometer. Analysis of sample purity was performed on an Agilent (Palo Alto, CA, USA) 1100 series HPLC system with a ThermoScientific Hypurity C8 (5 μm; 2.1×100 mm+guard column). HPLC conditions were as follows: solvent A=water with 2 mM ammonium acetate, solvent B=methanol with 2 mM ammonium acetate, and flow rate=0.2 mL/min. Compounds were eluted with a gradient of A/B=80:20 at 0 min to 0:100 at 50 min. Purity was determined via integration of UV spectra at 254 nm, and all tested compounds have a purity of ≥95%.

### Synthesis of Compound A2

Compound A2 was prepared according to the reaction scheme provided in Scheme 1, below.

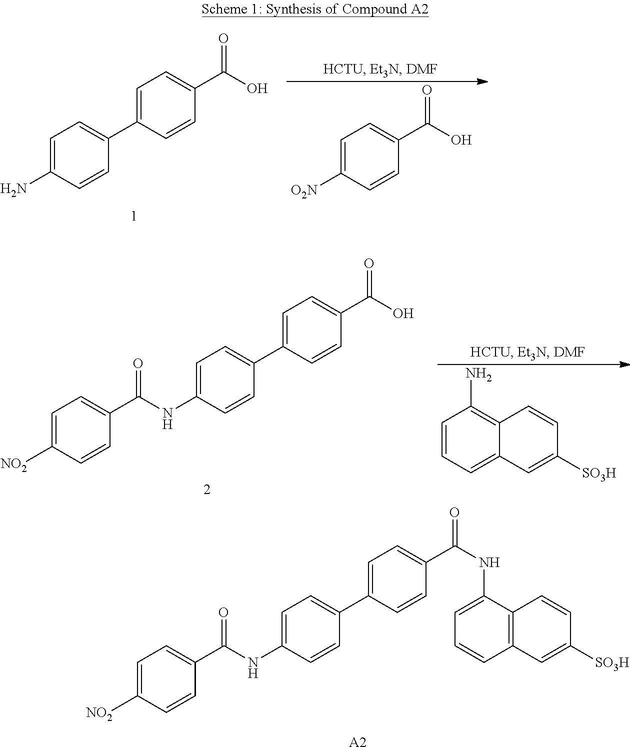

### Step 1: Synthesis of 4′-(4-Nitrobenzamido)biphenyl-4-carboxylic acid (Intermediate Compound 2)

The general procedure for coupling reaction as previously described was followed with 4-nitrobenzoic acid (compound 1; 2.8 g, 16.6 mmol) and 4′-aminobiphenyl-4-carboxylic acid (2.4 g, 11.3 mmol) to give the intermediate compound 2. Briefly, for the coupling reaction, under an argon atmosphere, trimethylamine (Et₃N) was added dropwise to a mixture of 4-nitrobenzoic acid, O-(6-chlorobenzotriazol-1-yl)-N,N,N′,N′-tetramethyluronium hexafluorophosphate (HCTU), and DMF at 0° C., and the resulting reaction mixture was stirred for 1 h at the same temperature, to give intermediate compound 2.

¹H NMR (500 MHz, DMSO-d6): δ 12.94 (br, 1H), 10.70 (s, 1H), 8.39 (d, J=8.6 Hz, 2H), 8.22 (d, J=8.7 Hz, 2H), 8.02 (d, J=8.2 Hz, 2H), 7.93 (d, J=8.5 Hz, 2H), 7.82 (d, J=8.2 Hz, 2H), 7.80 (d, J=8.5 Hz, 2H); ¹³C NMR (125 MHz, DMSO-d6): δ 167.1, 164.0, 149.2, 143.7, 140.5, 139.0, 134.5, 130.0, 129.27, 129.26, 127.3, 126.3, 123.6, 120.8; FTIR (neat) vmax 3301, 1694, 1632, 1593, 1569, 1515, 1419, 1391, 1347, 1330, 1301, 1255, 1199, 1107, 1005, 870, 856, 826, 798, 773, 712, 701, 672 cm⁻¹; HRMS (ESI) [M+H]⁺ calculated for C₂₀H₁₅N₂O₅*, 363.1; found, 363.1.

### Step 2: Synthesis of 5-(4′-(4-Nitrobenzamido)biphenyl-4-ylcarboxamido)naphthalene-2-sulfonic acid (Compound A2)

Intermediate compound 2 (181 mg, 0.5 mmol) was reacted with 5-aminonaphthalene-2-sulfonic acid (112 mg, 0.5 mmol) to give the triethylamine salt of Compound A2 as a yellowish solid (85 mg, 30%) (>99% pure by HPLC analysis (UV spectra at 254 nm)).

¹H NMR (500 MHz, DMSO-d6): δ 10.73 (s, 1H), 10.49 (s, 1H), 8.86 (br, 1H), 8.39 (d, J=8.4 Hz, 2H), 8.30-8.15 (m, 5H), 8.05-7.88 (m, 6H), 7.84 (d, J=8.4 Hz, 2H), 7.77 (d, J=8.8 Hz, 1H), 7.65 (d, J=7.1 Hz, 1H), 7.58 (t, J=7.9 Hz, 1H), 3.08 (q, J=6.8 Hz, 6H), 1.16 (t, J=7.2 Hz, 9H); ¹³C NMR (125 MHz, DMSO-d6): δ 165.8, 164.0, 149.2, 145.6, 142.6, 140.5, 138.9, 134.7, 133.8, 133.0, 132.9, 129.3, 128.9, 128.6, 127.2, 126.9, 126.2, 126.0, 124.4, 123.9, 123.6, 123.1, 120.9, 45.8, 8.6; HRMS (ESI) [M−H]⁻ calculated for C₃₀H₂₀N₃O₇S⁻, 566.1027; found, 566.1052.

### Synthesis of Compound A3

Compound A3 was prepared according to a reaction scheme similar to that provided in Scheme 1 only using a differently substituted aminonaphthalene-sulfonic acid in Step 2.

### Step 2: Synthesis of 5-(4′-(4-Nitrobenzamido)biphenyl-4-ylcarboxamido)naphthalene-1-sulfonic acid (Compound A3)

Intermediate compound 2 (181 mg, 0.5 mmol) was reacted with 5-aminonaphthalene-1-sulfonic acid (112 mg, 0.5 mmol) to give the triethylamine salt of Compound A3 as a yellowish solid (210 mg, 63%) (>99% pure by HPLC analysis (UV spectra at 254 nm)).

¹H NMR (500 MHz, DMSO-d₆): δ 10.73 (s, 1H), 10.49 (s, 1H), 8.86 (d, J=7.9 Hz, 2H), 8.39 (d, J=8.2 Hz, 2H), 8.24 (d, J=8.4 Hz, 2H), 8.21 (d, J=8.0 Hz, 2H), 8.02 (d, J=7.6 Hz, 2H), 7.97 (d, J=8.3 Hz, 2H), 7.90 (d, J=8.0 Hz, 2H), 7.85 (d, J=8.3 Hz, 2H), 7.59 (d, J=7.1 Hz, 1H), 7.56 (d, J=7.2 Hz, 1H), 7.49 (t, J=7.9 Hz, 1H), 3.07 (q, J=7.3 Hz, 6H), 1.16 (t, J=7.4 Hz, 9H); ¹³C NMR (125 MHz, DMSO-d₆): δ 165.8, 164.0, 149.2, 144.3, 142.5, 140.5, 138.8, 134.6, 133.7, 132.9, 130.0, 129.8, 129.3, 128.5, 127.2, 126.3, 126.2, 125.0, 124.8, 124.53, 124.46, 123.9, 123.6, 120.8, 45.8, 8.6; HRMS (ESI) [M−H]⁻ calcd. for C₃₀H₂₀N₃O₇S⁻, 566.1027; found, 566.1025.

### Scheme 1: Synthesis of Compound A5

Compound A5 was prepared according to the reaction scheme provided in Scheme 2, below.

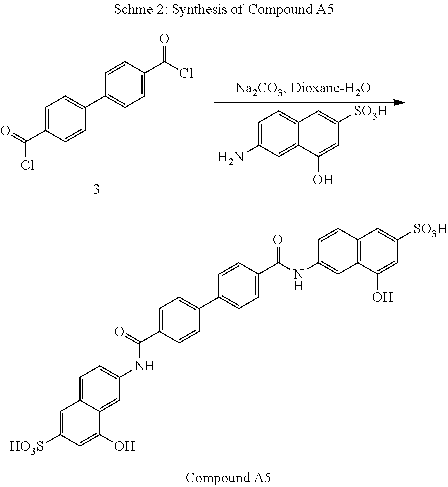 [SMILES: O=C(Cl)c1ccc(-c2ccc(C(=O)Cl)cc2)cc1 ; Nc1ccc2cc(S(=O)(=O)O)cc(O)c2c1 ; O=C(Nc1ccc2cc(S(=O)(=O)O)cc(O)c2c1)c1ccc(-c2ccc(C(=O)Nc3ccc4cc(S(=O)(=O)O)cc(O)c4c3)cc2)cc1]

Compound 3, 4,4′-biphenyldicarbonyl chloride (76.7 mg, 0.275 mmol) was added to a solution of 6-amino-4-hydroxynaphthalene-2-sulfonic acid (120 mg, 0.5 mmol) in dioxane (2 mL) and water (2 mL) by portion at room temperature. During addition of 3, the pH value was kept within 4.0 to 5.0 by adding 1 N sodium carbonate dropwise. After reaction, the pH value was adjusted to 2. Dioxane and water were removed by high vacuum pump. The residue was transferred to a test tube and taken up with methanol at 80° C. 2.0 mL of water was added. The reaction mixture was cooled to room temperature and filtered to give Compound A5 as a red solid (234 mg, 100%) (>99% pure by HPLC analysis (UV spectra at 254 nm)).

¹H NMR (500 MHz, DMSO-d₆): δ 10.49 (s, 2H), 10.11 (s, 2H), 8.63 (d, J=2.0 Hz, 2H), 8.17 (d, J=8.3 Hz, 4H), 7.98 (d, J=8.3 Hz, 4H), 7.91 (dd, J=8.9, 2.1 Hz, 2H), 7.85 (d, J=8.9 Hz, 2H), 7.56 (s, 2H), 7.15 (d, J=1.5 Hz, 2H); ¹³C NMR (100 MHz, DMSO-d₆): δ 164.8, 163.0, 152.1, 150.5, 150.3, 145.0, 141.8, 136.0, 134.1, 130.7, 130.6, 128.3, 126.7, 124.5, 121.0; HRMS (ESI) [M−2H]²⁻ calcd. for C₃₄H₂₂N₂O₁₀S₂²⁻, 682.68; found 683.08.

### Example 2: Binding of CoV S Proteins to Human ACE2

The feasibility of establishing a screening assay using a cell-free ELISA-type 96-well format with Fc-conjugated receptors coated on the plate and Flag- or His-tagged ligands in the solution was evaluated. To establish assay conditions, concentration-response assessments using such a format with ACE2-Fc and SARS-CoV-2 S1 or RBD with His tag were performed.

ACE2-Fc and SARS-CoV-2 S1 or RBD with His tag proteins used in the binding assay were obtained from SinoBiological (Wayne, PA, USA); catalog no. 10108-H05H, 40592-V08H, and 40591-V08H). SARS-CoV S1S2 and HCov NL63 S1 were from the same provider (SinoBiological; catalog no. 40634-V08B and 40600-V08H). Binding inhibition assays were performed in a 96-well cell-free format.

Microtiter plates (Nunc F Maxisorp, 96-well; Thermo Fisher Scientific, Waltham, MA, USA) were coated overnight at 4° C. with 100 μL/well of Fc-conjugated ACE2 receptor diluted in PBS pH 7.2. This was followed by blocking with 200 μL/well of SuperBlock (PBS) (Thermo Fisher Scientific) for 1 hour at RT (about 23° C.). Then, plates were washed twice using washing solution (PBS pH 7.4, 0.05% Tween-20) and tapped dry before the addition of the tagged ligand (SARS-CoV-2 S1 or RBD) and test compounds diluted in binding buffer (100 mM HEPES, pH 7.2) to give a total volume of 100 μL/well. After 1 h incubation, three washes were conducted, and a further 1 h incubation with anti-His HRP conjugate (BioLegend; San Diego, CA, USA; catalog no. 652504) diluted (1:2500) in SuperBlock (PBS) was used to detect the bound His-tagged ligand. Plates were washed four times before the addition of 100 μL/well of HRP substrate TMB (3,3′,5,5′-tetramethylbenzidine) and kept in the dark for up to 15 min. The reaction was stopped using 20 μL of 1M H₂SO₄, and the absorbance value was read at 450 nm. The plated concentrations of ACE2 receptor were 1.0 μg/mL for SARS-CoV-2 RBD and 2.0 μg/mL for SARS-CoV-2 S1. The concentrations of the ligand used in the inhibitory assays were 0.5 μg/mL for RBD and 1.0 μg/mL for S1.

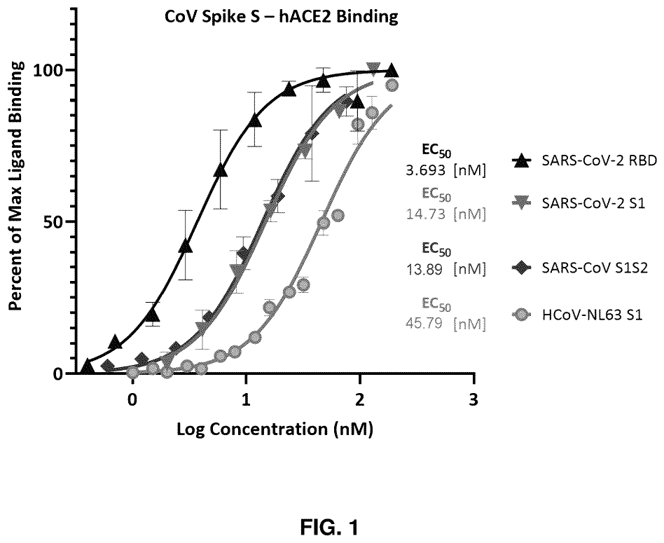

These assays indicated that both bindings follow classic sigmoid patterns with a slightly stronger binding for RBD than S1 (FIG. 1). Fitting of the data gave median effective concentrations (EC₅₀s) and binding affinity constant (Kd) estimates of 4 and 15 nM, respectively (98 and 1125 ng/mL). These values were in good agreement with published values that are also in the low nanomolar range (4-90 nM), typically based on surface plasmon resonance (SPR) studies.

Binding was also quantified for SARS-CoV S1S2 and HCoV-NL62, which are two other coronaviruses that also bind to the ACE2. The results indicated a slightly weaker binding for SARS-CoV S1S2 (14 nM) and a much weaker binding for the common cold causing coronavirus HCov-NL63 (46 nM) (FIG. 1).

### Example 3: Cell-Free Screening Assay

An inhibitory screening with hACE2 and SARS-CoV-2 RBD-His, as it showed stronger binding, was performed. Concentrations of 1 and 0.5 μg/mL for ACE2 and SARS-CoV-2-RBD, respectively, were selected to produce high-enough signals and performed a preliminary screening of representative compounds from an in-house library of about 100 compounds, including the various compounds of the disclosure. Additional compounds screened included chloroquine, clemastine, and suramin, as each has been considered of possible interest in inhibiting SAR-CoV-2 by different mechanisms of action.

Screening at 5 μM indicated that many of the tested compounds had no activity and, hence, were unlikely to interfere with the S-protein-ACE2 binding needed for viral attachment. However, several compounds showed promising inhibitory activity (e.g., >60% inhibition at 5 μM). The compounds showing this inhibitory activity were selected for further evaluation.

### Example 4: Cell-Free Binding Inhibition Assays

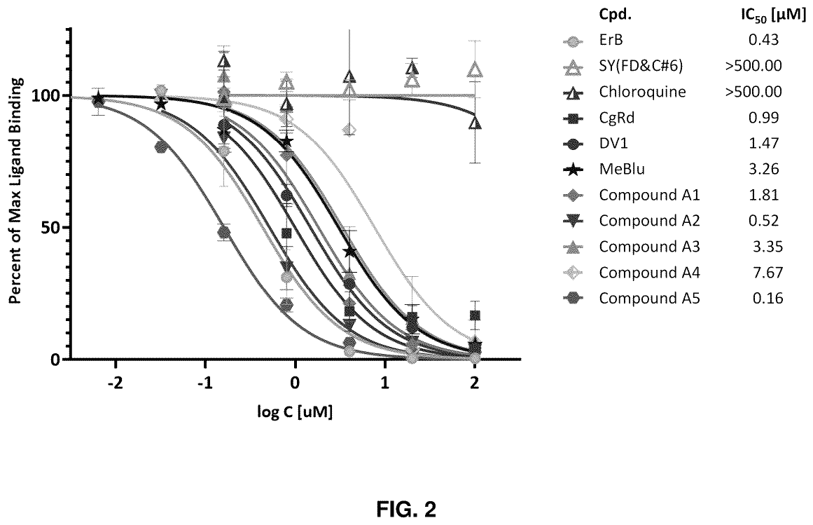

To confirm the hits indicated by the screening assay, detailed concentration-response assessments with selected compounds were performed. As shown in FIG. 2, several of the tested compounds had low micromolar or even sub-micromolar inhibitory activity in the SARS-CoV-2 S-ACE2 assay.

For example, among tested organic dyes, Congo red (CgRd), direct violet 1 (DV1), Evans blue (EvBI), chlorazol black (ChBI), calcomine scarlet 3B (CSc3B), and methylene blue (MeBI) had IC₅₀s of 0.99, 1.47, 2.25, 2.57, 4.25, and 3.26 μM, respectively. Further, Compounds A1-A5 also had good inhibitory activity with Compound A5 and Compound A2 having the best IC₅₀s of 160 and 520 nM (FIG. 2).

To ensure that inhibition is not due to polymolecular conglomeration or aggregation, which is often the cause of promiscuous inhibition with pan-assay interference compounds (PAINS), a nonionic detergent (Triton-X 100, 0.01%) was also added to the binding inhibitory assay as recommended for the detection of such effects. Addition of Triton caused no significant deterioration in the inhibitory effects on SARS-CoV-2 RBD binding for the compounds tested; for example, for Compound A2 IC₅₀s changed from 0.52 μM (95% Cl: 0.42-0.63) to 0.85 μM (95% Cl: 0.62-1.18).

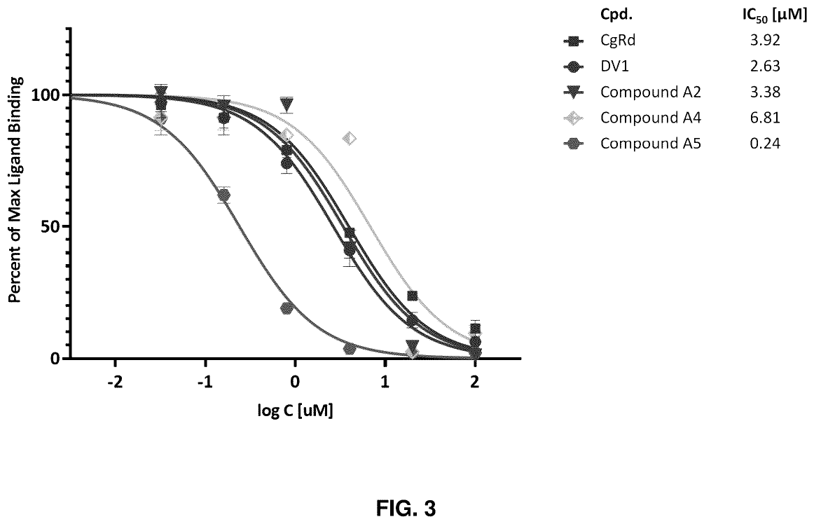

The ability of the compounds of the disclosure to inhibit the corresponding interaction of the S protein of SARS-CoV was also assessed, using its S1S2 portion; SinoBiological; catalog no. 40634-V08B; ACE2-Fc: 2 μg/mL, His-tagged S1S2: 1.0 μg/mL, i.e., a SARS-CoV S-hACE2 assay. As shown in FIG. 3, several of the same compounds including organic dyes (CgRd, DV1, and others) as well as compounds of the disclosure (e.g., Compounds A2 and A5) showed similar activity against SARS-CoV as against SARS-CoV-2.

For compounds tested in this assay such as Congo red (CgRd), direct violet 1 (DV1), Evans blue (EvBI), calcomine scarlet 3B (CSc3B), Compound A2, and Compound A5, the IC₅₀s were 3.9, 2.6, 1.3, 9.9, 3.4, and 0.24 μM, respectively. These results demonstrated that broad-spectrum activity can be achieved with the compounds of the disclosure, which is unlikely with most antibodies.

### Example 5: Protein Thermal Shift for Assessment of Binding Partner

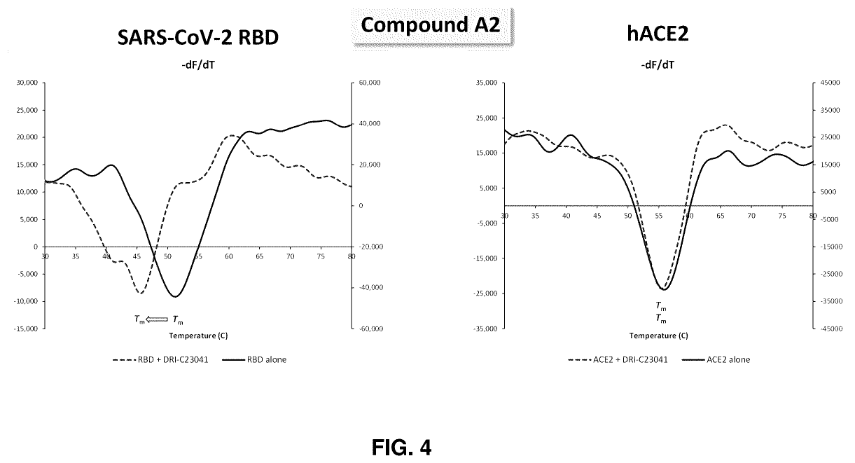

As an additional binding assay and to establish whether the compounds of the disclosure invention bind to CoV-S or ACE2, a protein thermal shift (differential scanning fluorimetry or ThermoFluor) assay was used. This assay quantifies the shift in protein stability caused by binding of a ligand via use of a dye whose fluorescence increases when exposed to hydrophobic surfaces, which happens as the protein starts to unfold as it is heated and exposes its normally buried hydrophobic core residues. It allows rapid and inexpensive evaluations of the temperature-dependence of protein stability using real-time PCR instruments and only small amounts of protein. The presence of Compound A2 caused a clear left-shift in the melting temperature (Tm) of the protein for SARS-CoV-2-RBD, but not ACE2 (dashed vs continuous line) indicating the former as the binding partner (FIG. 4).

### Example 6: Pseudovirus Assay (BacMam, HEK293 Cells)

Inhibitory activity on pseudovirus entry using pseudoviruses bearing the SARS-CoV-2 S spike protein (plus fluorescent reporters) generated using BacMam-based tools (Montana Molecular, Bozeman, MT; cat. No. C1100G) was confirmed. These pseudoviruses do not require high containment level (biosafety level 3, BSL-3), as they do not replicate in human cells, and allow quantification of viral entry as they express bright green fluorescent protein that are targeted to the nucleus of the ACE2- and red fluorescence expressing host cell (here, HEK293 cells from ATCC, Manassas, VA, USA; cat. no. CRL-1573). Generally, one day after entry, the host cell expresses green fluorescent protein in the nucleus indicating pseudovirus entry. If viral entry is blocked, the cell nucleus will be dark.

For this assay, fluorescent biosensors from Montana Molecular (Bozeman, MT, USA) were used per the instructions of the manufacturer with minor modifications. Briefly, HEK293T cells (ATCC, Manassas, VA, USA) were seeded onto 96-well plates at a density of 50,000 cells per well in 100 μL complete medium (DMEM supplemented with 10% fetal bovine serum). A transduction mixture containing ACE2 BacMan Red-Reporter virus (1.8×10⁸ Vg/mL) and 2 mM sodium butyrate prepared in complete medium was added (50 μL per well) and incubated for 24 h at 37° C. and 5% CO₂. Medium was removed, washed once with PBS, and replaced with 100 μL fresh medium containing the compound under study, pre-incubating for 30 min at 37° C. and 5% CO₂. A transduction mixture containing Pseudo SARS-CoV-2 Green-Reporter pseudovirus (3.3×10⁸ Vg/mL) and 2 mM sodium butyrate prepared in complete medium was added (50 μL per well) and incubated for 48 h at 37° C. and 5% CO₂. The medium was removed, washed once with PBS, replaced with 150 μL fresh medium, and cells incubated for additional 48 h at 37° C. and 5% CO₂. Cell fluorescence was detected using an EVOS FL microscope (Life Technologies, Carlsbad, CA, USA) and quantified using the Analyze Particles tool after thresholding for the corresponding colors in ImageJ (US National Institutes of Health, Bethesda, MD, USA).

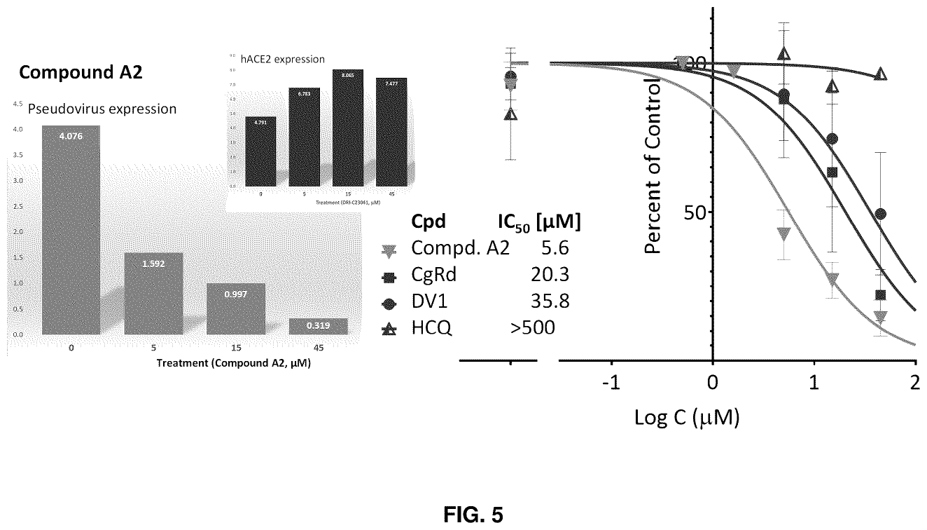

The fluorescence associated with ACE2 and pseudovirus entry were quantified through the corresponding areas of the fluorescence images. A representative set of quantification is shown in the plot on the left side of FIG. 5 while the composite concentration-response curves are on the right. Compound A2 showed excellent concentration-dependent inhibition, as quantified by the amount of green area assessed over fully confluent sections. In particular, while CgRd had a micromolar IC₅₀ value of 20 μM, Compound A2 performed even better, with an IC₅₀ values of 5.6 μM, as shown in FIG. 5.

### Example 7: Pseudovirus Assay (VSV-ΔG, Vero-E6 Cells)

A second confirmatory assay has been done with a different pseudovirus (SARS-CoV-2 spike plus GFP reporter bearing VSV-ΔG pseudovirus, i.e., vesicular stomatitis virus that lacks the VSV envelope glycoprotein) and cell line (ACE2/Furin-overexpressing Vero-E6 cells). Vero-E6 cells (African Green Monkey renal epithelial cells; ATCC cat. no. CRL-1586) engineered to overexpress hACE2/Furin were seeded in 24-well plates to obtain a confluence of 80%. The medium was replaced with 250 μL cell culture medium (DMEM) supplemented with 2% fetal bovine serum, 1% penicillin/streptomycin/glutamine, and the compounds of interest for 30 min. Cells were inoculated with the SARS-CoV-2 spike protein pseudotyped VSV-ΔG (multiplicity of infection=0.05) by adding complete media to bring the final volume to 400 μL, and 20 h post infection, plates were scanned with a 10× objective using the Incucyte ZOOM imaging system (Sartorius, Ann Arbor, MI, USA). Normalized GFP expression (GCU) values per image were obtained by dividing the Total Green Object Integrated Intensity [Green Calibrated Units (GCU)×μm²/image] values of each image by its corresponding Total Phase Area (μm²/image) as described before.

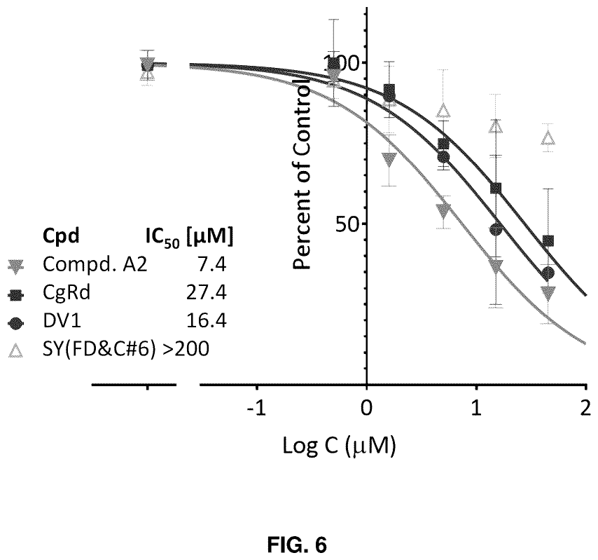

GFP fluorescence quantified this way was used as a measure of infection, and normalized values were fitted with regular concentration response curves as before. Obtained inhibitory effects (FIG. 6) were very consistent with those from the previous assay with IC₅₀'s of 7 and 16 μM for Compound A2 and CgRd, respectively, confirming the antiviral potential of these compounds.

### REFERENCES

1. Liu, P. P.; Blet, A.; Smyth, D.; Li, H. The science underlying COVID-19: implications for the cardiovascular system. Circulation 2020, 142, 68-78.
2. Moore, J. B.; June, C. H. Cytokine release syndrome in severe COVID-19. Science 2020, 368, 473-474.
3. Matheson, N. J.; Lehner, P. J. How does SARS-CoV-2 cause COVID-19? Science 2020, 369, 510-511.
4. Shang, J.; Wan, Y.; Luo, C.; Ye, G.; Geng, Q.; Auerbach, A.; Li, F. Cell entry mechanisms of SARS-CoV-2. Proc. Natl. Acad. Sci. U.S.A. 2020, 117, 11727-11734.
5. Lan, J.; Ge, J.; Yu, J.; Shan, S.; Zhou, H.; Fan, S.; Zhang, Q.; Shi, X.; Wang, Q.; Zhang, L.; Wang, X. Structure of the SARS-CoV-2 spike receptor-binding domain bound to the ACE2 receptor. Nature 2020, 581, 215-220.
6. Sivaraman, H.; Er, S. Y.; Choong, Y. K.; Gavor, E.; Sivaraman, J. Structural basis of the SARS-CoV-2/SARS-CoV receptor binding and small-molecule blockers as potential therapeutics. Annu. Rev. Pharmacol. Toxicol. 2021, 61, 465-493.
7. Guy, R. K.; DiPaola, R. S.; Romanelli, F.; Dutch, R. E. Rapid repurposing of drugs for COVID-19. Science 2020, 368, 829-830.
8. Tiwari, V.; Beer, J. C.; Sankaranarayanan, N. V.; Swanson-Mungerson, M.; Desai, U. R. Discovering small-molecule therapeutics against SARS-CoV-2. Drug Discov. Today 2020, 25, 1535-1544.
9. Melby, T.; Westby, M. Inhibitors of viral entry. Handb. Exp. Pharmacol. 2009, 177-202.
10. Sui, J.; Deming, M.; Rockx, B.; Liddington, R. C.; Zhu, Q. K.; Baric, R. S.; Marasco, W. A. Effects of human anti-spike protein receptor binding domain antibodies on severe acute respiratory syndrome coronavirus neutralization escape and fitness. J. Virol. 2014, 88, 13769-13780.
11. Wang, C.; Li, W.; Drabek, D.; Okba, N. M. A.; van Haperen, R.; Osterhaus, A.; van Kuppeveld, F. J. M.; Haagmans, B. L.; Grosveld, F.; Bosch, B. J. A human monoclonal antibody blocking SARS-CoV-2 infection. Nat. Commun. 2020, 11, 2251.
12. Wu, Y.; Wang, F.; Shen, C.; Peng, W.; Li, D.; Zhao, C.; Li, Z.; Li, S.; Bi, Y.; Yang, Y.; Gong, Y.; Xiao, H.; Fan, Z.; Tan, S.; Wu, G.; Tan, W.; Lu, X.; Fan, C.; Wang, Q.; Liu, Y.; Zhang, C.; Qi, J.; Gao, G. F.; Gao, F.; Liu, L. A noncompeting pair of human neutralizing antibodies block COVID-19 virus binding to its receptor ACE2. Science 2020, 368, 1274-1278.
13. Arkin, M. R.; Wells, J. A. Small-molecule inhibitors of protein-protein interactions: progressing towards the dream. Nat. Rev. Drug Discov. 2004, 3, 301-317.
14. Buchwald, P. Small-molecule protein-protein interaction inhibitors: therapeutic potential in light of molecular size, chemical space, and ligand binding efficiency considerations. IUBMB Life 2010, 62, 724-731.
15. Scott, D. E.; Bayly, A. R.; Abell, C.; Skidmore, J. Small molecules, big targets: drug discovery faces the protein-protein interaction challenge. Nat. Rev. Drug Discov. 2016, 15, 533-550.
16. Huck, B. R.; Kötzner, L.; Urbahns, K. Small molecules drive big improvements in immuno-oncology therapies. Angew. Chem. Int. Ed. Engl. 2018, 57, 4412-4428.
17. Bojadzic, D.; Buchwald, P. Toward small-molecule inhibition of protein-protein interactions: General aspects and recent progress in targeting costimulatory and coinhibitory (immune checkpoint) interactions. Curr. Top. Med. Chem. 2018, 18, 674-699.
18. Margolles-Clark, E.; Umland, O.; Kenyon, N. S.; Ricordi, C.; Buchwald, P. Small molecule costimulatory blockade: organic dye inhibitors of the CD40-CD154 interaction. J. Mol. Med. 2009, 87, 1133-1143.
19. Margolles-Clark, E.; Kenyon, N. S.; Ricordi, C.; Buchwald, P. Effective and specific inhibition of the CD40-CD154 costimulatory interaction by a naphthalenesulphonic acid derivative. Chem. Biol. Drug Des. 2010, 76, 305-313.
20. Song, Y.; Margolles-Clark, E.; Bayer, A.; Buchwald, P. Small-molecule modulators of the OX40-OX40L costimulatory protein-protein interaction. Br. J. Pharmacol. 2014, 171, 4955-4969.
21. Chen, J.; Song, Y.; Bojadzic, D.; Tamayo-Garcia, A.; Landin, A. M.; Blomberg, B. B.; Buchwald, P. Small-molecule inhibitors of the CD40-CD40L costimulatory protein-protein interaction. J. Med. Chem. 2017, 60, 8906-8922.
22. Bojadzic, D.; Chen, J.; Alcazar, O.; Buchwald, P. Design, synthesis, and evaluation of novel immunomodulatory small molecules targeting the CD40-CD154 costimulatory protein-protein interaction. Molecules 2018, 23, 1153.
23. Bojadzic, D.; Buchwald, P. The CD40-targeting KGYY15 peptides do not efficiently block the CD40-CD40L interaction. Diabetologia 2019, 62, 2158-2160.
24. Buchwald, P. Inhibitors of TNF superfamily costimulatory interactions and methods for uses of the same. U.S. Pat. No. 10,562,848 B2, 2020.
25. Schirmer, R. H.; Adler, H.; Pickhardt, M.; Mandelkow, E. “Lest we forget you—methylene blue . . . ” Neurobiol. Aging 2011, 32, 2325 e7-16.
26. Lo, J. C.; Darracq, M. A.; Clark, R. F. A review of methylene blue treatment for cardiovascular collapse. J. Emerg. Med. 2014, 46, 670-679.
27. Salje, H.; Tran Kiem, C.; Lefrancq, N.; Courtejoie, N.; Bosetti, P.; Paireau, J.; Andronico, A.; Hoze, N.; Richet, J.; Dubost, C. L.; Le Strat, Y.; Lessler, J.; Levy-Bruhl, D.; Fontanet, A.; Opatowski, L.; Boelle, P. Y.; Cauchemez, S. Estimating the burden of SARS-CoV-2 in France. Science 2020, 369, 208-211.
28. Tai, W.; He, L.; Zhang, X.; Pu, J.; Voronin, D.; Jiang, S.; Zhou, Y.; Du, L. Characterization of the receptor-binding domain (RBD) of 2019 novel coronavirus: implication for development of RBD protein as a viral attachment inhibitor and vaccine. Cell. Mol. Immunol. 2020, 17, 613-620.
29. Hofmann, H.; Simmons, G.; Rennekamp, A. J.; Chaipan, C.; Gramberg, T.; Heck, E.; Geier, M.; Wegele, A.; Marzi, A.; Bates, P.; Pohlmann, S. Highly conserved regions within the spike proteins of human coronaviruses 229E and NL63 determine recognition of their respective cellular receptors. J. Virol. 2006, 80, 8639-8652.
30. Tortorici, M. A.; Walls, A. C.; Lang, Y.; Wang, C.; Li, Z.; Koerhuis, D.; Boons, G. J.; Bosch, B. J.; Rey, F. A.; de Groot, R. J.; Veesler, D. Structural basis for human coronavirus attachment to sialic acid receptors. Nat. Struct. Mol. Biol. 2019, 26, 481-489.
31. Leader, B.; Baca, Q. J.; Golan, D. E. Protein therapeutics: a summary and pharmacological classification. Nat. Rev. Drug Discov. 2008, 7, 21-39.
32. Suntharalingam, G.; Perry, M. R.; Ward, S.; Brett, S. J.; Castello-Cortes, A.; Brunner, M. D.; Panoskaltsis, N. Cytokine storm in a phase 1 trial of the anti-CD28 monoclonal antibody TGN1412. N. Engl. J. Med. 2006, 355, 1018-1028.
33. Wadman, M. London® disastrous drug trial has serious side effects for research. Nature 2006, 440, 388-389.
34. Downing, N. S.; Shah, N. D.; Aminawung, J. A.; Pease, A. M.; Zeitoun, J. D.; Krumholz, H. M.; Ross, J. S. Postmarket safety events among novel therapeutics approved by the US Food and Drug Administration between 2001 and 2010. J. Am. Med. Assoc. (JAMA) 2017, 317, 1854-1863.
35. Wec, A. Z.; Wrapp, D.; Herbert, A. S.; Maurer, D. P.; Haslwanter, D.; Sakharkar, M.; Jangra, R. K.; Dieterle, M. E.; Lilov, A.; Huang, D.; Tse, L. V.; Johnson, N. V.; Hsieh, C. L.; Wang, N.; Nett, J. H.; Champney, E.; Burnina, I.; Brown, M.; Lin, S.; Sinclair, M.; Johnson, C.; Pudi, S.; Bortz, R., 3rd; Wirchnianski, A. S.; Laudermilch, E.; Florez, C.; Fels, J. M.; OBrien, C. M.; Graham, B. S.; Nemazee, D.; Burton, D. R.; Baric, R. S.; Voss, J. E.; Chandran, K.; Dye, J. M.; McLellan, J. S.; Walker, L. M. Broad neutralization of SARS-related viruses by human monoclonal antibodies. Science 2020, 369, 731-736.
36. Xiu, S.; Dick, A.; Ju, H.; Mirzaie, S.; Abdi, F.; Cocklin, S.; Zhan, P.; Liu, X. Inhibitors of SARS-CoV-2 entry: current and future opportunities. J. Med. Chem. 2020, 63, 12256-12274.
37. Ju, B.; Zhang, Q.; Ge, J.; Wang, R.; Sun, J.; Ge, X.; Yu, J.; Shan, S.; Zhou, B.; Song, S.; Tang, X.; Yu, J.; Lan, J.; Yuan, J.; Wang, H.; Zhao, J.; Zhang, S.; Wang, Y.; Shi, X.; Liu, L.; Zhao, J.; Wang, X.; Zhang, Z.; Zhang, L. Human neutralizing antibodies elicited by SARS-CoV-2 infection. Nature 2020, 584, 115-119.
38. Baum, A.; Fulton, B. O.; Wloga, E.; Copin, R.; Pascal, K. E.; Russo, V.; Giordano, S.; Lanza, K.; Negron, N.; Ni, M.; Wei, Y.; Atwal, G. S.; Murphy, A. J.; Stahl, N.; Yancopoulos, G. D.; Kyratsous, C. A. Antibody cocktail to SARS-CoV-2 spike protein prevents rapid mutational escape seen with individual antibodies. Science 2020, 369, 1014-1018.
39. Arkin, M. R.; Tang, Y.; Wells, J. A. Small-molecule inhibitors of protein-protein interactions: Progressing toward the reality. Chem. Biol. 2014, 21, 1102-1114.
40. Milroy, L. G.; Grossmann, T. N.; Hennig, S.; Brunsveld, L.; Ottmann, C. Modulators of protein-protein interactions. Chem. Rev. 2014, 114, 4695-4748.
41. Song, Y.; Buchwald, P. TNF superfamily protein-protein interactions: feasibility of small-molecule modulation. Curr. Drug. Targets 2015, 16, 393-408.
42. Mullard, A. 2015 FDA drug approvals. Nat. Rev. Drug Discov. 2016, 15, 73-76.
43. Souers, A. J.; Leverson, J. D.; Boghaert, E. R.; Ackler, S. L.; Catron, N. D.; Chen, J.; Dayton, B. D.; Ding, H.; Enschede, S. H.; Fairbrother, W. J.; Huang, D. C.; Hymowitz, S. G.; Jin, S.; Khaw, S. L.; Kovar, P. J.; Lam, L. T.; Lee, J.; Maecker, H. L.; Marsh, K. C.; Mason, K. D.; Mitten, M. J.; Nimmer, P. M.; Oleksijew, A.; Park, C. H.; Park, C. M.; Phillips, D. C.; Roberts, A. W.; Sampath, D.; Seymour, J. F.; Smith, M. L.; Sullivan, G. M.; Tahir, S. K.; Tse, C.; Wendt, M. D.; Xiao, Y.; Xue, J. C.; Zhang, H.; Humerickhouse, R. A.; Rosenberg, S. H.; Elmore, S. W. ABT-199, a potent and selective BCL-2 inhibitor, achieves antitumor activity while sparing platelets. Nat. Med. 2013, 19, 202-208.
44. Gadek, T. R.; Burdick, D. J.; McDowell, R. S.; Stanley, M. S.; Marsters, J. C., Jr.; Paris, K. J.; Oare, D. A.; Reynolds, M. E.; Ladner, C.; Zioncheck, K. A.; Lee, W. P.; Gribling, P.; Dennis, M. S.; Skelton, N. J.; Tumas, D. B.; Clark, K. R.; Keating, S. M.; Beresini, M. H.; Tilley, J. W.; Presta, L. G.; Bodary, S. C. Generation of an LFA-1 antagonist by the transfer of the ICAM-1 immunoregulatory epitope to a small molecule. Science 2002, 295, 1086-1089.
45. Tan, Q.; Zhu, Y.; Li, J.; Chen, Z.; Han, G. W.; Kufareva, I.; Li, T.; Ma, L.; Fenalti, G.; Li, J.; Zhang, W.; Xie, X.; Yang, H.; Jiang, H.; Cherezov, V.; Liu, H.; Stevens, R. C.; Zhao, Q.; Wu, B. Structure of the CCR5 chemokine receptor-HIV entry inhibitor maraviroc complex. Science 2013, 341, 1387-1390.
46. Neklesa, T. K.; Winkler, J. D.; Crews, C. M. Targeted protein degradation by PROTACs. Pharmacol. Ther. 2017, 174, 138-144.
47. Giannoukakis, N.; Phillips, B.; Trucco, M. Toward a cure for type 1 diabetes mellitus: diabetes-suppressive dendritic cells and beyond. Pediatr. Diabetes 2008, 9, 4-13.
48. Cochrane, G. M.; Horne, R.; Chanez, P. Compliance in asthma. Respir. Med. 1999, 93, 763-769.
49. Moia, M.; Mantovani, L. G.; Carpenedo, M.; Scalone, L.; Monzini, M. S.; Cesana, G.; Mannucci, P. M. Patient preferences and willingness to pay for different options of anticoagulant therapy. Intern. Emerg. Med. 2013, 8, 237-243.
50. Margolles-Clark, E.; Jacques-Silva, M. C.; Ganesan, L.; Umland, O.; Kenyon, N. S.; Ricordi, C.; Berggren, P.-O.; Buchwald, P. Suramin inhibits the CD40-CD154 costimulatory interaction: a possible mechanism for immunosuppressive effects. Biochem. Pharmacol. 2009, 77, 1236-1245.
51. Ganesan, L.; Margolles-Clark, E.; Song, Y.; Buchwald, P. The food colorant erythrosine is a promiscuous protein-protein interaction inhibitor. Biochem. Pharmacol. 2011, 81, 810-818.
52. Fletcher, S.; Hamilton, A. D. Targeting protein-protein interactions by rational design: mimicry of protein surfaces. J. R. Soc. Interface 2006, 3, 215-233.
53. Che, Y.; Brooks, B. R.; Marshall, G. R. Development of small molecules designed to modulate protein-protein interactions. J. Comput. Aided Mol. Des. 2006, 20, 109-130.
54. Hershberger, S. J.; Lee, S. G.; Chmielewski, J. Scaffolds for blocking protein-protein interactions. Curr. Top. Med. Chem. 2007, 7, 928-942.
55. Reynès, C.; Host, H.; Camproux, A. C.; Laconde, G.; Leroux, F.; Mazars, A.; Deprez, B.; Fahraeus, R.; Villoutreix, B. O.; Sperandio, O. Designing focused chemical libraries enriched in protein-protein interaction inhibitors using machine-learning methods. PLoS Comput. Biol. 2010, 6, e1000695.
56. Neugebauer, A.; Hartmann, R. W.; Klein, C. D. Prediction of protein-protein interaction inhibitors by chemoinformatics and machine learning methods. J. Med. Chem. 2007, 50, 4665-4668.
57. Sperandio, O.; Reynès, C. H.; Camproux, A. C.; Villoutreix, B. O. Rationalizing the chemical space of protein-protein interaction inhibitors. Drug Discov. Today 2010, 15, 220-229.
58. Levine, W. G. Metabolism of azo dyes: implication for detoxication and activation. Drug Metab. Rev. 1991, 23, 253-309.
59. Feng, J.; Cerniglia, C. E.; Chen, H. Toxicological significance of azo dye metabolism by human intestinal microbiota. Front. Biosci. 2012, 4, 568-586.
60. Tan, C. W.; Chia, W. N.; Qin, X.; Liu, P.; Chen, M. I.; Tiu, C.; Hu, Z.; Chen, V. C.; Young, B. E.; Sia, W. R.; Tan, Y. J.; Foo, R.; Yi, Y.; Lye, D. C.; Anderson, D. E.; Wang, L. F. A SARS-CoV-2 surrogate virus neutralization test based on antibody-mediated blockage of ACE2-spike protein-protein interaction. Nat. Biotechnol. 2020, 38, 1073-1078.
61. Colson, P.; Rolain, J. M.; Raoult, D. Chloroquine for the 2019 novel coronavirus SARS-CoV-2. Int. J. Antimicrob. Agents 2020, 55, 105923.
62. Gordon, D. E.; Jang, G. M.; Bouhaddou, M.; Xu, J.; Obernier, K.; White, K. M.; OMeara, M. J.; Rezelj, V. V.; Guo, J. Z.; Swaney, D. L.; Tummino, T. A.; Huettenhain, R.; Kaake, R. M.; Richards, A. L.; Tutuncuoglu, B.; Foussard, H.; Batra, J.; Haas, K.; Modak, M.; Kim, M.; Haas, P.; Polacco, B. J.; Braberg, H.; Fabius, J. M.; Eckhardt, M.; Soucheray, M.; Bennett, M. J.; Cakir, M.; McGregor, M. J.; Li, Q.; Meyer, B.; Roesch, F.; Vallet, T.; Mac Kain, A.; Miorin, L.; Moreno, E.; Naing, Z. Z. C.; Zhou, Y.; Peng, S.; Shi, Y.; Zhang, Z.; Shen, W.; Kirby, I. T.; Melnyk, J. E.; Chorba, J. S.; Lou, K.; Dai, S. A.; Barrio-Hernandez, I.; Memon, D.; Hernandez-Armenta, C.; Lyu, J.; Mathy, C. J. P.; Perica, T.; Pilla, K. B.; Ganesan, S. J.; Saltzberg, D. J.; Rakesh, R.; Liu, X.; Rosenthal, S. B.; Calviello, L.; Venkataramanan, S.; Liboy-Lugo, J.; Lin, Y.; Huang, X. P.; Liu, Y.; Wankowicz, S. A.; Bohn, M.; Safari, M.; Ugur, F. S.; Koh, C.; Savar, N. S.; Tran, Q. D.; Shengjuler, D.; Fletcher, S. J.; ONeal, M. C.; Cai, Y.; Chang, J. C. J.; Broadhurst, D. J.; Klippsten, S.; Sharp, P. P.; Wenzell, N. A.; Kuzuoglu, D.; Wang, H. Y.; Trenker, R.; Young, J. M.; Cavero, D. A.; Hiatt, J.; Roth, T. L.; Rathore, U.; Subramanian, A.; Noack, J.; Hubert, M.; Stroud, R. M.; Frankel, A. D.; Rosenberg, O. S.; Verba, K. A.; Agard, D. A.; Ott, M.; Emerman, M.; Jura, N.; von Zastrow, M.; Verdin, E.; Ashworth, A.; Schwartz, O.; dEnfert, C.; Mukherjee, S.; Jacobson, M.; Malik, H. S.; Fujimori, D. G.; Ideker, T.; Craik, C. S.; Floor, S. N.; Fraser, J. S.; Gross, J. D.; Sali, A.; Roth, B. L.; Ruggero, D.; Taunton, J.; Kortemme, T.; Beltrao, P.; Vignuzzi, M.; Garcia-Sastre, A.; Shokat, K. M.; Shoichet, B. K.; Krogan, N. J. A SARS-CoV-2 protein interaction map reveals targets for drug repurposing. Nature 2020, 583, 459-468.
63. McKee, D. L.; Sternberg, A.; Stange, U.; Laufer, S.; Naujokat, C. Candidate drugs against SARS-CoV-2 and COVID-19. Pharmacol. Res. 2020, 157, 104859.
64. Salgado-Benvindo, C.; Thaler, M.; Tas, A.; Ogando, N. S.; Bredenbeek, P. J.; Ninaber, D. K.; Wang, Y.; Hiemstra, P. S.; Snijder, E. J.; van Hemert, M. J. Suramin inhibits SARS-CoV-2 infection in cell culture by interfering with early steps of the replication cycle. Antimicrob. Agents Chemother. 2020, 64, e00900-20.
65. Clifton, J., 2nd; Leikin, J. B. Methylene blue. Am. J. Ther. 2003, 10, 289-291.
66. Bistas, E.; Sanghavi, D. Methylene blue In StatPearls: Treasure Island; FL, USA, 2020.
67. Dicko, A.; Roh, M. E.; Diawara, H.; Mahamar, A.; Soumare, H. M.; Lanke, K.; Bradley, J.; Sanogo, K.; Kone, D. T.; Diarra, K.; Keita, S.; Issiaka, D.; Traore, S. F.; McCulloch, C.; Stone, W. J. R.; Hwang, J.; Muller, O.; Brown, J. M.; Srinivasan, V.; Drakeley, C.; Gosling, R.; Chen, I.; Bousema, T. Efficacy and safety of primaquine and methylene blue for prevention of Plasmodium falciparum transmission in Mali: a phase 2, single-blind, randomised controlled trial. Lancet Infect. Dis. 2018, 18, 627-639.
68. Lozano, M.; Cid, J.; Muller, T. H. Plasma treated with methylene blue and light: clinical efficacy and safety profile. Transfus. Med. Rev. 2013, 27, 235-240.
69. Cagno, V.; Medaglia, C.; Cerny, A.; Cerny, T.; Tapparel, C.; Cerny, E. Methylene blue has a potent antiviral activity against SARS-CoV-2 in the absence of UV-activation in vitro. BioRxiv 2020, 2020.08.14.251090.
70. Walter-Sack, I.; Rengelshausen, J.; Oberwittler, H.; Burhenne, J.; Mueller, O.; Meissner, P.; Mikus, G. High absolute bioavailability of methylene blue given as an aqueous oral formulation. Eur. J. Clin. Pharmacol. 2009, 65, 179-189.
71. Baddeley, T. C.; McCaffrey, J.; Storey, J. M.; Cheung, J. K.; Melis, V.; Horsley, D.; Harrington, C. R.; Wischik, C. M. Complex disposition of methylthioninium redox forms determines efficacy in tau aggregation inhibitor therapy for Alzheimer® disease. J. Pharmacol. Exp. Ther. 2015, 352, 110-118.
72. Golwalkar, D. Treatment for COVID-19 using methylene blue. Medium 2020, https://medium.com/@dr.deepak.golwalkar/treatment-for-covid-19-using-methylene-blue-d23fc5a31a4d.
73. Tobin, M. J.; Laghi, F.; Jubran, A. Why COVID-19 silent hypoxemia is baffling to physicians. Am. J. Respir. Crit. Care Med. 2020, 202, 356-360.
74. Schenk, P.; Madl, C.; Rezaie-Majd, S.; Lehr, S.; Muller, C. Methylene blue improves the hepatopulmonary syndrome. Ann. Intern. Med. 2000, 133, 701-706.
75. Henry, M.; Summa, M.; Patrick, L.; Schwartz, L. A cohort of cancer patients with no reported cases of SARS-CoV-2 infection: the possible preventive role of methylene blue. Substantia 2020, 4, 888.
76. Alamdari, D. H.; Moghaddam, A. B.; Amini, S.; Keramati, M. R.; Zarmehri, A. M.; Alamdari, A. H.; Damsaz, M.; Banpour, H.; Yarahmadi, A.; Koliakos, G. Application of methylene blue—vitamin C—N-acetyl cysteine for treatment of critically ill COVID-19 patients, report of a phase-I clinical trial. Eur. J. Pharmacol. 2020, 173494.
77. Schneider, C. A.; Rasband, W. S.; Eliceiri, K. W. NIH Image to ImageJ: 25 years of image analysis. Nat. Methods 2012, 9, 671-675.
78. Venkatraj, M.; Messagie, J.; Joossens, J.; Lambeir, A. M.; Haemers, A.; Van der Veken, P.; Augustyns, K. Synthesis and evaluation of non-basic inhibitors of urokinase-type plasminogen activator (uPA). Bioorg. Med. Chem. 2012, 20, 1557-1568.
79. Kassack, M. U.; Braun, K.; Ganso, M.; Ullmann, H.; Nickel, P.; Boing, B.; Muller, G.; Lambrecht, G. Structure-activity relationships of analogues of NF449 confirm NF449 as the most potent and selective known P2X1 receptor antagonist. Eur. J. Med. Chem. 2004, 39, 345-357.
80. Condor Capcha, J. M.; Lambert, G.; Dykxhoorn, D. M.; Salerno, A. G.; Hare, J. M.; Whitt, M. A.; Pahwa, S.; Jayaweera, D. T.; Shehadeh, L. A. Generation of SARS-CoV-2 spike pseudotyped virus for viral entry and neutralization assays: a 1-week protocol. Front. Cardiovasc. Med. 2020, 7, 618651.
81. Bojadzic, D.; Alcazar, O.; Chen, J.; Chuang, S. T.; Condor Capcha, J. M.; Shehadeh, L. A.; Buchwald, P. Small-molecule inhibitors of the coronavirus spike—ACE2 protein-protein interaction as blockers of viral attachment and entry for SARS-CoV-2. ACS Infect. Dis. 2021, 7, 1519-1534.

1. A method of preventing or treating a viral infection in a subject in need thereof, the method comprising administering to the subject a therapeutically effective amount of a compound of Formula (I): 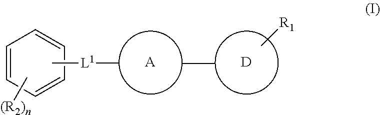
wherein:
Ring A is 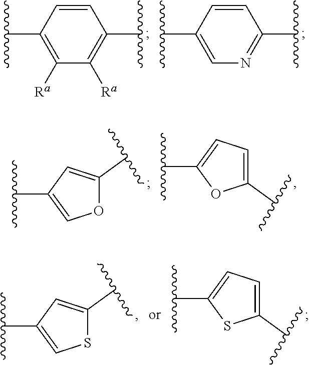
Ring D is 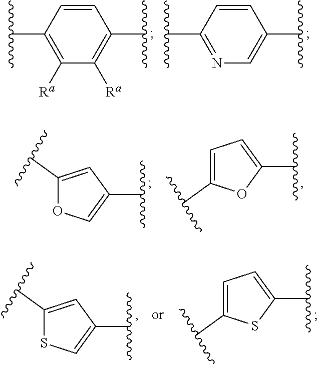
R₁ is H, halo, CF₃, SO₃H, CO₂R^{b}, NO₂, NH₂, or 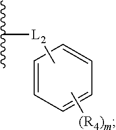
each of L₁ and L₂ independently is 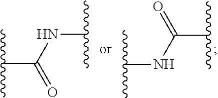 [SMILES: *NC(*)=O ; *NC(*)=O]
n is 0, 1, 2, 3, or 4;
m is 0, 1, 2, 3, or 4;
each R₂ independently is halo, CF₃, SO₃H, CO₂R^{b}, NO₂, or NH₂; and when two R₂ are adjacent, they can together form —(N═N—NH)— or, with the carbon atoms to which they are attached, form a C₆ aryl optionally substituted with 1-4 R₃;
each R₃ independently is halo, OH, CF₃, SO₃H, CO₂R^{b}, NO₂, or NH₂; and when two R₃ are adjacent, they can together form —(N═N—NH)—;
each R₄ independently is halo, CF₃, SO₃H, CO₂R^{b}, NO₂, or NH₂; and when two R₄ are adjacent, they can together form —(N═N—NH)— or, with the carbon atoms to which they are attached, form a C₆ aryl optionally substituted with 1-4 R₃;
each R^{a} independently is H, C₁₋₅ alkyl, or C₁₋₅ alkoxy; and
each R^{b} independently is H or C₁₋₅ alkyl;
or a pharmaceutically acceptable salt thereof.

2. The method of claim 1, wherein the viral infection is caused by a coronavirus.

3. The method of claim 2, wherein the coronavirus is selected from the group consisting of SARS-CoV, SARS-CoV-2, HCoV-NL63, MERS-CoV, HCoV-229E, HCoV-OC43, HCoV-HKU1, and a combination thereof.

4. The method of claim 1, wherein the compound of Formula (I) treats or prevents the viral infection by inhibiting an interaction between a coronavirus spike protein and a receptor thereof, thereby decreasing viral attachment and entry into a host cell.

5. The method of claim 4, wherein the receptor is angiotensin converting enzyme 2 (ACE2), dipeptidyl peptidase 4 (DPP4) or CD13.

6.-15. (canceled)

16. The method of claim 1, wherein 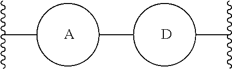
is selected from the group consisting of

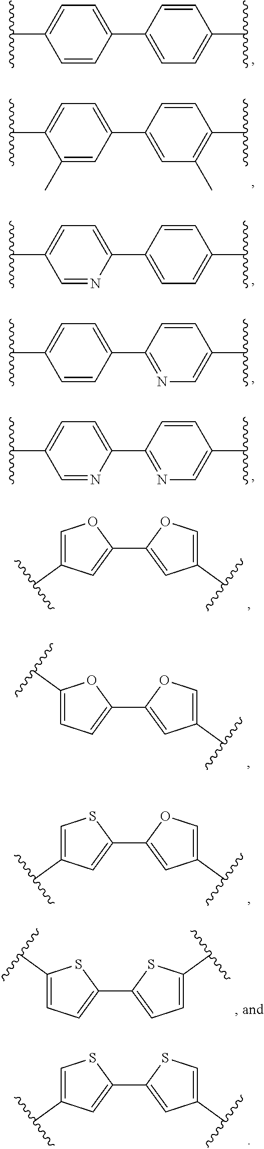

17.-22. (canceled)

23. The method of claim 1, wherein R₁ is CO₂R^{b} and R^{b} is H.

24.-27. (canceled)

28. The method of claim 1, wherein R₁ is

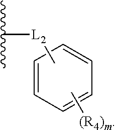

29.-31. (canceled)

32. The method of claim 28, wherein m is 1, 2, 3, or 4.

33.-41. (canceled)

42. The method of claim 32, wherein m is at least 2, and two R₄ are adjacent and together with the carbon atoms to which they are attached form a C₆ aryl optionally substituted with 1-4 R₃.

43. The method of claim 1, wherein R₁ is selected from the group consisting of

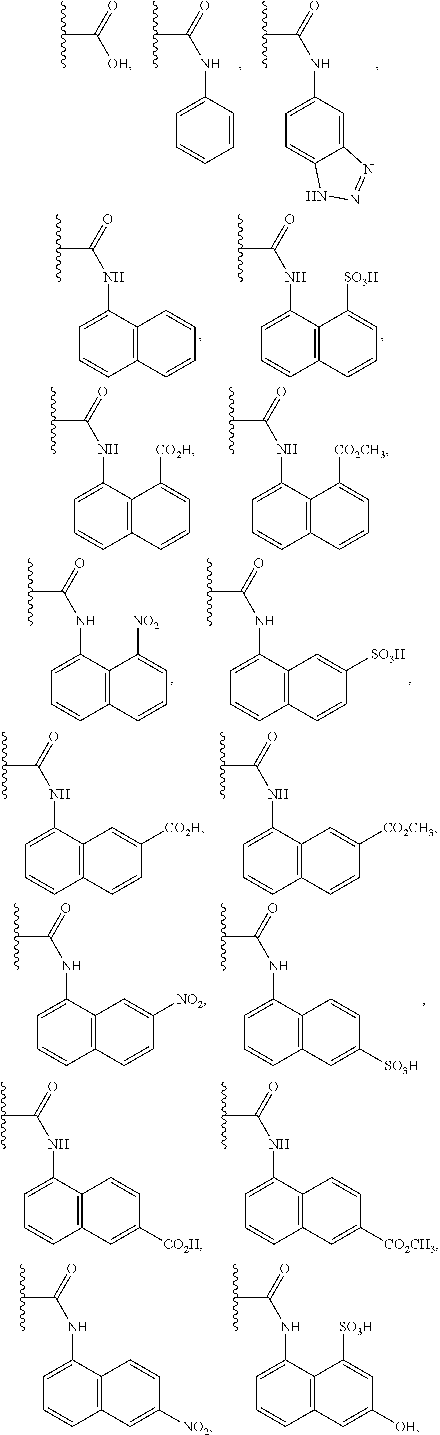

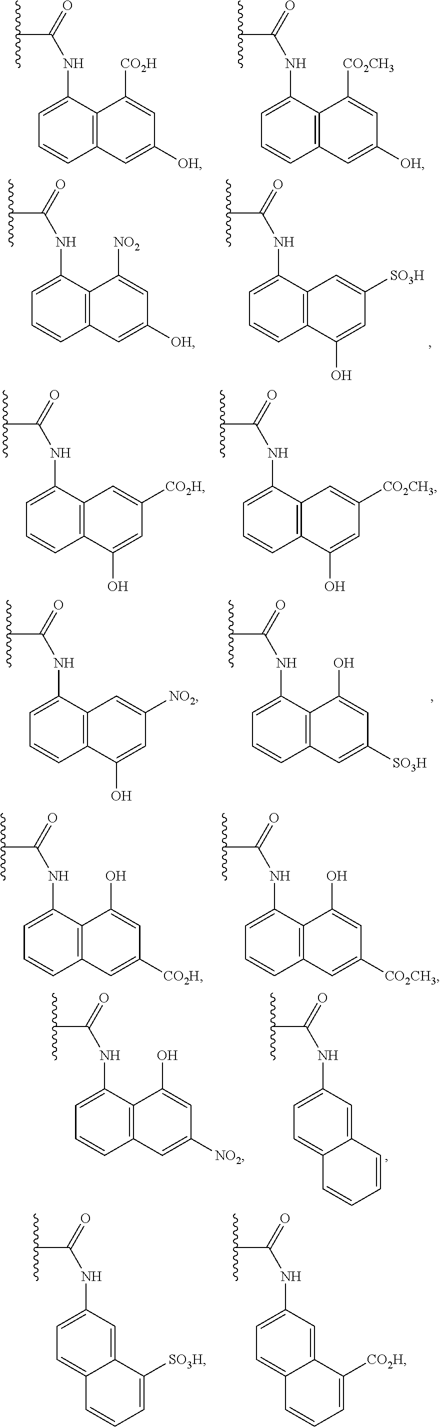

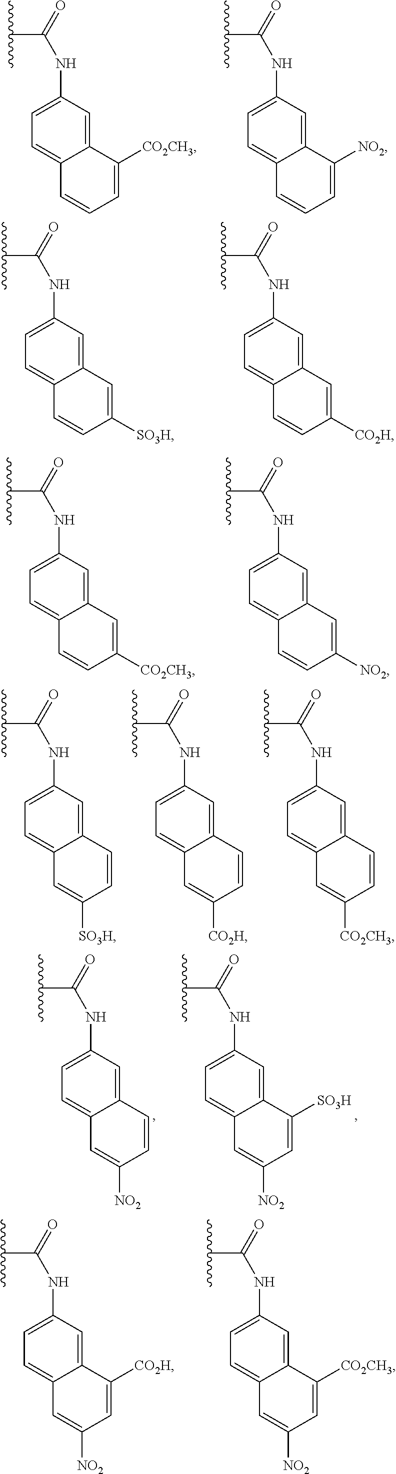

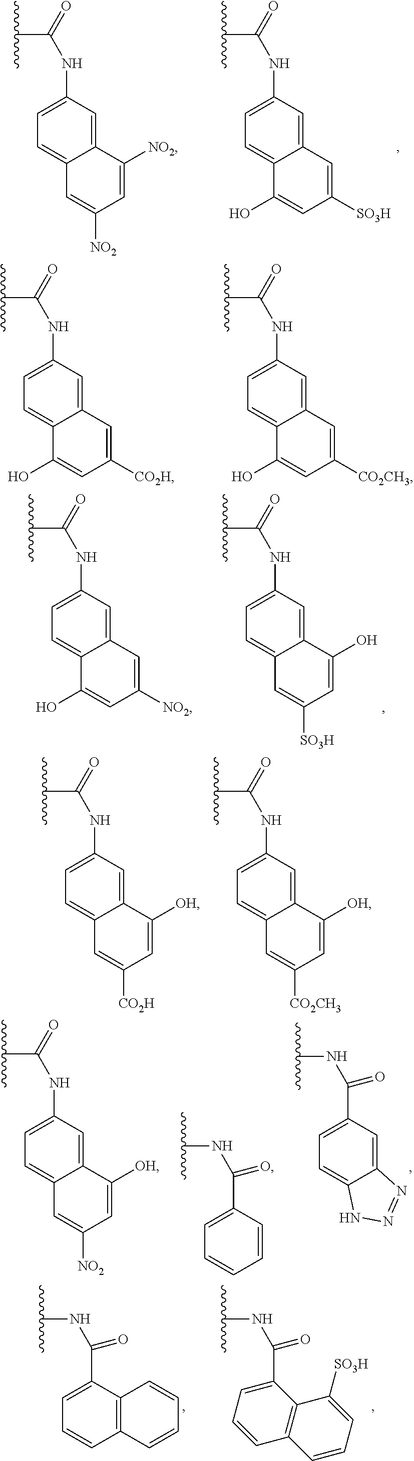

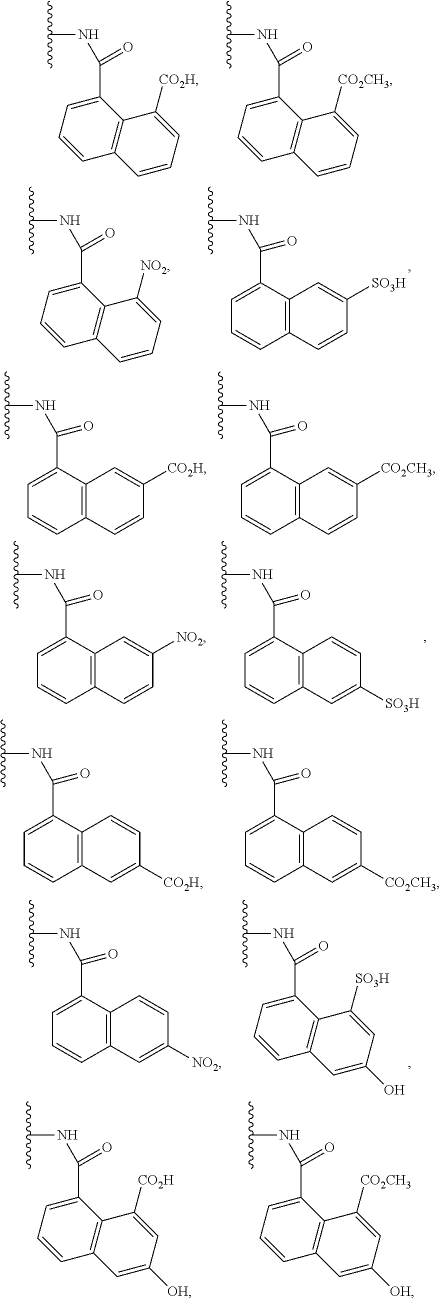

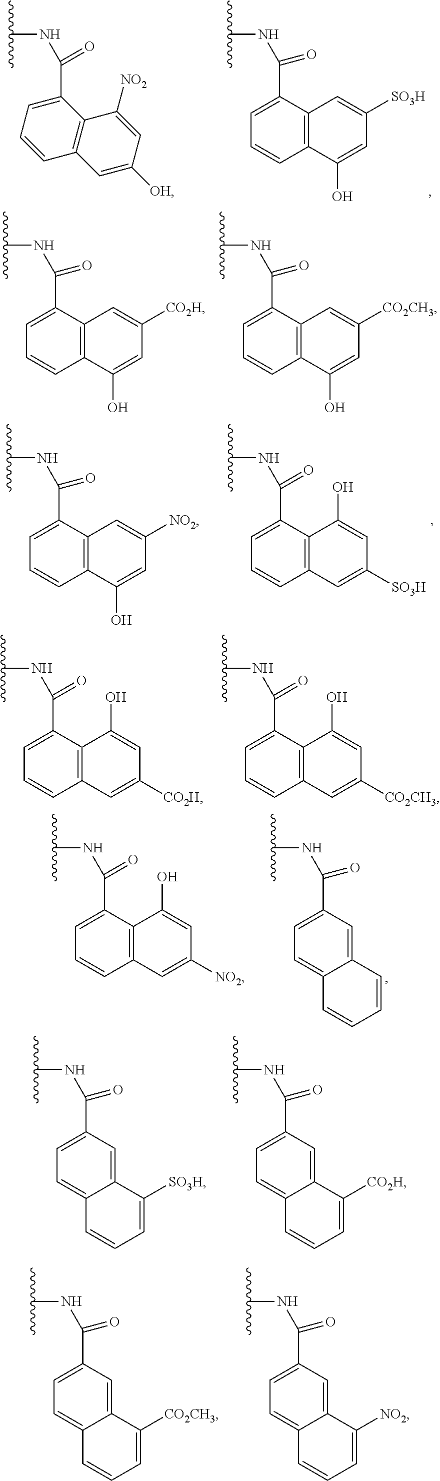

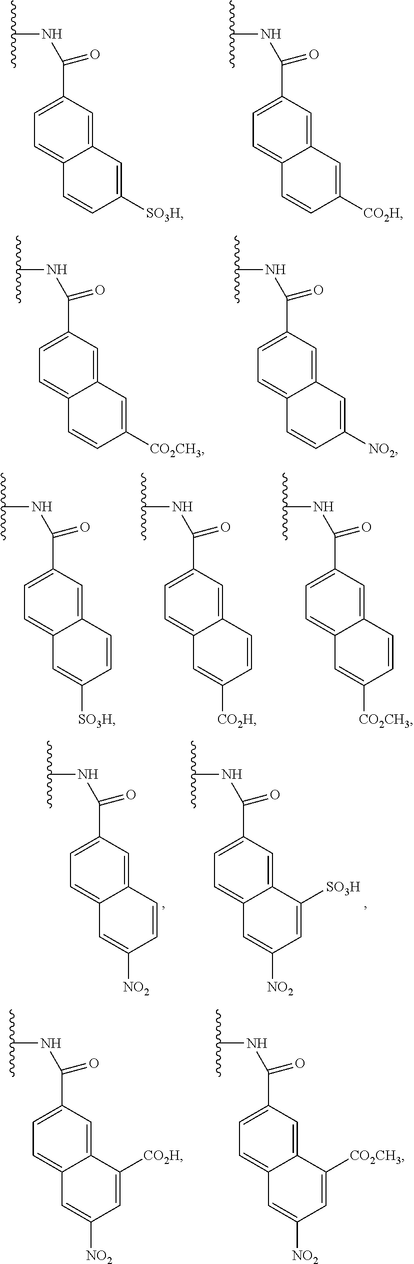

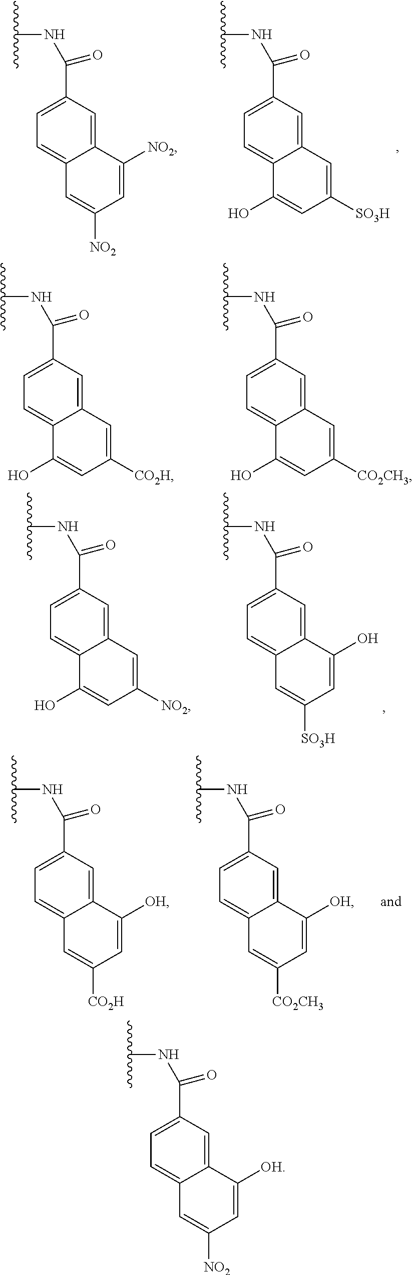

44.-46. (canceled)

47. The method of claim 1, wherein n is 1, 2, 3, or 4.

48.-54. (canceled)

55. The method of claim 47, wherein at least one R₂ is NO₂.

56. (canceled)

57. The method of claim 1, wherein n is at least 2, and two R₂ are adjacent and taken together with the carbon atoms to which they are attached form a C₆ aryl optionally substituted with 1-4 R₃.

58. The method of claim 1, wherein 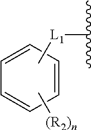
is selected from the group consisting of,

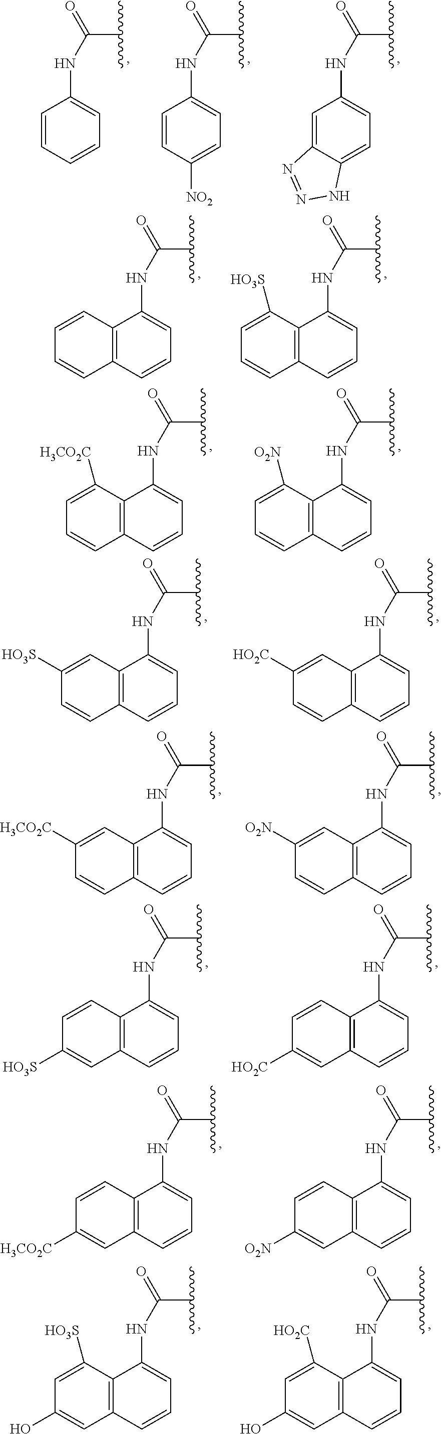

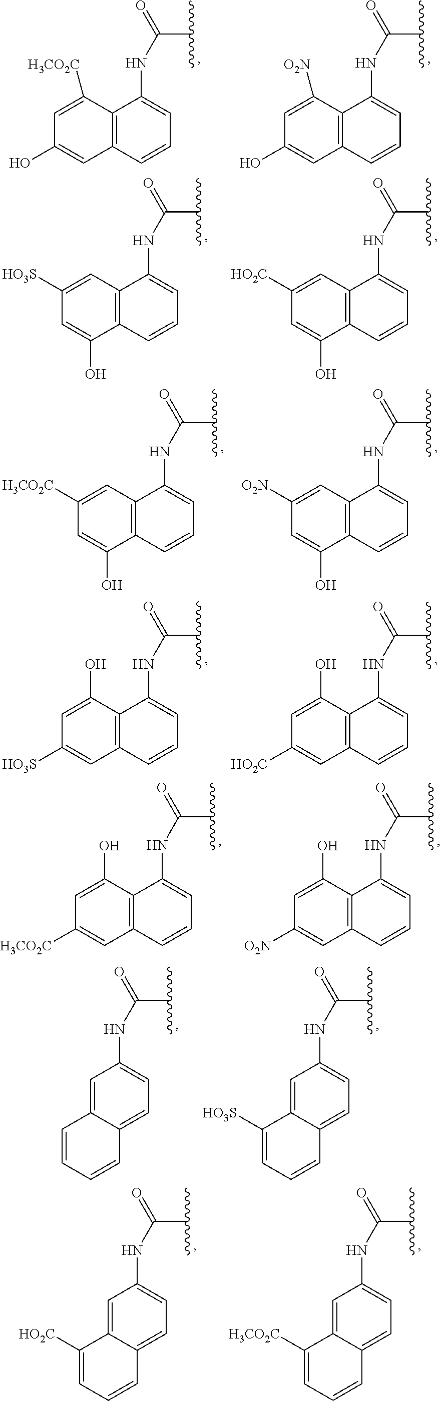

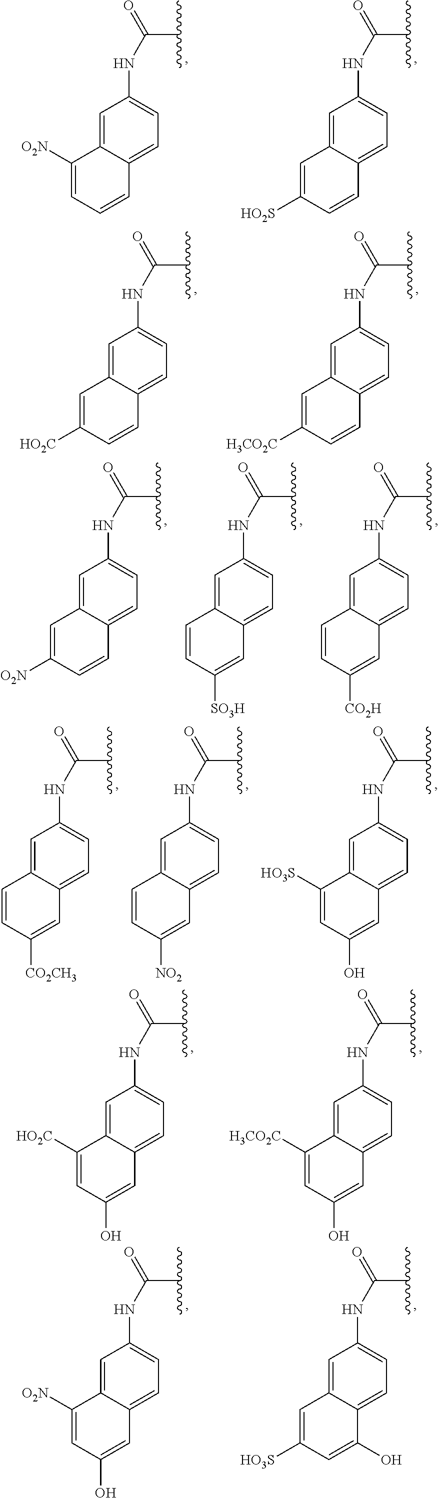

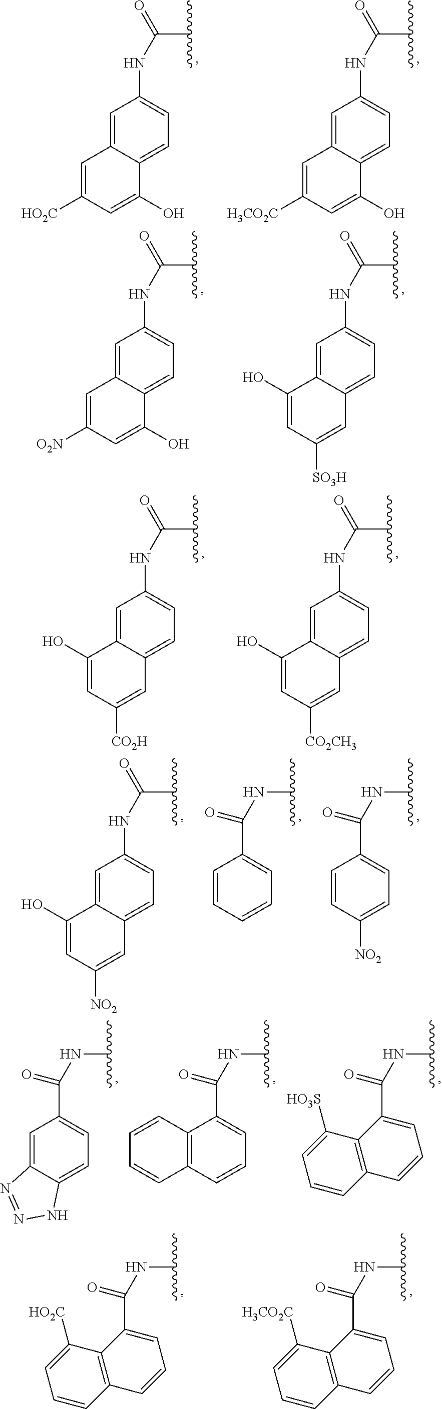

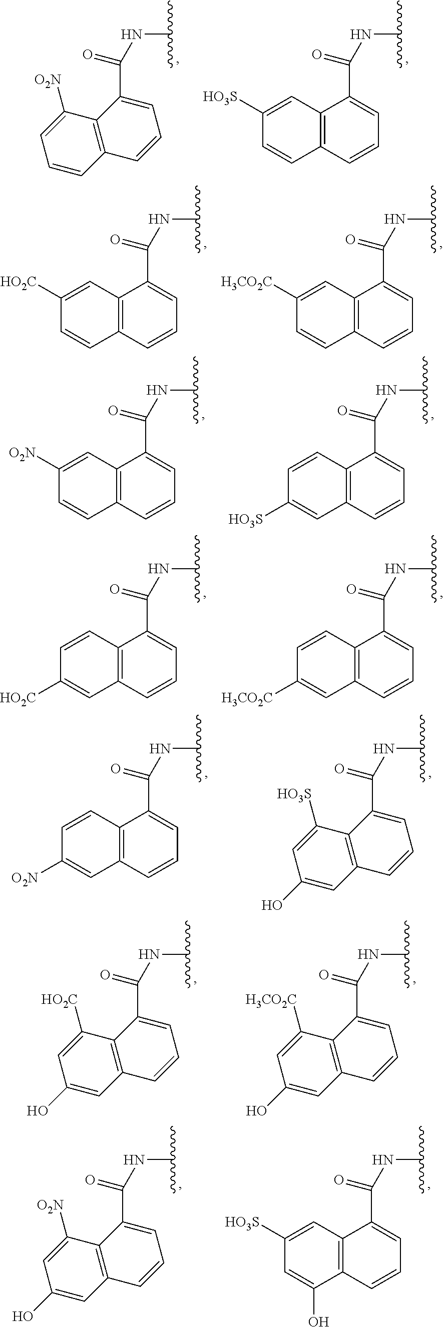

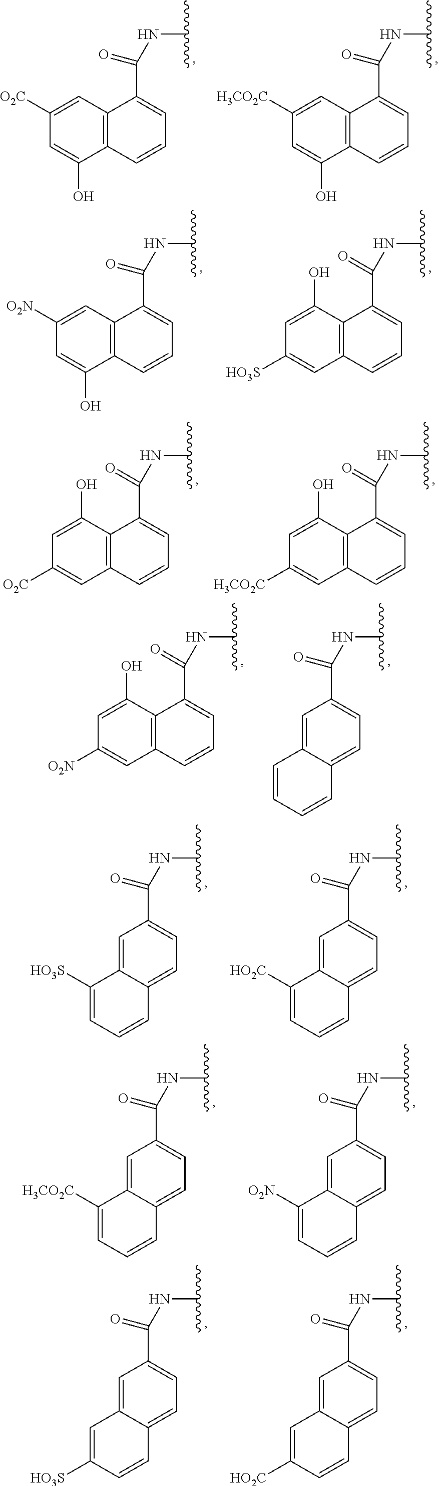

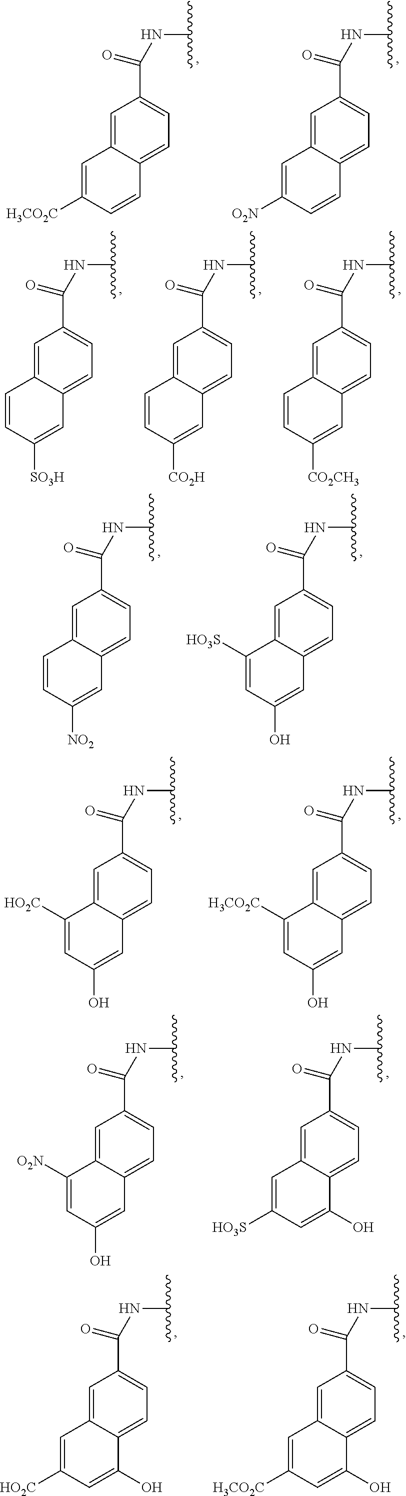

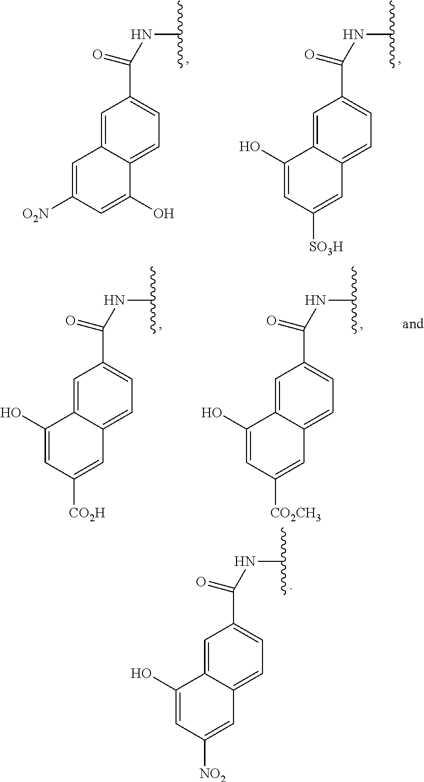 [SMILES: *NC(=O)c1ccc2c(O)cc([N+](=O)[O-])cc2c1 ; *NC(=O)c1ccc2cc(S(=O)(=O)O)cc(O)c2c1 ; *NC(=O)c1ccc2cc(C(=O)O)cc(O)c2c1 ; *NC(=O)c1ccc2cc(C(=O)OC)cc(O)c2c1 ; *NC(=O)c1ccc2cc([N+](=O)[O-])cc(O)c2c1]

59. The method of claim 1, wherein the compound or salt is selected from the group consisting of

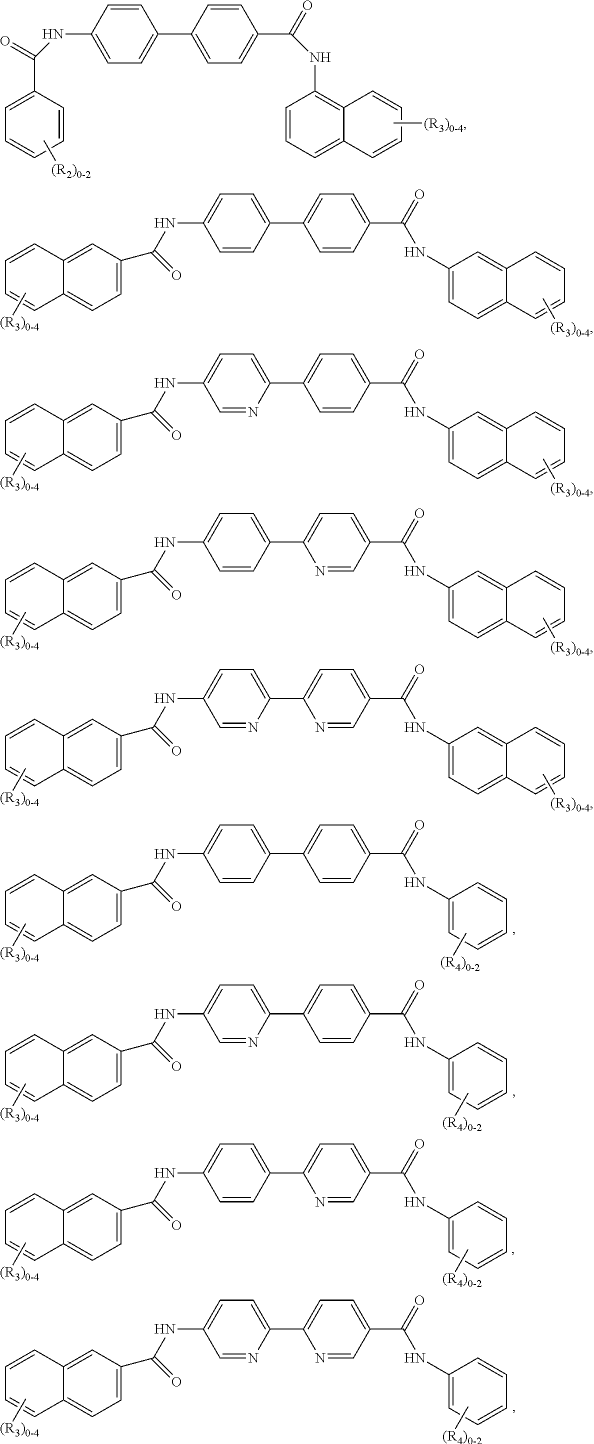

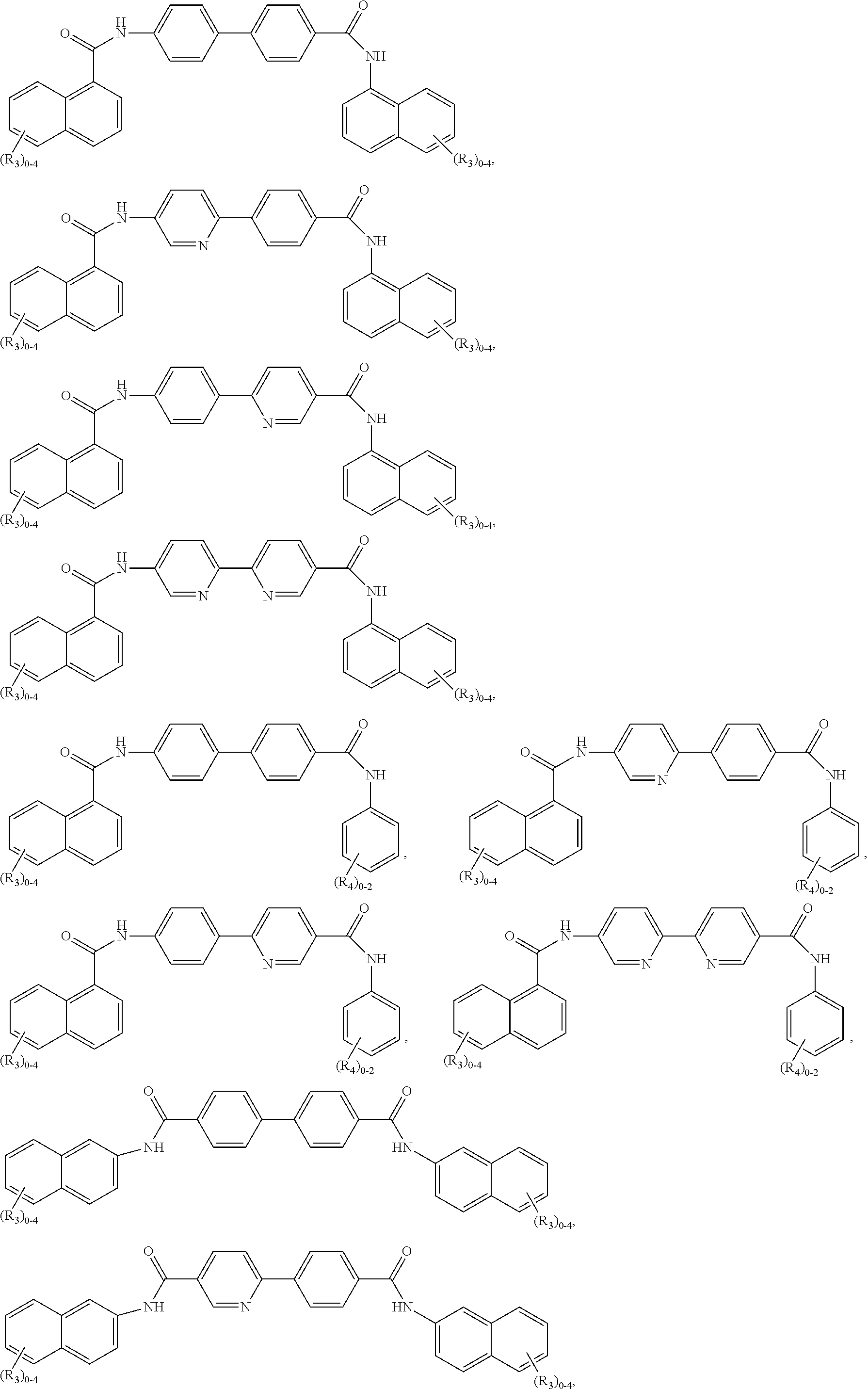

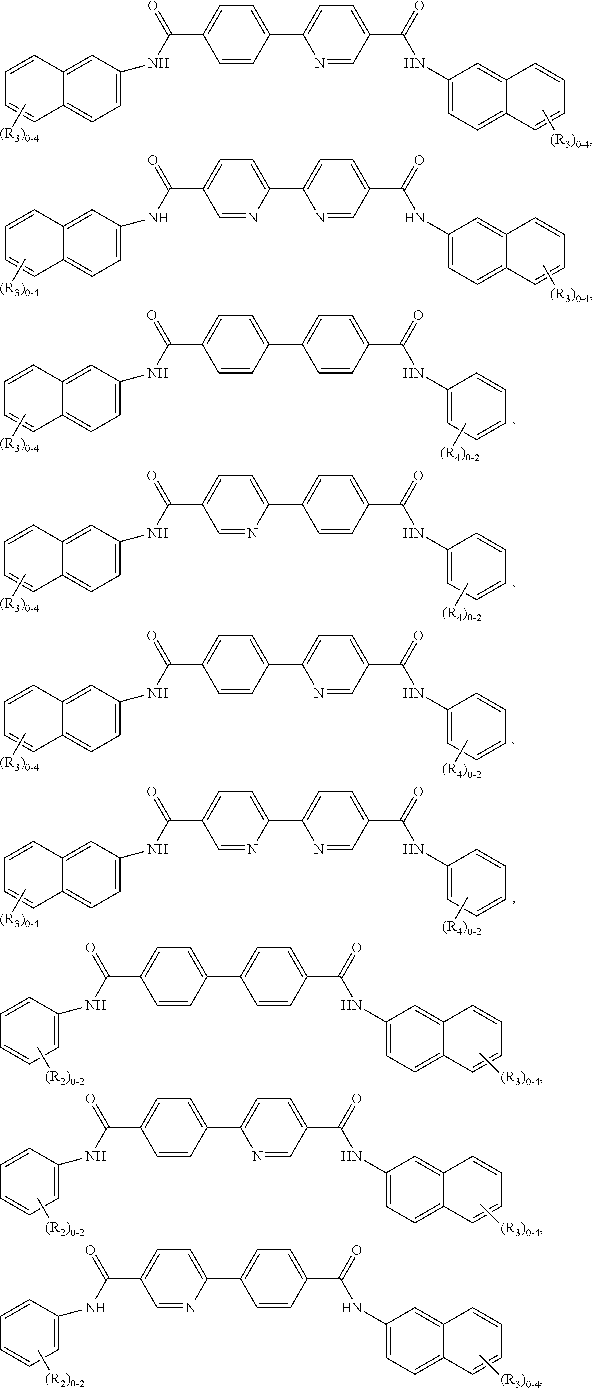

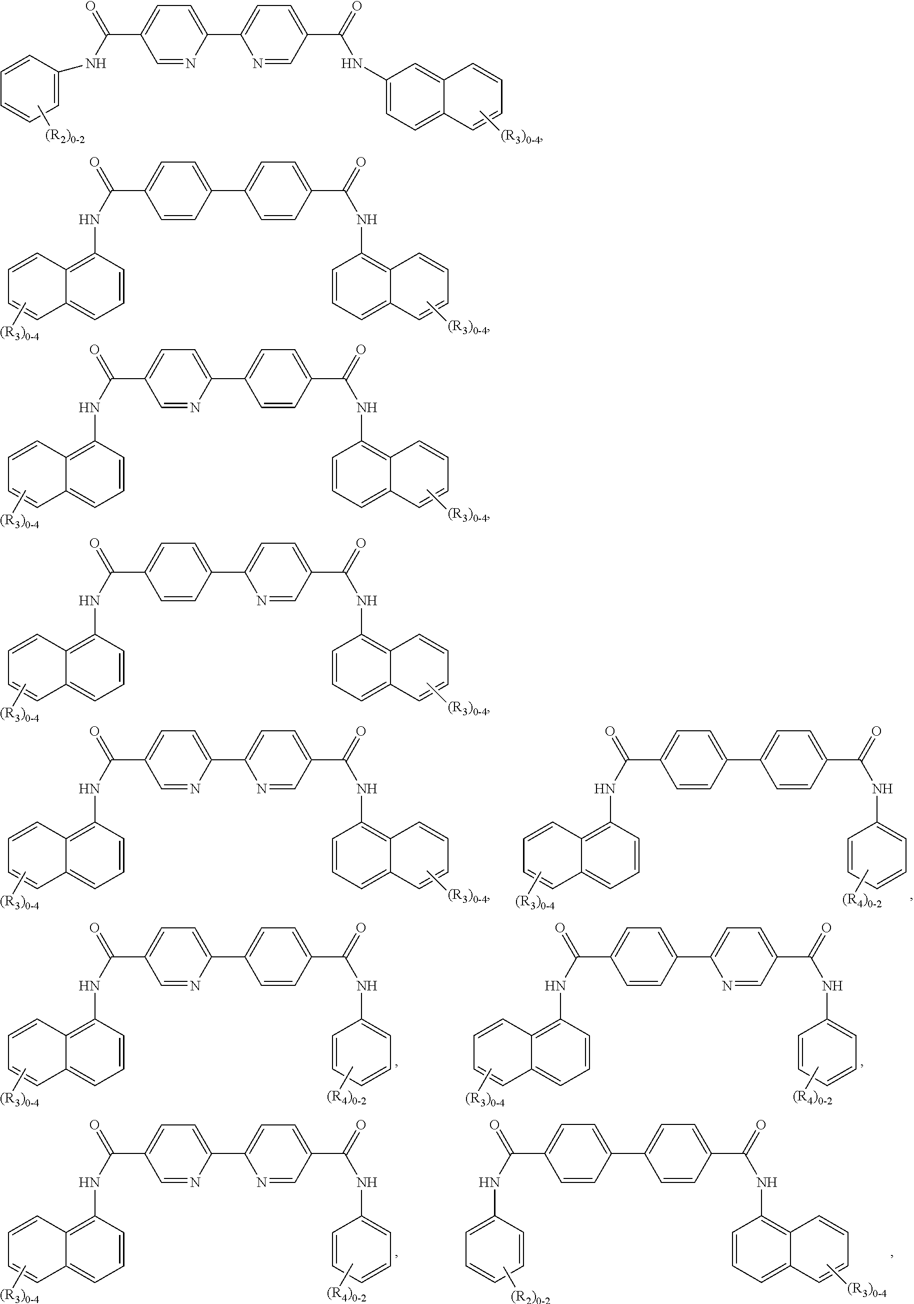

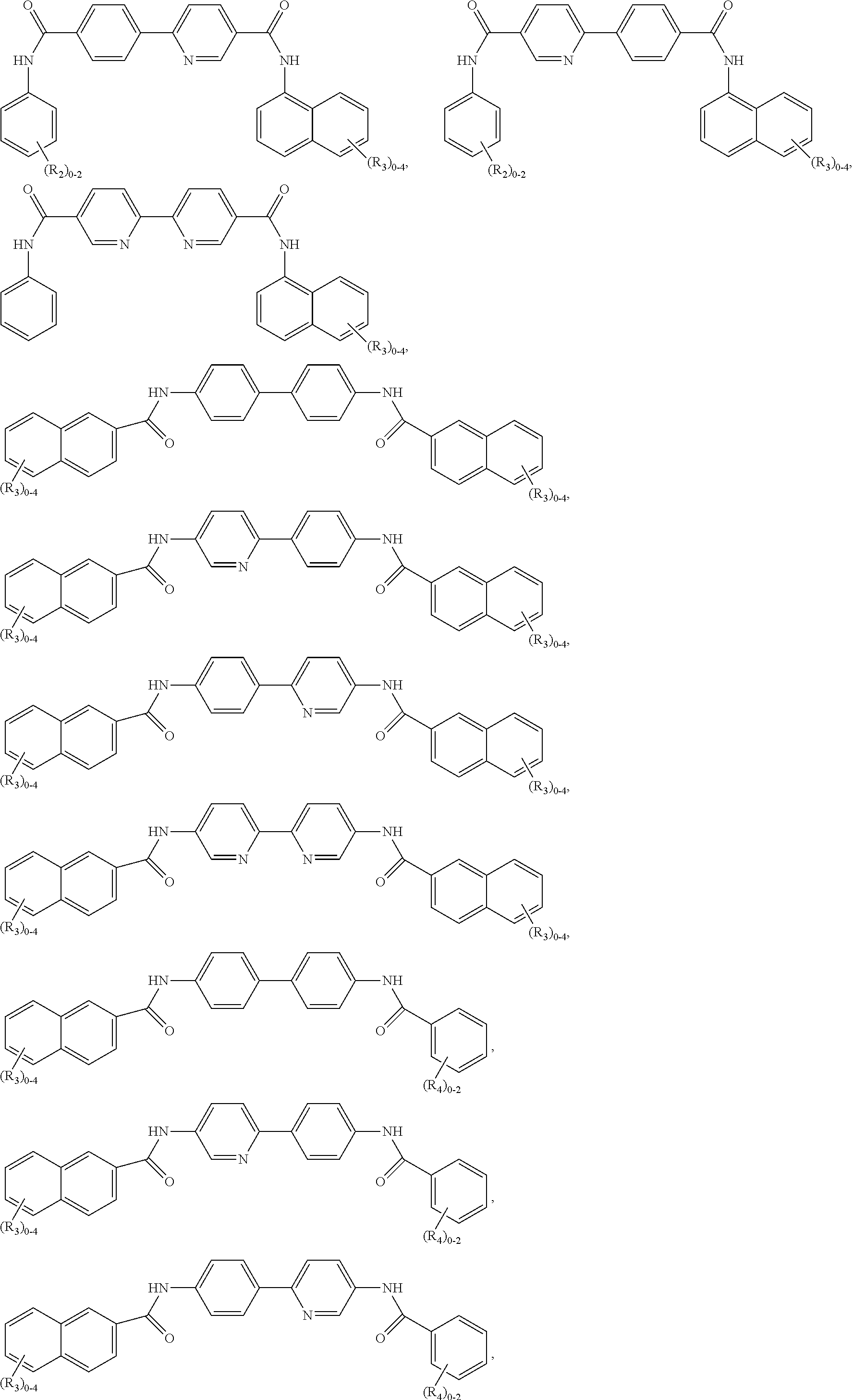

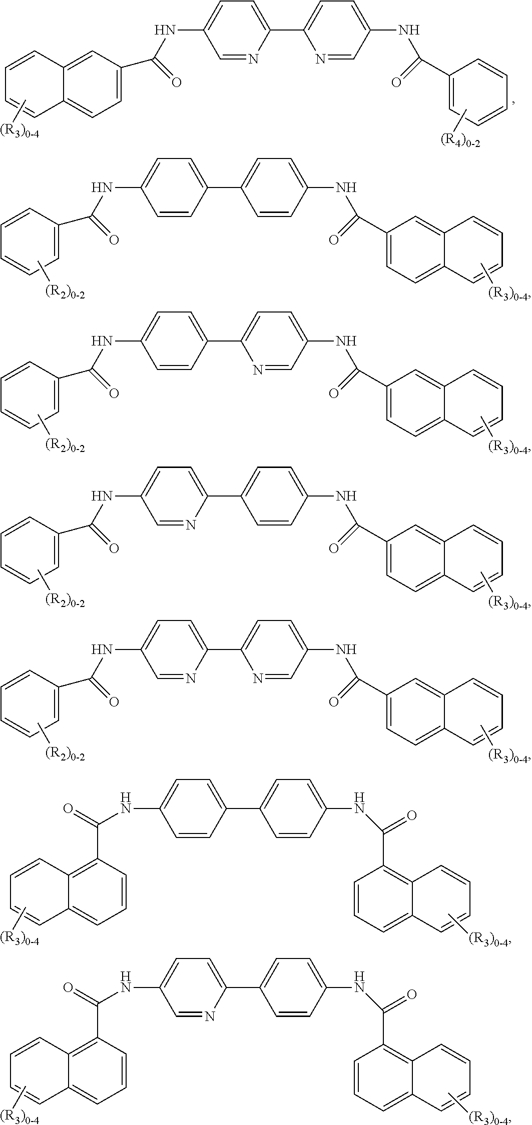

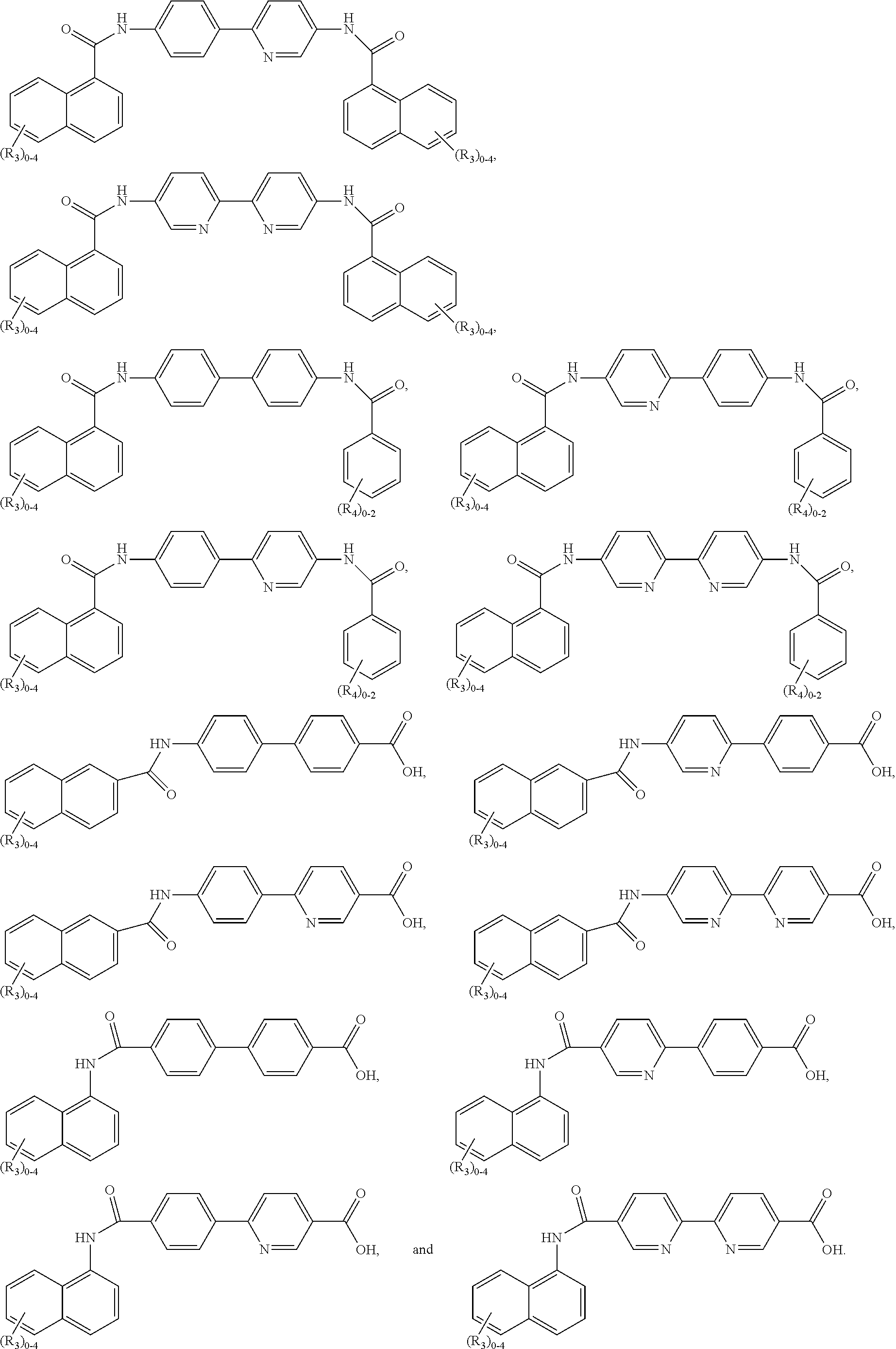

60. The method of claim 1, wherein the compound or salt is selected from the group consisting of

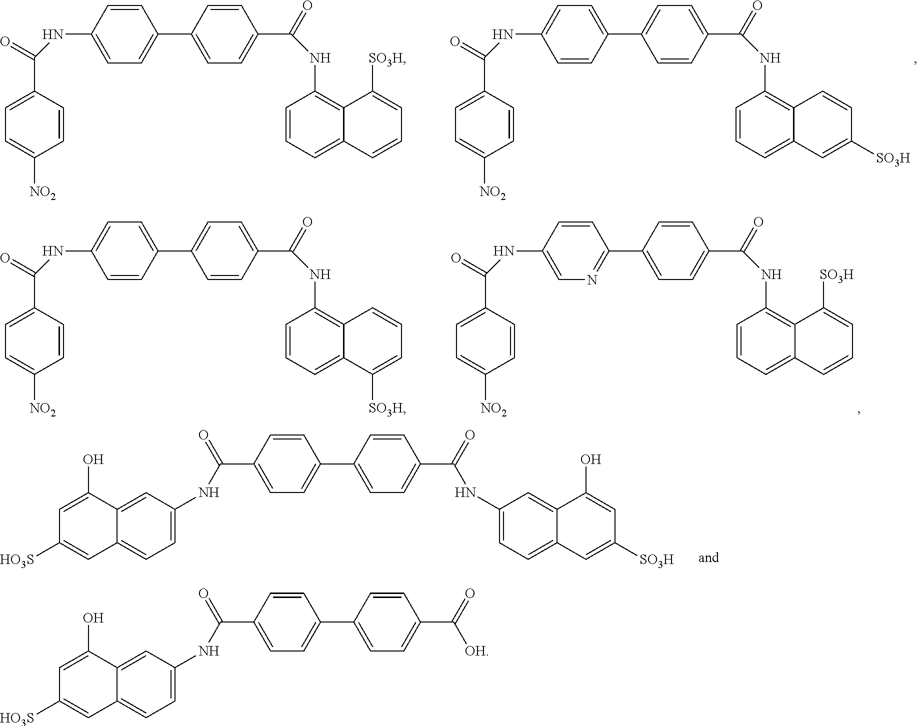

61. A compound of Formula (I): 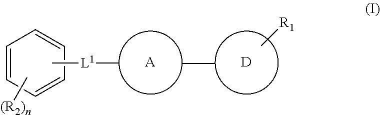
wherein:
Ring A is 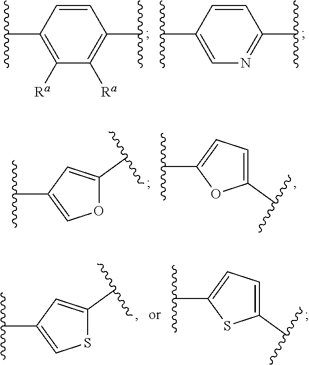
Ring D is 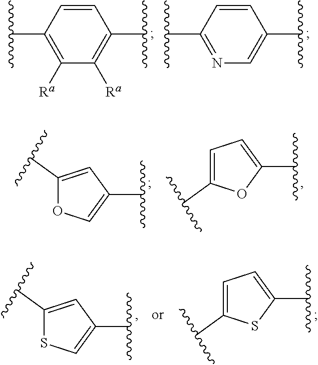
R₁ is H, halo, CF₃, SO₃H, CO₂R^{b}, NO₂, NH₂, or 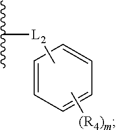
each of L₁ and L₂ independently is 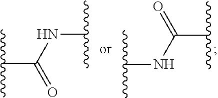 [SMILES: *NC(*)=O ; *NC(*)=O]
n is 0, 1, 2, 3, or 4;
m is 0, 1, 2, 3, or 4;
each R₂ independently is halo, CF₃, SO₃H, CO₂R^{b}, NO₂, or NH₂; and when two R₂ are adjacent, they can together form —(N═N—NH)— or, with the carbon atoms to which they are attached, form a C₆ aryl optionally substituted with 1-4 R₃;
each R₃ independently is halo, OH, CF₃, SO₃H, CO₂R^{b}, NO₂, or NH₂; and when two R₃ are adjacent, they can together form —(N═N—NH)—;
each R₄ independently is halo, CF₃, SO₃H, CO₂R^{b}, NO₂, or NH₂; and when two R₄ are adjacent, they can together form —(N═N—NH)— or, with the carbon atoms to which they are attached, form a C₆ aryl optionally substituted with 1-4 R₃;
each R^{a} independently is H, C₁₋₅ alkyl, or C₁₋₅ alkoxy; and,
each R^{b} independently is H or C₁₋₅ alkyl;
or a pharmaceutically acceptable salt thereof;
with the proviso that:
(a) if L₁-ring A-ring D-L₂ is 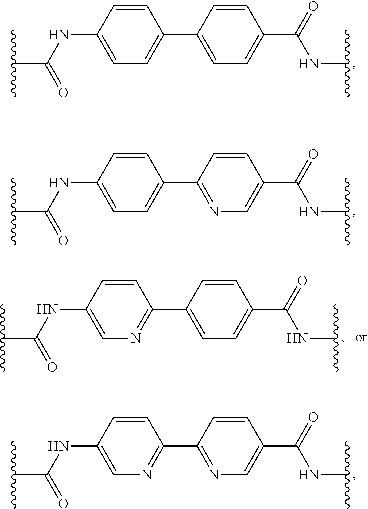 [SMILES: *NC(=O)c1ccc(-c2ccc(NC(*)=O)cc2)cc1 ; *NC(=O)c1ccc(-c2ccc(NC(*)=O)cc2)nc1 ; *NC(=O)c1ccc(-c2ccc(NC(*)=O)cn2)cc1 ; *NC(=O)c1ccc(-c2ccc(NC(*)=O)cn2)nc1]
then two adjacent R₂, with the carbon atoms to which they are attached, form a C₆ aryl, optionally substituted with 1-4 R₃; and
(b) if L₁-ring A-ring D-L₂ is 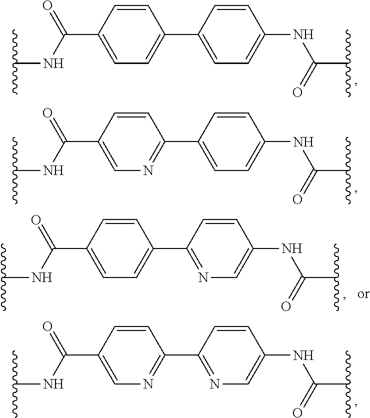
then two adjacent R₄, with the carbon atoms to which they are attached, form a C₆ aryl, optionally substituted with 1-4 R₃.

62.-116. (canceled)

117. A pharmaceutical composition comprising the compound or salt of claim 61 a pharmaceutically acceptable carrier.

118. Use of the compound or salt of claim 61 as a medicament in a mammal.

119.-123. (canceled)
